(19) United States
(12) Patent Application Publication
(10) Pub. No.: US 2026/0043072 A1
(43) Pub. Date: Feb. 12, 2026

(54) METHODS, COMPOSITIONS, AND SYSTEMS FOR BARCODING AND SPATIAL ANALYSIS

(71) Applicant: 10x Genomics, Inc., Pleasanton, CA (US)

(72) Inventor: Octavian Marian BLOJU, Stockholm (SE)

(21) Appl. No.: 19/294,781

(22) Filed: Aug. 8, 2025

Related U.S. Application Data
(60) Provisional application No. 63/681,700, filed on Aug. 9, 2024.

(51) Int. Cl. C12Q 1/6841; C12Q 1/6874

(52) U.S. Cl. CPC C12Q 1/6841; C12Q 1/6874

(57) ABSTRACT

The present disclosure relates in some aspects to methods, compositions, and kits of processing or analyzing a sample. A method for processing a sample may comprise providing probes with barcodes to generate a composite barcode, detecting the barcodes to determine a spatial location and hybridizing probes to analytes (e.g., an RNA molecule) and performing sequencing.

### CROSS-REFERENCE TO RELATED APPLICATIONS

This application claims benefit of and priority to U.S. Provisional Application No. 63/681,700, filed on Aug. 9, 2024, entitled “Methods, Compositions, and Systems for Barcoding and Spatial Analysis”, which is hereby incorporated by reference in its entirety.

### FIELD

The present disclosure relates in some aspects to methods, compositions, and systems for barcoding analytes and determining spatial locations of the analytes in biological samples.

### BACKGROUND

Profiling biological targets in a sample, such as genomic, transcriptomic, or proteomic profiling of cells, are essential for many purposes, such as understanding the molecular basis of cell identity and developing treatment for diseases. Whole genome amplification and sequencing technologies are beginning to find broader adoption. However, these technologies may not provide spatial information of analytes. Therefore, there is a need for new and improved methods for analyzing various analytes using sequencing technologies that also provides information regarding their spatial locations in a biological sample.

### SUMMARY

In some aspects, the present application provides new and improved methods, compositions, and systems for profiling biological targets (analytes) in a sample to determine a spatial location of the analytes (e.g., in a cell) in a biological sample. In some aspects, probes with barcodes are provided to generate a composite barcode and the barcodes are detected in situ to determine a spatial location before hybridizing probes to analytes (e.g., an RNA molecule) and performing barcoding and subsequent sequencing. While whole genome amplification and sequencing technologies provide genomic, transcriptomic, and/or proteomic profiling of cells, these techniques may not provide spatial information of analytes. Therefore, there is a need for new and improved methods for analyzing various analytes using sequencing technologies that also provides information regarding their spatial locations in a biological sample.

Provided herein is a method comprising (a) contacting a plurality of cells in the biological sample with a plurality of probes to generate a composite barcode in a cell of the plurality of cells, wherein the probes of the plurality of probes comprise a target binding sequence and a barcode sequence, wherein the target binding sequences of the probes in the plurality of probes are the same, and the probes of the plurality of probes comprise different barcode sequences; wherein the composite barcode in the cell comprises two or more different barcode sequences from separate probes of the plurality of probes; (b) detecting the two or more different barcode sequences of the composite barcode or complements thereof in the cell at a location in the biological sample; (c) dissociating cells or nuclei of the plurality of cells from the biological sample; (d) subjecting the dissociated cells or dissociated nuclei of the cells to a single cell barcoding reaction to append a common cell barcode or complement thereof to (i) a nucleic acid molecule comprising a barcode sequence of the two or more different barcode sequences of the composite barcode, and (ii) a sequence of or associated with an analyte from the cell, thereby generating a plurality of barcoded nucleic acid products; and (e) using the common cell barcode and the location of the detected composite barcode to locate the analyte in the biological sample.

In some instances, the dissociated cells or dissociated nuclei are partitioned with a plurality of nucleic acid barcode molecules in a partition, wherein each nucleic acid barcode molecule of the plurality of nucleic acid barcode molecules comprises a capture sequence and the cell barcode or complement thereof. In some instances, the dissociated cells or dissociated nuclei are partitioned with a particle, wherein at least some of the plurality of nucleic acid barcode molecules are immobilized on and/or in the particle. In some instances, the partition is a droplet. In some instances, the partition is a well. In some aspects, the partition is a well and wherein at least some of the plurality of nucleic acid barcode molecules are immobilized on a surface in the well.

In some aspects, the plurality of barcoded nucleic acid products or derivatives are subjected to sequencing. In some instances, a barcoded nucleic acid product or derivative of the plurality of barcoded nucleic acid products or derivatives comprises (i) a sequence of the composite barcode and (ii) the cell barcode. In some instances, a barcoded nucleic acid product or derivative of the plurality of barcoded nucleic acid products or derivatives comprises (i) the sequence of or associated with an analyte from the cell and (ii) the cell barcode.

In some instances, the plurality of nucleic acid barcode molecules are coupled to the particle and the method further comprises releasing the plurality of nucleic acid barcode molecules from the particle. In some cases, the plurality of nucleic acid barcode molecules is covalently coupled to the particle.

In some embodiments, the common cell barcode or complement thereof is appended to a plurality of analytes comprising a plurality of deoxyribonucleic acid (DNA) molecules, and the sequence of or associated with the analyte is a sequence of a DNA molecule of the plurality of DNA molecules. In some examples, the plurality of DNA molecules is generated from a plurality of ribonucleic acid molecules. In some instances, the common cell barcode or complement thereof is appended to a plurality of analytes comprising a plurality of mRNAs, and the sequence of or associated with the analyte is a sequence of a RNA molecule of the plurality of mRNAs. In some instances, the common cell barcode or complement thereof is appended to a plurality of analytes each bound by a labelling agent. In some cases, the labelling agent is coupled to a cell feature. In some instances, the labelling agent comprises an antibody or an epitope binding fragment thereof coupled to a reporter oligonucleotide, and the reporter oligonucleotide comprises the sequence of or associated with the analyte. In some examples, the analyte is an antigen receptor. In some examples, the analyte is a nucleic acid molecule comprising a V(D)J join comprising a V (variable) segment, a J (joint) segment, and optionally a D (diversity) segment between the V and J segments. In some instances, the analyte is a sequence of a perturbation agent or associated with a perturbation agent.

In some instances, a first probe molecule is directly or indirectly bound to the analyte prior to or during the single cell barcoding reaction. In some instances, the first probe molecule is hybridized to the analyte after dissociating the cells or nuclei. In some instances, the first probe molecule is hybridized to the analyte before dissociating the cells or nuclei.

In some embodiments, an extension reaction is performed using at least a portion of the first probe molecule to generate a barcoded nucleic acid product of the plurality of barcoded nucleic acid products. In some instances, the first probe is hybridized to the reporter oligonucleotide coupled to the labelling agent. In some instances, a second probe molecule is directly or indirectly bound to the analyte prior to or during the single cell barcoding reaction. In some instances, the second probe molecule is hybridized to the analyte after dissociating the cells or nuclei. In some instances, the second probe molecule is hybridized to the analyte before dissociating the cells or nuclei. In some instances, the first probe molecule and the second probe molecule are linked after hybridizing to the analyte or a derivative thereof. In some instances, the first probe molecule and the second probe molecule are linked by performing a ligation using the analyte or a splint molecule as template. In some instances, the first probe molecule hybridizes to a first sequence of the analyte and a second probe molecule hybridizes to a second sequence of the analyte and the first sequence and the second sequence of the analyte are separated by a gap of one or more nucleotides. In some instances, the gap between the first probe molecule and the second probe molecule hybridized to the first sequence and the second sequence is filled by performing an extension reaction using the analyte as template.

In some embodiments, the plurality of probes comprise a plurality of circular probes or a plurality of circularizable probes. In some instances, the circularizable probes are ligated to generate a plurality of circularized probe. In some instances, rolling circle amplification (RCA) is performed using the circularized probes as template to generate a plurality of RCA products in the plurality of cells. In some instances, detecting the two or more different barcode sequences of the composite barcode or complements thereof comprises detecting the two or more different barcode sequences or complements thereof in the RCA products generated in the plurality of cells.

In some instances, the single cell barcoding reaction comprises an extension reaction using at least a portion of the RCA product as template to generate an additional barcoded nucleic acid product of the plurality of barcoded nucleic acid products. In some instances, the additional barcoded nucleic acid product comprises the cell barcode and a complement of a sequence in the RCA product. In some instances, the RCA product comprises a sequence complementary to a capture sequence of a nucleic acid barcode molecule of the plurality of nucleic acid barcode molecules. In some instances, a plurality of amplification products each associated with a barcode sequence of the two or more different barcode sequences is detected, wherein the plurality of amplification products comprise a non-enzymatically amplified product. In some aspects, the plurality of amplification products is cleaved prior to the single cell barcoding reaction.

In some embodiments, the target binding sequence hybridizes to a transcript of a housekeeping gene or a derivative thereof (e.g., an mRNA transcript of the gene). In some instances, the target binding sequence hybridizes to a transcript of a commonly expressed gene or a derivative thereof expressed by at least 2 different cell types. In some instances, the target binding sequence hybridizes to a transcript of a commonly expressed gene or a derivative thereof expressed by at least 50% of the plurality of cells in the biological sample. In some instances, the commonly expressed gene is expressed in at least 50%, at least 60%, at least 70%, at least 80%, or at least 90% of cells in the biological sample. In some instances, the commonly expressed gene is expressed at a level of at least 5, 10, 20, 40, or 100 transcripts per cell. In some instances, the plurality of probes target a nuclear analyte. In some instances, the commonly expressed gene has a mean count of more than 20 transcripts per cell by single cell RNA sequencing. In some cases, the commonly expressed gene is expressed in at least 80% of cells in the biological sample. In some examples, the plurality of probes bind to a transcript of a target nucleic acid corresponding to a gene selected from the group consisting of: beta actin (ACTB), glyceraldeyde-3-phosphate dehydrogenase (GAPDH), Ubiquitin C (UBC), hypoxanthine guanine phosphoribosyl transferase (HPRT), succinate dehydrogenase complex, subunit A (SDHA) and Tyrosine 3-monooxygenase/tryptophan 5-monooxygenase activation protein, and zeta polypeptide (YWHAZ).

In some examples, the plurality of probes bind to a ribosomal protein. In some aspects, the plurality of probes bind to a plurality of molecules of ribosome small subunit (18S) ribosomal RNA (rRNA). In some instances, the target binding sequence binds to beta actin (ACTB).

In some instances, the plurality of cells is contacted with an additional plurality of probes, wherein the additional plurality of probes each comprises an additional target binding sequence and an additional barcode sequence, and wherein the additional target binding sequence in the additional plurality of probes are the same, and the additional barcode sequences in the additional plurality of probes are different. In some instances, the target binding sequence and the additional target binding sequence hybridizes two or more transcripts or derivatives thereof that are collectively expressed at a level of at least 5, 10, 20, 40, or 100 transcripts per cell. In some instances, the target binding sequence and the additional target binding sequence hybridizes to a plurality of transcripts or derivatives thereof that are collectively expressed in at least 50%, at least 60%, at least 70%, at least 80%, or at least 90% of cells in the biological sample.

In some embodiments, the composite barcode comprises at least 3, 4, 5, 6, 7, 8, 9 or 10 different barcode sequences. In some instances, the plurality of probes comprises at least 5, 10, 15, 20 or more separate probes comprising the same target binding sequence. In some instances, the plurality of probes comprises at least 10, 15, 20 or more different barcode sequences. In some instances, each circularizable probe of the plurality of circularizable probes is provided as a single molecule. In some instances, each circularizable probe of the plurality of circularizable probes is provided in two or more parts.

In some instances, detecting the two or more different barcode sequences or complements thereof comprises (a) binding a plurality of intermediate probes directly or indirectly to the barcode sequences or complements thereof, (b) binding a detectably labeled probes directly or indirectly to detection regions of the intermediate probes, and (c) detecting signals associated with the detectably labeled probes. In some instances, one or more wash steps are performed to remove unbound and/or nonspecifically bound intermediate probe molecules from the biological sample. In some aspects, detecting the two or more different barcode sequences or complements thereof comprises contacting the biological sample with a universal pool of detectably labeled probes and a first pool of intermediate probes, wherein the intermediate probes of the first pool of intermediate probes comprise hybridization regions complementary to the two or more different barcode sequences or complements thereof and reporter regions complementary to a detectably labeled probe of the universal pool of detectably labeled probes; detecting complexes formed between the two or more different barcode sequences or complements thereof, the intermediate probes of the first pool of intermediate probes, and the detectably labeled probes; and removing the intermediate probes of the first pool of intermediate probes and the detectably labeled probes. In some aspects, detecting the two or more different barcode sequences or complements thereof further comprises contacting the biological sample with the universal pool of detectably labeled probes and a second pool of intermediate probes, wherein the intermediate probes of the second pool of intermediate probes comprise hybridization regions complementary to the two or more different barcode sequences or complements thereof and reporter regions complementary to a detectably labeled probe of the universal pool of detectably labeled probes; and detecting complexes formed between the two or more different barcode sequences or complements thereof, the intermediate probes of the second pool of intermediate probes, and the detectably labeled probes.

In some aspects, each of the two or more different barcode sequences of the composite barcode is assigned a sequence of signal codes that identifies the barcode sequence and wherein detecting the two or more different barcode sequences or complements thereof comprises detecting the corresponding sequences of signal codes detected from sequential hybridization, detection, and removal of sequential pools of intermediate probes and the universal pool of detectably labeled probes. In some cases, the sequences of signal codes are fluorophore sequences assigned to the corresponding barcode sequences or complements thereof. In some embodiments, the detectably labeled probes are fluorescently labeled.

In some embodiments, detecting the two or more different barcode sequences or complements thereof comprises sequencing all or a portion of the two or more different barcode sequences or complements thereof. In some instances, the sequencing comprises determining a sequence of the rolling circle amplification product or products using sequencing-by-ligation (SBL) or sequencing-by-synthesis (SBS).

In some instances, a plurality of discrete cells are identified by performing cell segmentation. In some embodiments, cell segmentation comprises identifying nuclei of the discrete cells and/or identifying membrane of the discrete cells. In some embodiments, the composite barcode is associated with the location of the cell in the biological sample after detecting the two or more different barcode sequences of the composite barcode or complements thereof in the cell at a location in the biological sample (e.g., by imaging the biological sample). In some aspects, the cell barcode or complement thereof associates the composite barcode with the sequence of or associated with the analyte from the same single cell or nucleus.

In some instances, the biological sample is on a substrate during the contacting of the cells with the plurality of probes and during the detecting of the two or more different barcode sequences of the composite barcode or complements thereof. In some instances, the biological sample is a formalin-fixed sample. In some instances, the biological sample is a formalin-fixed, paraffin-embedded (FFPE) sample. In some instances, the biological sample is a fresh frozen sample.

In some embodiments, after detecting the two or more different barcode sequences of the composite barcode or complements thereof, the cells or nuclei are lysed or permeabilized to provide access to the cells or nuclei. In some instances, nuclei of the biological sample are dissociated. In some aspects, the biological sample is treated with a proteolytic enzyme. In some aspects, the biological sample is treated with a protease and/or a collagenase. In some aspects, the biological sample is physically disrupted. In some aspects, the biological sample is de-crosslinked.

In some aspects, the target binding sequence binds to a target DNA. In some instances, the target binding sequence binds to a target RNA. In some instances, the target binding sequence binds to an endogenous nucleic acid in the biological sample.

In some instances, the biological sample is imaged to detect the two or more different barcode sequences of the composite barcode or complements thereof. In some embodiments, the biological sample is stained prior to imaging the biological sample. In some examples, the biological sample is stained with a nuclear stain, a histological stain, and/or an immunologic stain. In some cases, the cell barcode and/or the composite barcode is associated with locations in the image of the biological sample.

Provided herein is a system comprising a plurality of probes, wherein the probes of the plurality of probes comprise a target binding sequence and a barcode sequence, and wherein the target binding sequences of the probes in the plurality of probes are the same, and the probes of the plurality of probes comprise different barcode sequences; reagents for performing a single cell barcoding reaction; and a dissociation buffer. In some embodiments, the reagents for performing the single cell barcoding reaction comprises a plurality of first probe molecules for binding a panel of target analytes.

In some embodiments, the dissociation buffer comprises a protease and/or a collagenase. In some aspects, the plurality of probes comprise a plurality of circularizable probes, and the system comprises one or more reagents for circularizing the plurality of circularizable probes. In some instances, the system comprises one or more reagents for generating a plurality of rolling circle amplification products (RCPs) using the circularizable probes. In some examples, a plurality of detectably labeled probes in the system each comprises a detectable label. In some cases, the system comprises a plurality of a sequencing primer, a plurality of detectably labeled nucleotides, and a polymerase. In some embodiments, the reagents for performing the single cell barcoding reaction comprises a plurality of nucleic acid barcode molecules. In some instances, the reagents for performing the single cell barcoding reaction comprises a first probe molecule and a second probe molecule for binding to a RCP of the plurality of RCPs generated using the circularizable probes.

### BRIEF DESCRIPTION OF THE DRAWINGS

The drawings illustrate certain features and advantages of this disclosure. These embodiments are not intended to limit the scope of the appended claims in any manner.

FIG. 1A shows a plurality of probes for generating a composite barcode in a cell of a plurality of cells, wherein a plurality of probes comprise the same target binding sequence (e.g., different molecules of the same mRNA target) and different barcode sequences (e.g., barcodes 1, 2, 3, . . . N). FIG. 1B shows an example method for barcoding cells of a tissue section and dissociating the cells for downstream analysis.

FIG. 2 schematically illustrates example labelling agents with nucleic acid molecules attached thereto.

FIG. 3 schematically shows an example of a barcode-carrying bead.

FIG. 4 illustrates an example of a barcode-carrying bead.

FIG. 5A schematically shows an example of labelling agents. FIG. 5B schematically shows an example workflow for processing nucleic acid molecules. FIG. 5C schematically shows an example workflow for processing nucleic acid molecules. FIG. 5D schematically shows an example workflow for processing nucleic acid molecules.

FIG. 6 shows an example of a microfluidic channel structure for partitioning individual analyte carriers.

FIG. 7 shows an example of a microfluidic channel structure for the controlled partitioning of beads into discrete droplets.

FIG. 8 shows an example of a microfluidic channel structure for delivering barcode carrying beads to droplets.

FIG. 9 schematically illustrates an example microwell array.

FIG. 10 schematically illustrates an example workflow for processing nucleic acid molecules.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

In some aspects, the present application provides new and improved methods, compositions, and systems for profiling biological targets (analytes) in a sample to determine a spatial location of the analytes (e.g., in a cell) in a biological sample. In some aspects, probes with barcodes are provided to generate a composite barcode and the barcodes are detected in situ to determine a spatial location before hybridizing probes to analytes (e.g., an RNA molecule) and performing sequencing.

While whole genome amplification and sequencing technologies provide genomic, transcriptomic, and/or proteomic profiling of analytes in cells, these techniques may not provide spatial information of the analytes. Therefore, there is a need for new and improved methods for analyzing various analytes using sequencing technologies that also provides information regarding their spatial locations in a biological sample. In some aspects, provided herein is a method of analyzing a biological sample, comprising: (a) contacting a plurality of cells in the biological sample with a plurality of probes to generate a composite barcode in a cell of the plurality of cells, wherein the plurality of probes each comprise a same target binding sequence and different barcode sequences; and wherein the composite barcode comprises two or more barcode sequences from two or more separate probe molecules of the plurality of probes; (b) detecting the two or more barcode sequences of the composite barcode or complements thereof from the cell at a location in the biological sample; (c) dissociating cells or nuclei from the biological sample; (d) performing single cell barcoding reaction to append a cell barcode or a complement thereof to i) a nucleic acid molecule comprising a barcode sequence of the different barcode sequences of the composite barcode, and ii) a sequence of or associated with an analyte from the cell, thereby generating a plurality of barcoded nucleic acid products; and (e) using the common cell barcode and location of composite barcode to locate the analyte in the biological sample. In some aspects, using the cell barcode and composite barcode preserves spatial information from the locations of the cells or nuclei in the biological sample and allows correlation of the spatial information to results from sequencing of a large number of analytes of the cells or nuclei.

In some in situ assays, probes (e.g., circularizable probes) are used to bind and detect analytes of interest. For generating a composite barcode in a cell to obtain spatial information, the probes provided herein are used to target genes that are expressed across different cell types with the goal of having multiple probes bind to a plurality of molecules (e.g., transcripts) to introduce a plurality of barcodes into a single cell or a single nucleus. In some aspects, the target of the plurality of probes provided herein can be any suitable nucleic acid (e.g., gene or transcript) that is present in at least a majority of cells in the biological sample and can be detected in situ at a location in the biological sample. For example, probes described herein are designed to target one or more housekeeping genes (e.g., an mRNA transcript of the genes).

### II. Composite Barcode Generation

In some embodiments, provided herein are a plurality of probes for contacting with a biological sample to generate a composite barcode in a cell or nucleus. In some aspects, for generating a composite barcode in a cell or nucleus to provide spatial information, the probes provided herein are used to target genes that are expressed across different cell types. In some aspects, a plurality of probes bind to highly expressed genes or transcripts of highly expressed genes. In some aspects, a plurality of probes bind to a plurality of transcripts in a single cell or a single nucleus to introduce a plurality of barcodes into the single cell or nucleus. The composite barcode or a complement thereof is detected in the cell at a location in the biological sample to provide spatial information.

In some aspects, the plurality of probes described herein are designed to target one or more housekeeping genes (e.g., an mRNA transcript of the genes). In some cases, the target binding sequence of the plurality of probes hybridizes to molecules of transcripts of a housekeeping gene. In some embodiments, a plurality of probes each comprise the same target binding sequence and different barcode sequences. In some aspects, the plurality of probes bind to transcript that is expressed by at least two different cell types. In some embodiments, the target binding sequence of the plurality of probes hybridizes a commonly expressed gene expressed by at least two different cell types. In some aspects, the plurality of probes bind to transcript that is expressed at a level of at least 5, 10, 20, 40, or 100 transcripts per cell. In some embodiments, the plurality of probes bind different target nucleic acids (e.g., transcripts) that collectively are expressed by multiple cell types and/or expressed at a level of at least 5, 10, 20, 40, or 100 transcripts per cell. In some embodiments, the plurality of probes bind different target nucleic acids (e.g., transcripts) expressed in at least 50%, at least 60%, at least 70%, at least 80%, or at least 90% of cells in the biological sample. In some embodiments, the plurality of probes bind target nucleic acids (e.g., transcripts) corresponding to a gene expressed a mean count of more than 20 transcripts per cell by single cell RNA sequencing. In some embodiments, the plurality of probes bind target nucleic acids (e.g., transcripts) expressed in at least 80% of cells in the biological sample. In some embodiments, the plurality of probes bind target nucleic acids (e.g., transcripts) corresponding to two or more genes collectively expressed in at least 80% of cells in the biological sample. In some embodiments, the plurality of probes bind target nucleic acids (e.g., transcripts) corresponding to a gene expressed at a similar expression level, wherein the expression level is at least 5, 10, 20, 40, or 100 transcripts per cell, in two or more cell types.

In some instances, the plurality of probes target nuclear genes. In some embodiments, the plurality of probes target ribosomal genes or a derivative thereof. In some instances, the plurality of probes target mitochondrial genes or a derivative thereof.

In some embodiments, the target binding sequence of the plurality of probes hybridizes a nucleic acid corresponding to a commonly expressed gene that has a mean count of more than 20 transcripts per cell by single cell RNA sequencing. In some embodiments, the target binding sequence of the plurality of probes hybridizes a transcript of a commonly expressed gene that has a mean count of more than 20 transcripts per cell by single cell RNA sequencing. In some cases, the biological sample is contacted with a plurality of probes that comprises two or more subsets of probes that target two or more different target nucleic acids, and the two or more different target nucleic acids are collectively are expressed at a level of at least 5, at least 10, at least 20, at least 40, or at least 100 transcripts per cell. In some embodiments, the two or more subsets of probes target two different sequences of the same target nucleic acid. For example, a first subset of probes targets a first target sequence of a transcript and a second subset of probes target a second target sequence of the same transcript. In some embodiments, the two or more subsets of probes target two different sequences of the same mRNA transcript.

In some instances, the plurality of cells are contacted with an additional plurality of probes, wherein the additional plurality of probes each comprises an additional target binding sequence and an additional barcode sequence, and wherein the additional target binding sequence in the additional plurality of probes are the same, and the additional barcode sequences in the additional plurality of probes are different. For example, the additional target binding sequence of the additional plurality of probes is different from the target binding sequence of the first plurality of probes.

In some embodiments, the target binding sequence of the plurality of probes hybridizes a nucleic acid corresponding to a commonly expressed gene that is expressed in at least 50%, at least 60%, at least 70%, at least 80%, or at least 90% of cells in the biological sample. In some instances, the commonly expressed gene is expressed in at least 80% of cells in the biological sample. In some aspects, a plurality of probes bind to a plurality of molecules of a single gene. In some aspects, a plurality of probes bind to a plurality of molecules of two or more different genes.

In some embodiments, the probes target a nucleic acid corresponding to one or more housekeeping genes e.g., genes used in Q-RT-PCR. In some embodiments, the target binding sequence binds to an endogenous nucleic acid in the biological sample.

In some embodiments, the probe targets a transcript of a gene that is expressed in a wide variety of cell types, such as genes commonly known as “housekeeping genes.” In some instances, the target binding sequence hybridizes to a housekeeping gene or a derivative thereof. In some examples, the probe targets a transcript of a gene selected from the group consisting of: beta actin (ACTB), glyceraldeyde-3-phosphate dehydrogenase (GAPDH), Ubiquitin C (UBC), hypoxanthine guanine phosphoribosyl transferase (HPRT), succinate dehydrogenase complex, subunit A (SDHA) and Tyrosine 3-monooxygenase/tryptophan 5-monooxygenase activation protein, and zeta polypeptide (YWHAZ). In some aspects, the target of the plurality of probes is a transcript of a gene corresponding to a ribosomal protein. In some aspects, the target of the plurality of probes is a transcript of a gene corresponding to a transcription factor (e.g., TATA-binding protein (TBP) or NF-κB). In some aspects, the target of the plurality of probes is associated with a transcription factor (e.g., TATA-binding protein (TBP) or NF-κB). In some instances, the plurality of probes bind to a ribosomal protein. In some aspects, a plurality of probes bind to a plurality of molecules of ribosome small subunit (18S) ribsosomal RNA (rRNA). In some cases, the target of the plurality of probes is selected based on the tissue sample type and/or the species of origin of the biological sample. In some cases, the plurality of probes bind to a plurality of molecules of ribosome small subunit (18S) ribsosomal RNA (rRNA). In some aspects, the target of the plurality of probes is a transcript of a gene corresponding to a cytoskeletal protein (e.g., a tubulin). In some aspects, the target of the plurality of probes is associated with a cytoskeletal protein (e.g., a tubulin). In some instances, the target binding sequence of a plurality of probes binds to molecules of beta actin (ACTB).

### A. Probes

In some aspects, the plurality of probes used to generate a composite barcode comprise a plurality of circular probes or circularizable probes. In some cases, a circularizable probe is provided in one or more parts (e.g., one or more separate nucleic acid molecules). In some instances, the plurality of probes comprise a plurality of circular probes. In some cases, a circularizable probe is provided in two or more parts (e.g., two or more separate nucleic acid molecules). A circularizable probe, in some embodiments, is any nucleic acid molecule or nucleic acid molecules that hybridizes to another one or more other nucleic acids such that the ends of the nucleic acid molecule or nucleic acid molecules are juxtaposed or are in proximity for ligation to form a circularized probe (e.g., by ligation with or without gap filling). For example, in some cases, a circularizable probe is a padlock probe with ends that can be ligated upon hybridization to a target nucleic acid sequence to form a circularized padlock probe. Specific probe designs can vary. In some instances, a probe described herein comprises a circularizable probe that does not require gap filling to circularize upon hybridization to a template (e.g., a target nucleic acid). In some instances, a probe described herein comprises a gapped circularizable probe (e.g., one that requires gap filling to circularize upon hybridization to a template).

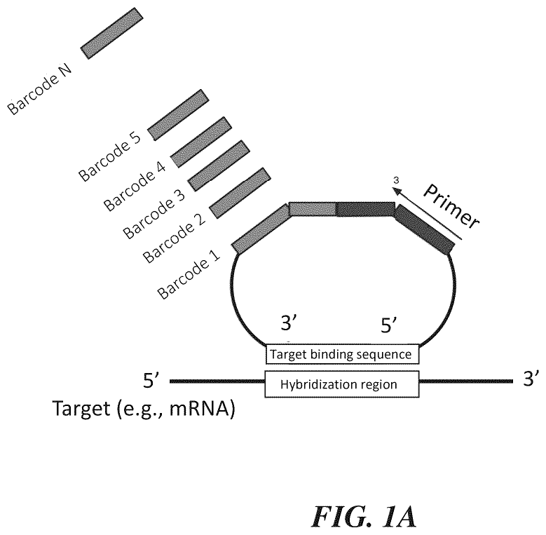

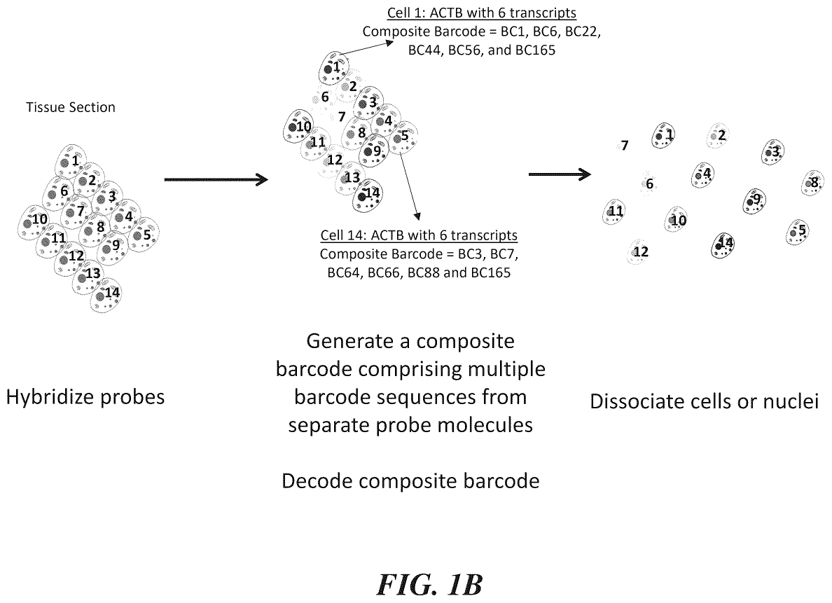

In some embodiments, the biological sample is contacted with a plurality of probes wherein the plurality of probes comprise at least 5, at least 10, at least 15, at least 20, at least 25, at least 30, at least 35, at least 40, or at least 50 different probes each comprising the same target binding sequence and different barcode sequence between each probe. In some embodiments, the plurality of probes comprises probes each comprising (i) a common target binding sequence and (ii) one ofat least 5, at least 10, at least 15, at least 20, at least 25, at least 30, at least 35, at least 40, or at least 50 different barcode sequences. In some embodiments, the biological sample is contacted with an additional plurality of probes wherein the additional plurality of probes comprise at least at least 5, at least 10, at least 15, at least 20, at least 25, at least 30, at least 35, at least 40, at least 50, at least 60, at least 70, at least 80, at least 90, at least 100, at least 120, or at least 150 different probes each comprising the same target binding sequence (e.g., to a different highly expressed gene) and different barcode sequence between each probe within the additional plurality of probes. For example, FIG. 1A shows a plurality of probes for generating a composite barcode in a cell of a plurality of cells, wherein a plurality of probes comprise the same target binding sequence (e.g., different molecules of the same mRNA target) and different barcode sequences (e.g., barcodes 1, 2, 3, . . . N). As shown in FIG. 1B, probes are contacted with cells of the tissue section, and different copies of the ACTB transcript in a first cell (e.g., Cell 1) are hybridized by 6 different probes, wherein the 6 different probes all bind to the same sequence in the ACTB transcript but the 6 different probes have 6 different barcodes that make up the composite barcode of Cell 1 (e.g., barcodes (BC) 1, BC6, BC22, BC44, BC56, and BC165). Different copies of the ACTB transcript in a second cell (e.g., Cell 14) are hybridized by 6 different probes. In Cell 14, 6 different barcodes make up the composite barcode of Cell 14 (e.g., BC3, BC7, BC64, BC66, BC88 and BC165). In some cases, the composite barcode generated in two different cells share the same barcode sequence (e.g., BC 165 in both Cell 1 and Cell 14).

In some embodiments, a circularizable probe is provided as a single nucleic acid molecule with ligatable 3′ and 5′ ends. In some cases, the circularizable probe is provided in two or more parts, e.g. two, three, four, five or more nucleic acid molecules, which create two or more ligation junctions upon hybridization to a target nucleic acid and/or splint. In some embodiments, the method comprises contacting the biological sample with a splint. In some embodiments, a first ligation junction is formed between a first and second part of a circularizable probe upon hybridization to a target nucleic acid, and a second ligation junction is formed between the first and second parts upon hybridization of the first and second probe to a splint. In some aspects, the parts provide (e.g. form or comprise) 3′ and/or 5′ ligatable ends which may be juxtaposed for ligation and be ligated. In some instances, the 5′ phosphate and 3′ hydroxyl are ligated together upon hybridization to a target nucleic acid sequence. In some embodiments, the probe comprise a target binding sequence that hybridizes to a target nucleic acid sequence in an endogenous nucleic acid (e.g., an endogenous DNA or an endogenous RNA molecule, optionally wherein the endogenous RNA is an mRNA). In some embodiments, the target nucleic acid sequence is in a probe bound directly or indirectly to an endogenous nucleic acid molecule (e.g., a DNA probe hybridized to an endogenous nucleic acid). In some cases, the target nucleic acid sequence is in a product of an endogenous molecule (e.g., an amplification product). For instance, in some embodiments, an RNA analyte is reverse transcribed to generate a DNA molecule, and the circularizable probe hybridizes to the DNA molecule. In some cases, the target nucleic acid sequence is in or is associated with a labelling agent that binds to a non-nucleic acid analyte. In some cases, the circularizable probe is provided as one or more DNA molecules. In some embodiments, the circularizable probe is or comprises a DNA/RNA chimera comprising one or more ribonucleotides. In some embodiments, the circularizable probe comprises one or more ribonucleotides at and/or near a ligatable 3′ end of the circularizable probe. In some embodiments, the circularizable probe comprises a ribonucleotide at its 3′ end. Various suitable circularizable probes are described in U.S. Pat. No. 11,597,965 (previously published as US Pat. Pub. 2020/0224244), the content of which is herein incorporated by reference in its entirety.

In some embodiments, a probe of the plurality of probes used to generate a composite barcode disclosed herein (e.g., a circularizable probe) comprises a 5′ flap which may be recognized by a structure-specific cleavage enzyme, e.g. an enzyme capable of recognizing the junction between single-stranded 5′ overhang and a DNA duplex, and cleaving the single-stranded overhang. It will be understood that the branched three-strand structure which is the substrate for the structure-specific cleavage enzyme may be formed by 5′ end of one probe part and the 3′ end of another probe part when both have hybridized to the target nucleic acid molecule, as well as by the 5′ and 3′ ends of a one-part probe. Enzymes suitable for such cleavage include Flap endonucleases (FENS), which are a class of enzymes having endonucleolytic activity and being capable of catalyzing the hydrolytic cleavage of the phosphodiester bond at the junction of single- and double-stranded DNA. Thus, in some embodiments, cleavage of the additional sequence 5′ to the first target-specific binding site is performed by a structure-specific cleavage enzyme, e.g. a Flap endonuclease. Suitable Flap endonucleases are described in Ma et al. 2000. JBC 275, 24693-24700 and in U.S. Pat. No. 11,597,965 (previously published as US Pat. Pub. 2020/022424), each of which is herein incorporated by reference in its entirety, and may include P. furiosus (Pfu), A. fulgidus (Afu), M. jannaschii (Mja) or M. thermoautotrophicum (Mth). In other embodiments an enzyme capable of recognizing and degrading a single-stranded oligonucleotide having a free 5′ end may be used to cleave an additional sequence (5′ flap) from a structure as described above. Thus, an enzyme having 5′ nuclease activity may be used to cleave a 5′ additional sequence. Such 5′ nuclease activity may be 5′ exonuclease and/or 5′ endonuclease activity. A 5′ nuclease enzyme is capable of recognizing a free 5′ end of a single-stranded oligonucleotide and degrading said single-stranded oligonucleotide. A 5′ exonuclease degrades a single-stranded oligonucleotide having a free 5′ end by degrading the oligonucleotide into constituent mononucleotides from its 5′ end. A 5′ endonuclease activity may cleave the 5′ flap sequence internally at one or more nucleotides. Further, a 5′ nuclease activity may take place by the enzyme traversing the single-stranded oligonucleotide to a region of duplex once it has recognized the free 5′ end, and cleaving the single-stranded region into larger constituent nucleotides (e.g. dinucleotides or trinucleotides), or cleaving the entire 5′ single-stranded region, e.g. as described in Lyamichev et al. (1999), PNAS 96, 6143-6148, which is herein incorporated by reference in its entirety, for Taq DNA polymerase and the 5′ nuclease thereof. Preferred enzymes having 5′ nuclease activity include Exonuclease VIII, or a native or recombinant DNA polymerase enzyme from Thermus aquaticus (Taq), Thermus thermophilus or Thermus flavus, or the nuclease domain therefrom.

In some embodiments, the circularizable probes each comprise one or more barcodes. In some aspects, a plurality of probes contacted with a biological sample each comprise the same target binding sequence and different barcode sequences. For example, probe A and probe B of a plurality of probes both comprise the same target binding sequence (e.g., binds to different molecules of the same target nucleic acid (e.g., RNA molecules), and probe A comprises barcode A while probe B comprises barcode B. In some embodiments, the barcode(s) of the probes are not indicative of (e.g., correspond to) the identity of an analyte in the biological sample. In some embodiments, a probe does not contain a barcode that is indicative of (e.g., correspond to) the identity of an analyte in the biological sample. In some aspects, different barcode sequences from two or more separate probe molecules of the plurality of probes are used to generate the composite barcode in a single cell or nucleus of the biological sample.

In some embodiments, the circularizable probe includes one or more barcode sequences. The barcode sequences, if present, may be of any length. If more than one barcode sequence is used, the barcode sequences may independently have the same or different lengths, such as at least 5, at least 10, at least 15, at least 20, at least 25, at least 30, at least 35, at least 40, at least 50 nucleotides in length. In some embodiments, the barcode sequence may be no more than 120, no more than 112, no more than 104, no more than 96, no more than 88, no more than 80, no more than 72, no more than 64, no more than 56, no more than 48, no more than 40, no more than 32, no more than 24, no more than 16, or no more than 8 nucleotides in length. Combinations of any of these are also possible, e.g., the barcode sequence may be between 5 and 10 nucleotides, between 8 and 15 nucleotides, etc.

The barcode sequence may be arbitrary or random. In certain cases, the barcode sequences are chosen so as to reduce or minimize homology with other components in a sample, e.g., such that the barcode sequences do not themselves bind to or hybridize with other nucleic acids suspected of being within the cell or other sample. In some embodiments, between a particular barcode sequence and another sequence (e.g., a cellular nucleic acid sequence in a sample or other barcode sequences in probes added to the sample), the homology may be less than 10%, less than 8%, less than 7%, less than 6%, less than 5%, less than 4%, less than 3%, less than 2%, or less than 1%. In some embodiments, the homology may be less than 20, less than 19, less than 18, less than 17, less than 16, less than 15, less than 14, less than 13, less than 12, less than 11, less than 10, less than 9, less than 8, less than 7, less than 6, less than 5, less than 4, less than 3, or less than 2 bases, and in some embodiments, the bases are consecutive bases.

In some aspects, the methods provided herein comprise contacting a biological sample with a plurality of probes (e.g., circular or circularizable probes) that hybridize to a target nucleic acid sequence in the biological sample probes to generate a composite barcode in a cell or nucleus, optionally, ligating the circularizable probes, and performing rolling circle amplification of the circular or circularized probes, and detecting the rolling circle amplification products at the locations of the cells or nuclei in the biological sample.

In some embodiments, the method comprise prior to the amplification, a step of removing molecules of the circular or circularizable probe that are not bound to a nucleic acid in the biological sample. In some instances, the method comprises ligating the ends of a circularizable probe hybridized to a nucleic acid to form a circularized probe.

In some aspects, the circularizable probes each comprise a target binding sequence wherein the target binding sequence of the probe is capable of hybridizing to a hybridization region of the target nucleic acid. In some aspects, the provided methods involve a step of contacting, or hybridizing the circularizable to a biological sample containing a target nucleic acid in order to form a hybridization complex. In some embodiments, the biological sample is a tissue sample. In some embodiments, the biological sample is a tissue slice or tissue section. In some embodiments, the biological sample is a fresh frozen tissue sample. In some embodiments, the biological sample is a paraffin embedded formalin fixed (FFPE) tissue sample. In some embodiments, the biological sample is permeabilized. In some embodiments, the method comprises decrosslinking and/or pre-permeabilizing the biological sample before contacting the biological sample with the plurality of probes. In some aspects, the biological sample is on a substrate. In some cases, a tissue sample is on the substrate.

In some aspects, binding refers to the coupling between two or more nucleic acids, e.g., oligonucleotides and/or polynucleotides. In some embodiments, the binding is indirect binding. In some embodiments, the binding is direct (e.g., binding comprising direct hybridization of nucleic acid sequences). The nature of the binding may vary. In some instances, a first nucleic acid sequence directly binds to a second nucleic acid sequence via hybridization of complementary sequences. In some instances, a first nucleic acid sequence indirectly binds to a second nucleic acid sequence via one or more intermediate nucleic acids. For example, an intermediate nucleic acid comprises a first region that binds to the first nucleic acid sequence and has a second region for binding to the second nucleic acid sequence, thereby forming a complex comprising the first and second nucleic acid sequences. In some embodiments, the hybridization comprises the pairing of substantially complementary or complementary nucleic acid sequences between the plurality of probes and corresponding target nucleic acids. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another. In some embodiments, the method comprises hybridizing the plurality of probes to target nucleic acid sequences at conditions optimized for hybridization of plurality of probes to fully complementary target nucleic acid sequences. In some cases, the method comprises performing one or more post-hybridization washes. In some embodiments, the one or more post-hybridization washes are performed under stringent conditions.

In some aspects, the provided methods comprise one or more steps of ligating a circularizable probe to form a circularized probe at one or more locations in the biological sample. In some embodiments, the ligation involves two or more ligations. In some embodiments, the ligation involves chemical ligation (e.g., click chemistry ligation). In some embodiments, the ligation involves template dependent ligation. In some embodiments, the ligation involves template independent ligation. In some embodiments, the ligation involves enzymatic ligation. In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together.

In some embodiments, the method further comprises prior to the circularizing step, a step of removing molecules of the circularizable probe that are not bound to a target nucleic acid in the biological sample. In some embodiments, the method comprises removing splint molecules not hybridized to the circularizable probe. In some embodiments, the method comprises ligating the ends of a circularizable probe hybridized to the target nucleic acid sequence to form a circularized probe. In some embodiments, the method comprises ligating the ends of a circularizable probe hybridized to a splint to form a circularized probe. In some embodiments, a first ligation is performed using the target nucleic acid sequence, and a second ligation is performed using a splint. In some embodiments, a 3′ end and a 5′ end of the circularizable probe is ligated using the target nucleic acid (e.g., RNA) as a template.

In some embodiments, the ligation involves chemical ligation (e.g., click chemistry ligation). In some embodiments, the chemical ligation involves template dependent ligation. In some embodiments, the chemical ligation involves template independent ligation. In some embodiments, the click reaction is a template-independent reaction (see, e.g., Xiong and Seela (2011), J. Org. Chem. 76(14): 5584-5597, which is herein incorporated by reference in its entirety). In some embodiments, the click reaction is a template-dependent reaction or template-directed reaction. In some embodiments, the template-dependent reaction is sensitive to base pair mismatches such that reaction rate is significantly higher for matched versus unmatched templates. In some embodiments, the click reaction is a nucleophilic addition template-dependent reaction. In some embodiments, the click reaction is a cyclopropane-tetrazine template-dependent reaction.

In some embodiments, the ligation involves enzymatic ligation. In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a direct ligation. In some embodiments, the ligation herein is an indirect ligation. In a direct ligation, the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, e.g., separated by one or more intervening nucleotides or “gaps.” In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, circularizable probe, or target nucleic acid. The gap may be a gap of 1 to 60 nucleotides or a gap of 1 to 40 nucleotides or a gap of 3 to 40 nucleotides. In specific embodiments, the gap may be a gap of about 1, about 2, about 3, about 4, about 5, about 6, about 7, about 8, about 9 or about 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, one or more wash steps are performed after ligating the circularizable probes to remove unbound probes. In some cases, the one or more wash steps is/are performed under stringent conditions. In some embodiments, the one or more wash steps is/are performed using phosphate buffered saline comprising a non-ionic surfactant. In some embodiments, the wash buffer is PBST.

### B. Amplification

In some embodiments, the methods disclosed herein comprise generating a plurality of amplification products comprising the different barcode sequences or complements thereof from the plurality of probes in the biological sample (e.g., in a cell or nucleus). In some embodiments, the amplification products comprise an enzymatically amplified product. In some embodiments, the amplification products comprise a non-enzymatically amplified product. In some embodiments, each generated amplification product comprise a barcode of the composite barcode of a single cell or nucleus. In some embodiments, the methods disclosed herein comprise performing rolling circle amplification using the plurality of probes described in Section II.A as template. In some embodiments, the generated amplification product comprises a barcode sequence of the composite barcode, and each single cell or single nuclei comprises a plurality of generated amplification products. In some embodiments, before dissociation of cells or nuclei of the biological sample, the barcode sequences or complements thereof in the amplification products generated in the plurality of cells are detected at a location in the biological sample. In some aspects, any suitable method for amplification (e.g., for generating multiple copies of the barcode sequences of the composite barcode) to aid in detection of the barcode sequences of the plurality of probes at corresponding locations in the biological sample can be used. In some instances, the complement of the barcode sequence in the probes are amplified and detected.

In some aspects, a plurality of probes (e.g., circular probes or circularized probes) are used as a template for amplification and a primer oligonucleotide is added to the biological sample for amplification. In some instances, the primer oligonucleotide is added with the plurality of probes. In some instances, the primer oligonucleotide is added before or after the plurality of probes are contacted with the sample. In some instances, the primer oligonucleotide for amplification of the circular probe or circularized probe comprises a sequence complementary to a target nucleic acid, as well as a sequence complementary to the probe that hybridizes to the target nucleic acid. In some embodiments, amplification of the plurality of probes is primed by the target nucleic acid. In some embodiments, a washing step is performed to remove any unbound probes, primers, etc. In some embodiments, the wash is a stringency wash. Washing steps can be performed at any point during the process to remove non-specifically bound probes, probes that have ligated, etc.

In some instances, a primer oligonucleotide for amplification of the plurality of probes comprises a single-stranded nucleic acid sequence having a 3′ end that can be used as a substrate for a nucleic acid polymerase in a nucleic acid extension reaction. The primer oligonucleotide can comprise both RNA nucleotides and DNA nucleotides (e.g., in a random or designed pattern). The primer oligonucleotide can also comprise other natural or synthetic nucleotides described herein that can have additional functionality. In some cases, the primer oligonucleotide is about 6 bases to about 100 bases, such as about 25 bases.

In some embodiments, amplification of the plurality of probes is primed by the target nucleic acids in the biological sample (e.g., target RNA). The target nucleic acid can optionally be immobilized in the biological sample. In some embodiments, the target RNA is cleaved by an enzyme (e.g., RNase H). In some embodiments, the target nucleic acid is cleaved at a position downstream of the sequences bound to the circularized probe. In some aspects, the methods disclosed herein allow targeting of RNase H activity to a particular region in a target RNA that is adjacent to or overlapping with a target sequence for a probe. For example, a nucleic acid oligonucleotide is designed to hybridize to a complementary oligonucleotide hybridization region in the target RNA. In some embodiments, a nucleic acid oligonucleotide is used to provide a DNA-RNA duplex for RNase H cleavage of the target RNA in the DNA-RNA duplex. In some embodiments, the oligonucleotide binds to the target RNA at a position that overlaps with the target sequence of the probe by about 1 to about 20 nucleotides or by about 8 to about 10 nucleotides. The cleaved target RNA itself can then be used to prime RCA of the plurality of probes (e.g., target-primed RCA). In some cases, a plurality of nucleic acid oligonucleotides can be used to perform target-primed RCA for a plurality of different target nucleic acids. In any of the embodiments herein, the RNase H comprises an RNase H1 and/or an RNAse H2. In some embodiments, RNase inactivating agents or inhibitors are added to the sample after cleaving the target RNA.

In some instances, upon addition of a DNA polymerase in the presence of appropriate dNTP precursors and other cofactors, the amplification primer is elongated by replication of multiple copies of the template. The amplification step can utilize isothermal amplification or non-isothermal amplification. In some embodiments, after the formation of the hybridization complex and any subsequent circularization (such as ligation of, e.g., a circularizable probe), rolling-circle amplification is used to generate a RCA product (e.g., RCP) containing multiple copies of the barcode sequence of the plurality of probes.

In some embodiments, RCPs are generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo−) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof. In some embodiments, the polymerase is Phi29 DNA polymerase.

In some embodiments, the polymerase comprises a modified recombinant Phi29-type polymerase. In some embodiments, the polymerase comprises a modified recombinant Phi29, B103, GA-1, PZA, Phi15, BS32, M2Y, Nf, G1, Cp-1, PRD1, PZE, SF5, Cp-5, Cp-7, PR4, PR5, PR722, or L17 polymerase. In some embodiments, the polymerase comprises a modified recombinant DNA polymerase having at least one amino acid substitution or combination of substitutions as compared to a wildtype Phi29 polymerase. Suitable polymerases are described in U.S. Pat. Nos. 8,257,954; 8,133,672; 8,343,746; 8,658,365; 8,921,086; and 9,279,155, all of which are herein incorporated by reference. In some embodiments, the polymerase is not directly or indirectly immobilized to a substrate, such as a bead or planar substrate (e.g., glass slide), prior to contacting a sample, although the sample may be immobilized on a substrate.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., at or about 27° C., at or about 29° C., at or about 31° C., at or about 33° C., at or about 35° C., at or about 37° C., at or about 39° C., at or about 41° C., at or about 43° C., at or about 45° C., at or about 47° C., or at or about 49° C.

In some aspects, during the amplification step, modified nucleotides are added to the reaction to incorporate the modified nucleotides in the amplification product (e.g., nanoball). Examples of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide reacts with an acrylic acid N-hydroxysuccinimide moiety. Examples of other amine-modified nucleotides comprise, but are not limited to, a 5-Aminoallyl-dUTP moiety modification, a 5-Propargylamino-dCTP moiety modification, a N⁶-6-Aminohexyl-dATP moiety modification, or a 7-Deaza-7-Propargylamino-dATP moiety modification. In some embodiments, the modified nucleotides comprises base modifications, such as azide and/or alkyne base modifications, dibenzylcyclooctyl (DBCO) modifications, vinyl modifications, trans-Cyclooctene (TCO), and so on.

In some embodiments, the extension reaction mixture comprises a deoxynucleoside triphosphate (dNTP) or derivative, variant, or analogue thereof. In some embodiments, the primer extension reaction mixture can comprise a catalytic cofactor of the polymerase. In any of the preceding embodiments, the primer extension reaction mixture can comprise a catalytic di-cation, such as Mg²⁺ and/or Mn²⁺.

In some aspects, the amplification product (e.g., RCA product) is anchored to a polymer matrix. The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products (e.g., RCA products) are copolymerized and/or covalently attached to the surrounding matrix thereby preserving their spatial relationship and any information inherent thereto. In some embodiments, the RCA products are generated from DNA or RNA within a cell embedded in the matrix. In some embodiments, the RCA products are functionalized to form covalent attachment to the matrix preserving their spatial information within the cell thereby providing a subcellular localization distribution pattern. In some embodiments, the provided methods involve embedding RCA products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing or probe hybridization while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

For example, a plurality of cells in a biological sample is contacted with a plurality of probes to generate a composite barcode in a cell of the plurality of cells, wherein the plurality of probes each comprise a same target binding sequence and different barcode sequences; wherein the composite barcode comprises multiple barcode sequences from two or more separate probe molecules of the plurality of probes; the probes are enzymatically or non-enzymatically amplified to generate an amplification product comprising multiple copies of a barcode sequence of the different barcode sequences, and detecting the multiple barcode sequences of the composite barcode or complements thereof in the generated amplification products in the cell at a location in the biological sample. In some instances, the generated amplification product is a RCA product, a hybridization chain reaction (HCR) product, a linear oligonucleotide hybridization chain reaction (LO-HCR) product, a branched DNA reaction (bDNA) product, or a primer exchange reaction (PER) product.

In some embodiments, the plurality of probes are detected by smFISH readout wherein a plurality of probes directly hybridize to multiple regions (e.g., sequences) of the same target transcript. In some embodiments, amplified signals associated with a plurality of probes are detected by using one or more detectably or non-detectably labeled probes (e.g., as in the case of a hybridization chain reaction (HCR), a branched DNA reaction (bDNA), or the like). In some instances, the disclosed methods may comprise the use of a branched DNA (bDNA) amplification approach. In branched DNA (bDNA) amplification, primary and secondary amplifier oligonucleotides, each containing multiple replicate binding sites, are assembled on, e.g., individual smFISH probes to form a branched structure which binds multiple copies of a fluorescently labeled probe (Xia, et al. (2019), “Multiplexed Detection of RNA Using MERFISH and Branched DNA Amplification”, Scientific Reports 9:7721, which is herein incorporated by reference in its entirety). The degree of amplification in bDNA amplification is controlled by the design of the amplification reaction, i.e., the assembled bDNA structures cannot grow indefinitely even in the presence of excess reagents, which may be used to control spot size or limit the variability in brightness from molecule to molecule (Xia, et al. (2019), ibid.).

Various suitable signal amplification methods include targeted deposition of detectable reactive molecules around the site of the probe hybridization, targeted assembly of branched structures (e.g., bDNA or branched assay using locked nucleic acid (LNA)), programmed in situ growth of concatemers by enzymatic rolling circle amplification (RCA) (e.g., as described in US 2019/0055594 incorporated herein by reference), hybridization chain reaction, assembly of topologically catenated DNA structures using serial rounds of chemical ligation (clampFISH), signal amplification via hairpin-mediated concatemerization (e.g., as described in U.S. Pat. No. 11,492,661, previously published as US Pat. Pub. 2020/0362398, which is herein incorporated by reference in its entirety), or primer exchange reactions such as signal amplification by exchange reaction (SABER). In some embodiments, a non-enzymatic signal amplification method may be used.

In some instances, a branched DNA (bDNA) amplification approach is used to detect the plurality of probes. In branched DNA (bDNA) amplification, primary and secondary amplifier oligonucleotides, each containing multiple replicate binding sites, are assembled on, e.g., individual smFISH probes to form a branched structure which binds multiple copies of a fluorescently labeled probe (Xia, et al. (2019), “Multiplexed Detection of RNA Using MERFISH and Branched DNA Amplification”, Scientific Reports 9:7721, which is herein incorporated by reference in its entirety). The degree of amplification in bDNA amplification is controlled by the design of the amplification reaction, i.e., the assembled bDNA structures cannot grow indefinitely even in the presence of excess reagents, which may be used to control spot size or limit the variability in brightness from molecule to molecule. In some embodiments, an amplification assembly complex comprises an amplifier hybridized directly or indirectly to the probe. In some embodiments, the assembly includes one or more amplifiers each including an amplifier repeating sequence. In some aspects, the one or more amplifiers is labeled. In some embodiments, the amplifier repeating sequence is about 5-30 nucleotides, and is repeated N times in the amplifier. In some embodiments, the amplifier repeating sequence is about 20 nucleotides, and is repeated at least two times in the amplifier. In some aspects, the one or more amplifier repeating sequence is labeled, see e.g., U.S. Pat. Pub. No. US20200399689A1, which is herein fully incorporated by reference in its entirety.

In some instances, the disclosed methods comprise the use of a hybridization chain reaction (HCR) approach to amplify signals. In a hybridization chain reaction, two fluorescently-labeled metastable hairpin oligonucleotides self-assemble into long fluorescent polymers starting from an initiator sequence present on each probe molecule (Xia, et al. (2019), ibid.). The degree of amplification achieved through HCR can be tuned by changing the hybridization or polymerization times, and can be adjusted to achieve highly amplified signals (which may, however, increase the size of the fluorescent spots generated and/or lead to variable degrees of amplification for different copies of the same target molecule).

In some embodiments, hybridization chain reaction (HCR) is used for amplification. HCR is an enzyme-free nucleic acid amplification based on a triggered chain of hybridization of nucleic acid molecules starting from HCR monomers, which hybridize to one another to form a nicked nucleic acid polymer. This polymer is the product of the HCR reaction which is ultimately detected in order to indicate the presence of the target analyte. HCR is described in detail in Dirks and Pierce, 2004, PNAS, 101(43), 15275-15278 and in U.S. Pat. No. 7,632,641 and US Pat. Pub. 2006/0234261, each of which is herein incorporated by reference in its entirety. HCR monomers typically comprise a hairpin, or other metastable nucleic acid structure. In the simplest form of HCR, two different types of stable hairpin monomer, referred to here as first and second HCR monomers, undergo a chain reaction of hybridization events to form a long nicked double-stranded DNA molecule when an “initiator” nucleic acid molecule is introduced. The HCR monomers have a hairpin structure comprising a double stranded stem region, a loop region connecting the two strands of the stem region, and a single stranded region at one end of the double stranded stem region. The single stranded region which is exposed (and which is thus available for hybridization to another molecule, e.g. initiator or other HCR monomer) when the monomers are in the hairpin structure may be known as the “toehold region” (or “input domain”). The first HCR monomers each further comprise a sequence which is complementary to a sequence in the exposed toehold region of the second HCR monomers. This sequence of complementarity in the first HCR monomers may be known as the “interacting region” (or “output domain”). Similarly, the second HCR monomers each comprise an interacting region (output domain), e.g. a sequence which is complementary to the exposed toehold region (input domain) of the first HCR monomers. In the absence of the HCR initiator, these interacting regions are protected by the secondary structure (e.g. they are not exposed), and thus the hairpin monomers are stable or kinetically trapped (also referred to as “metastable”), and remain as monomers (e.g. preventing the system from rapidly equilibrating), because the first and second sets of HCR monomers cannot hybridize to each other. However, once the initiator is introduced, it is able to hybridize to the exposed toehold region of a first HCR monomer, and invade it, causing it to open up. This exposes the interacting region of the first HCR monomer (e.g. the sequence of complementarity to the toehold region of the second HCR monomers), allowing it to hybridize to and invade a second HCR monomer at the toehold region. This hybridization and invasion in turn opens up the second HCR monomer, exposing its interacting region (which is complementary to the toehold region of the first HCR monomers), and allowing it to hybridize to and invade another first HCR monomer. The reaction continues in this manner until all of the HCR monomers are exhausted (e.g. all of the HCR monomers are incorporated into a polymeric chain). Ultimately, this chain reaction leads to the formation of a nicked chain of alternating units of the first and second monomer species. The presence of the HCR initiator is thus required in order to trigger the HCR reaction by hybridization to and invasion of a first HCR monomer. The first and second HCR monomers are designed to hybridize to one another are thus may be defined as cognate to one another. They are also cognate to a given HCR initiator sequence. HCR monomers which interact with one another (hybridize) may be described as a set of HCR monomers or an HCR monomer, or hairpin, system.

An HCR reaction could be carried out with more than two species or types of HCR monomers. For example, a system involving three HCR monomers could be used. In such a system, each first HCR monomer may comprise an interacting region which binds to the toehold region of a second HCR monomer; each second HCR may comprise an interacting region which binds to the toehold region of a third HCR monomer; and each third HCR monomer may comprise an interacting region which binds to the toehold region of a first HCR monomer. The HCR polymerization reaction would then proceed as described above, except that the resulting product would be a polymer having a repeating unit of first, second and third monomers consecutively.

In some embodiments, similar to HCR reactions that use hairpin monomers, linear oligo hybridization chain reaction (LO-HCR) can also be used for signal amplification. In some embodiments, provided herein is a method of detecting an analyte in a sample comprising: (i) performing a linear oligo hybridization chain reaction (LO-HCR), wherein an initiator is contacted with a plurality of LO-HCR monomers of at least a first and a second species to generate a polymeric LO-HCR product hybridized to a target nucleic acid molecule, wherein the first species comprises a first hybridization region complementary to the initiator and a second hybridization region complementary to the second species, wherein the first species and the second species are linear, single-stranded nucleic acid molecules; wherein the initiator is provided in one or more parts, and hybridizes directly or indirectly to or is comprised in the target nucleic acid molecule; and (ii) detecting the polymeric product, thereby detecting the analyte. In some embodiments, the first species and/or the second species may not comprise a hairpin structure. In some embodiments, the plurality of LO-HCR monomers may not comprise a metastable secondary structure. In some embodiments, the LO-HCR polymer may not comprise a branched structure. In some embodiments, performing the linear oligo hybridization chain reaction comprises contacting the target nucleic acid molecule with the initiator to provide the initiator hybridized to the target nucleic acid molecule

In some embodiments, amplification is achieved by performing a primer exchange reaction (PER). In various embodiments, a primer with domain on its 3′ end binds to a catalytic hairpin, and is extended with a new domain by a strand displacing polymerase. For example, a primer with domain 1 on its 3′ ends binds to a catalytic hairpin, and is extended with a new domain 1 by a strand displacing polymerase, with repeated cycles generating a concatemer of repeated domain 1 sequences. In various embodiments, the strand displacing polymerase is Bst. In various embodiments, the catalytic hairpin includes a stopper which releases the strand displacing polymerase. In various embodiments, branch migration displaces the extended primer, which can then dissociate. In various embodiments, the primer undergoes repeated cycles to form a concatemer primer. In various embodiments, a plurality of concatemer primers is contacted with the probe. see e.g., U.S. Pat. No. 11,286,517 (previously published as U.S. Pat. Pub. No. US20190106733), which is herein incorporated by reference in its entirety, for examples of molecules and PER reaction components.

In some embodiments, provided herein are methods and compositions for analyzing analytes in a sample using concatemer primers and labelling agents. In various embodiments, a primer with domain on its 3′ end binds to a catalytic hairpin, and is extended with a new domain by a strand displacing polymerase. For example, a primer with domain 1 on its 3 ends binds to a catalytic hairpin, and is extended with a new domain 1 by a strand displacing polymerase, with repeated cycles generating a concatemer of repeated domain 1 sequences. In various embodiments, the strand displacing polymerase is Bst. In various embodiments, the catalytic hairpin includes a stopper which releases the strand displacing polymerase. In various embodiments, branch migration displaces the extended primer, which can then dissociate. In various embodiments, the primer undergoes repeated cycles to form a concatemer primer.

In various embodiments, a plurality of concatemer primers is contacted with a sample. In various embodiments, an assembly include a plurality of concatemer primers, a plurality of labeled probes, and a sample including nucleic acids. In various embodiments, each the plurality of concatemer primers each includes domain 1, 2, 3, etc. In various embodiments, each the plurality of labeled probes each include domain 1′, 2′, 3′, etc., with each corresponding domain 1′, 2′, 3′ being complementary to domain 1, 2, 3, etc., respectively. In various embodiments, the assembly includes the plurality of concatemer primers, which are capable of hybridizing to target nucleic acid sequences in the sample. Described herein is a method using the aforementioned assembly, including contacting the sample including target nucleic acids with the plurality of concatemer primers, then contacting the sample and plurality of concatemer primers to generate an amplification product. See e.g., U.S. Pat. Pub. Nos. 2021/0147902 and 2020/0362398, each of which is herein incorporated by reference in its entirety.

### C. Detection

In some aspects, the methods disclosed herein comprise detecting the multiple barcode sequences of the composite barcode or complements thereof in the cell at a location in the biological sample. In some aspects, the location of the detected barcode sequences of the composite barcode or complements thereof in the biological sample is recorded. In some instances, barcode sequences in amplification products are detected. In some cases, the method comprise imaging the biological sample. In some embodiments, a plurality of images of the biological sample is acquired. For examples, one or more images of the biological sample stained with a nuclear stain, a histological stain, and/or an immunologic stain are acquired and one or more images are acquired to detect the multiple barcode sequences of the composite barcode or complements thereof in cells or nuclei at a location in the biological sample.

In some aspects, the methods provided herein comprise imaging a biological sample. In some instances, the biological sample is imaged prior to contacting the sample with the plurality of probes to generate a composite barcode. In some embodiments, the biological sample is imaged after contacting a plurality of cells in the biological sample with a plurality of probes to generate a composite barcode in a cell of the plurality of cells. In some embodiments, the biological sample is stained prior to imaging. In some embodiments, the biological sample is stained with a nuclear stain, a histological stain, and/or an immunologic stain. In some embodiments, the biological sample is stained with hematoxylin and eosin (H&E) stain. In some embodiments, the biological sample is stained with DAPI.

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. In some aspects, the sample is contacted with one or more labeled antibodies (e.g., a primary antibody specific for the analyte of interest and a labeled secondary antibody specific for the primary antibody). In some embodiments, cells in the sample are segmented using one or more images taken of the stained sample. In some aspects, detected different barcode sequences of the composite barcode are associated with segmented cells.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, ruthenium red, propidium iodide, rhodamine (e.g., rhodamine B), or safranine or derivatives thereof. In some embodiments, the sample may be stained with hematoxylin and eosin (H&E). In some embodiments, the biological sample is stained with DAPI. In some aspects, the DAPI stain is used to perform cell segmentation and the detected different barcode sequences of the composite barcode are associated with segmented cells.

DAPI (4′,6-diamidino-2-phenylindole) is a DNA-specific probe which forms a fluorescent complex by attaching in the minor grove of A-T rich sequences of DNA. It also forms nonfluorescent intercalative complexes with double-stranded nucleic acids. As DAPI can pass through an intact cell membrane, it can be used to stain both live and fixed cells. When bound to double-stranded DNA, DAPI has an absorption maximum at a wavelength of 358 nm (ultraviolet) and its emission maximum is at 461 nm (blue). Therefore, for fluorescence microscopy, DAPI is excited with ultraviolet light and is detected through a blue/cyan filter.

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples are destained. Any suitable methods of destaining or discoloring a biological sample may be utilized, and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, each of which is herein incorporated by reference in its entirety.

In some embodiments, the imaging is performed using a microscopy method such as bright field microscopy (e.g., to detect H&E staining), oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, the biological sample is imaged by confocal microscopy (for example, to detect immunofluorescent staining such as DAPI for identifying cell structures including nuclei, protein expression, or cell boundaries). Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data. In some embodiments, the biological sample is imaged by fluorescent microscopy and/or brightfield microscopy.

In some embodiments, detecting the barcode sequences or complements thereof comprises contacting the biological sample with detectably labeled probes. In some embodiments, the detecting comprises contacting the biological sample with one or more detectably labeled probes that directly or indirectly bind to amplification products (e.g., rolling circle amplification product). In some embodiments, the method comprises removing (e.g., dehybridizing) the one or more detectably-labeled probes from the rolling circle amplification product. In any of the embodiments herein, the contacting and removing (e.g., dehybridizing) steps can be repeated with the one or more detectably-labeled probes and/or one or more other detectably-labeled probes that directly or indirectly bind to the rolling circle amplification product.

In some embodiments, detecting the different barcode sequences of the composite barcode or complements thereof comprises binding intermediate probes directly or indirectly to the barcode sequences or complements thereof, binding a detectably labeled probes directly or indirectly to detection regions of the intermediate probes, and detecting signals associated with the detectably labeled probes. In some embodiments, the detectably labeled probes bind to one or more intermediate probes comprising one or more overhang regions (e.g., a 5′ and/or 3′ end of the probe that does not hybridize to the rolling circle amplification product). In some cases, the overhang region comprises a binding region for binding one or more detectably-labeled probes. In some embodiments, the detecting comprises contacting the biological sample with a pool of intermediate probes corresponding to different barcode sequences or portions thereof, and a pool of detectably-labeled probes corresponding to different detectable labels. In some embodiments, the biological sample is sequentially contacted with different pools of intermediate probes. In some instances, a common or universal pool of detectably-labeled probes is used in a plurality of sequential hybridization steps (e.g., with different pools of intermediate probes).

In some embodiments, detecting the different barcode sequences of the composite barcode or complements thereof comprises contacting the biological sample with a universal pool of detectably labeled probes and a first pool of intermediate probes, wherein the intermediate probes of the first pool of intermediate probes comprise hybridization regions complementary to the barcode sequences or complements thereof and reporter regions complementary to a detectably labeled probe of the universal pool of detectably labeled probes; detecting complexes formed between the barcode sequences or complements thereof, the intermediate probes of the first pool of intermediate probes, and the detectably labeled probes; and removing the intermediate probes of the first pool of intermediate probes and the detectably labeled probes.

In any of the embodiments herein, detecting the different barcode sequences of the composite barcode or complements thereof comprises contacting the biological sample with one or more intermediate probes that directly or indirectly bind to the rolling circle amplification product, wherein the one or more intermediate probes are detectable using one or more detectably-labeled probes. In some embodiments, the intermediate probe comprises: (i) a recognition sequence complementary to a barcode sequence or portion thereof in the rolling circle amplification product, and (ii) a binding site for the detectably labeled probe. In any of the embodiments herein, the detecting step can further comprise removing (e.g., dehybridizing) the one or more intermediate probes and/or the one or more detectably-labeled probes from the rolling circle amplification product. In any of the embodiments herein, the contacting and removing (e.g., dehybridizing) steps can be repeated with the one or more intermediate probes, the one or more detectably-labeled probes, one or more other intermediate probes, and/or one or more other detectably-labeled probes.

In some embodiments, fluorescence microscopy is used for detection and imaging of the detectably labeled probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, the different barcode sequences of the composite barcode or complements thereof is detected by sequencing. In some embodiments, sequencing is performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 5,750,341; 6,969,488; 6,172,218; and 6,306,597, each which is herein incorporated by reference in its entirety.

In some embodiments, detecting the different barcode sequences of the composite barcode or complements thereof comprises sequencing, using a base-by-base sequencing method, e.g., SBS or SBB. In some embodiments, the biological sample is contacted with a sequencing primer and base-by-base sequencing using a cyclic series of nucleotide incorporation or binding, respectively, thereby generating extension products of the sequencing primer is performed followed by removing, cleaving, or blocking the extension products of the sequencing primer.

Generally in sequencing-by-synthesis methods, a first population of detectably labeled nucleotides (e.g., dNTPs) are introduced to contact a template nucleotide (e.g., a barcode sequence in the RCP) hybridized to a sequencing primer, and a first detectably labeled nucleotide (e.g., A, T, C, or G nucleotide) is incorporated by a polymerase to extend the sequencing primer in the 5′ to 3′ direction using a complementary nucleotide (a first nucleotide residue) in the template nucleotide as template. A signal from the first detectably labeled nucleotide can then be detected. The first population of nucleotides may be continuously introduced, but in order for a second detectably labeled nucleotide to incorporate into the extended sequencing primer, nucleotides in the first population of nucleotides that have not incorporated into a sequencing primer are generally removed (e.g., by washing), and a second population of detectably labeled nucleotides are introduced into the reaction. Then, a second detectably labeled nucleotide (e.g., A, T, C, or G nucleotide) is incorporated by the same or a different polymerase to extend the already extended sequencing primer in the 5′ to 3′ direction using a complementary nucleotide (a second nucleotide residue) in the template nucleotide as template. Thus, in some embodiments, cycles of introducing and removing detectably labeled nucleotides are performed.

In some embodiments, the base-by-base sequencing comprises using a polymerase that is fluorescently labeled and one or more nucleotides that are not fluorescently labeled. In some embodiments, the base-by-base sequencing comprises using a polymerase-nucleotide conjugate comprising a fluorescently labeled polymerase linked to a nucleotide moiety that is not fluorescently labeled. In some embodiments, the base-by-base sequencing comprises using a multivalent polymer-nucleotide conjugate comprising a polymer core, multiple nucleotide moieties, and one or more fluorescent labels.

### III. Nucleic Acid Analysis

In some embodiments, after detecting the different barcode sequences of the composite barcode or complements thereof in the cell at a location in the biological sample (e.g., as described in Section II), cells or nuclei are dissociated from the biological sample and a single cell barcoding reaction is performed. For example, a cell barcode or a complement thereof is appended to a nucleic acid molecule comprising a barcode sequence of the different barcode sequences of the composite barcode, and to a sequence of or associated with an analyte of a plurality of analytes, thereby generating a plurality of barcoded nucleic acid products. In some embodiments, the plurality of barcoded nucleic acid products are generated using an extension reaction. In some embodiments, the plurality of barcoded nucleic acid products are generated using a ligation reaction. By using the appended cell barcode or a complement thereof, analytes from a single cell or single nucleus are associated with the location of the composite barcode in the biological sample. In some embodiments, the single cell reaction comprises partitioning cells or nuclei.

In some embodiments, the method comprises dissociating cells or nuclei from the biological sample before performing the single cell barcoding reaction. In some embodiments, the dissociating comprises treating the biological sample with an enzyme. In some instances, the enzyme used for dissociation of cells or nuclei is a protease or a collagenase such as Liberase). In some embodiments, the dissociating comprises physically disrupting the biological sample (e.g., to triturate the tissue). For example, to dissociate the cells (e.g., from a FFPE tissue sample), the tissue sample is placed in a tube with an enzyme mix (e.g., with protease and/or collagenase) and sheared with a pellet pestle. In some cases, the tissue sample and enzyme mixture is incubated and agitated by inversion or other physical disruption. In some instances, the tissue sample is triturated with a pipette and optionally aspirated and pushed through a needle to improve cell recovery. In some embodiments, the dissociated sample is washed and filtered. In some instances, to dissociate the cells (e.g., from a FFPE tissue sample), the tissue sample is placed in a tube with an enzyme mix (e.g., with protease and/or collagenase) and sheared using a programed dissociation protocol (e.g., using an Octo Dissociator). In some cases, the dissociated sample comprising cells is subject to cell staining and/or counting.

In some embodiments, the biological sample is incubated at a temperature of greater than 25° C., greater than 30° C., or greater than 35° C. for dissociation. In some embodiments, the biological sample is incubated at a temperature of about 37° C. for dissociation. In some embodiments, after dissociation of cells or nuclei from the biological sample, the sample is filtered (e.g., to remove debris).

In some embodiments, access to a nucleic acid molecule included in a cell may be provided by lysing or permeabilizing the cell. Lysing the cell may release the nucleic acid molecule contained therein from the cell. In some embodiments, cells of the biological sample are lysed using a lysis agent such as a bioactive agent. A bioactive agent useful for lysing a cell may be, for example, an enzyme (e.g., as described herein). An enzyme used to lyse a cell may or may not be capable of carrying out additional functions such as degrading, extending, reverse transcribing, or otherwise altering a nucleic acid molecule. Alternatively, an ionic or non-ionic surfactant such as TritonX-100, Tween 20, sarcosyl, or sodium dodecyl sulfate may be used to lyse a cell. Cell lysis may also be achieved using a cellular disruption method such as an electroporation or a thermal, acoustic, or mechanical disruption method. Alternatively, a cell may be permeabilized to provide access to a nucleic acid molecule included therein. Permeabilization may involve partially or completely dissolving or disrupting a cell membrane or a portion thereof. Permeabilization may be achieved by, for example, contacting a cell membrane with an organic solvent (e.g., methanol) or a detergent such as Triton X-100 or NP-40.

### A. Analytes

In some embodiments, a single cell barcoding reaction is performed to analyze a plurality of analytes of the cells of the biological sample. In some embodiments, a single cell barcoding reaction is performed to analyze different types of analytes in the biological sample. In some instances In some embodiments, a single cell barcoding reaction is performed to analyze at least two different types of analytes (e.g., proteins and RNA transcripts). In some embodiments, a single cell barcoding reaction is performed to analyze gene expression in the cells of the biological sample. In some aspects, the methods provided herein comprises the use of a targeting process to, e.g., enrich selected nucleic acid molecules within a sample. For example, target enrichment comprises providing a plurality of barcoded nucleic acid molecules and hybridizing barcoded nucleic acid molecules comprising targeted regions of interest to oligonucleotide probes (“baits”) which are complementary to the targeted regions of interest (or to regions near or adjacent to the targeted regions of interest). Baits may be attached to a capture molecule, including without limitation a biotin molecule. The capture molecule (e.g., biotin) can be used to selectively pull down the targeted regions of interest (for example, with magnetic streptavidin beads) to thereby enrich the resultant population of barcoded nucleic acid molecules for those containing the targeted regions of interest. Another exemplary enrichment method may comprise providing a plurality of barcoded nucleic acid molecules comprising a plurality of different barcode sequences, identifying a barcode sequence of the plurality of different barcode sequences, and enriching barcoded nucleic acid molecules comprising the barcode sequence. In some instances, enrichment comprises performing a nucleic acid extension reaction using a barcoded nucleic acid molecule comprising the barcode sequence and a primer comprising a sequence specific for the barcode sequence to generate an enriched plurality of barcoded nucleic acid molecules comprising the barcode sequence of interest. Details of such processes and additional schemes are included in, for example, International Patent Application No. PCT/US2020/012413, U.S. Pat. Pub. US2022/0025435, and U.S. Pat. No. 11,000,049, each of which is herein incorporated by reference in its entirety.

The present disclosure provides methods and systems for multiplexing, and otherwise increasing throughput in, analysis. For example, a single or integrated process workflow may permit the processing, identification, and/or analysis of more or multiple analytes, more or multiple types of analytes, and/or more or multiple types of analyte characterizations. For example, in the methods and systems described herein, one or more labelling agents capable of binding to or otherwise coupling to one or more cell features may be used to characterize biological particles and/or cell features. In some instances, cell features include cell surface features. Cell surface features may include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof. A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have a first reporter oligonucleotide coupled thereto, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969, each of which is herein incorporated by reference in its entirety.

In some aspects, the labelling agents bind to the analyte after dissociating the cells or nuclei. In some aspects, labelling agents bind to the analyte before dissociating the cells or nuclei. In some aspects, labelling agents are contacted with the biological sample prior to contacting the biological samples with the plurality of probes to generate the composite barcode. In some aspects, labelling agents are contacted with the biological sample after contacting the biological samples with the plurality of probes to generate the composite barcode. In some embodiments, cells in the sample is segmented using one or more detected labelling agents at locations in the biological sample.

In a particular example, a library of potential cell feature labelling agents may be provided, where the respective cell feature labelling agents are associated with nucleic acid reporter molecules, such that a different reporter oligonucleotide sequence is associated with each labelling agent capable of binding to a specific cell feature. In some aspects, different members of the library may be characterized by the presence of a different oligonucleotide sequence label. For example, an antibody capable of binding to a first protein may have associated with it a first reporter oligonucleotide sequence, while an antibody capable of binding to a second protein may have a different reporter oligonucleotide sequence associated with it. The presence of the particular oligonucleotide sequence may be indicative of the presence of a particular antibody or cell feature which may be recognized or bound by the particular antibody.

Labelling agents capable of binding to or otherwise coupling to one or more biological particles may be used to characterize a biological particle as belonging to a particular set of biological particles. For example, labelling agents may be used to label a sample of cells or a group of cells. In this way, a group of cells may be labeled as different from another group of cells. In an example, a first group of cells may originate from a first sample and a second group of cells may originate from a second sample. Labelling agents may allow the first group and second group to have a different labelling agent (or reporter oligonucleotide associated with the labelling agent). This may, for example, facilitate multiplexing, where cells of the first group and cells of the second group may be labeled separately and then pooled together for downstream analysis. The downstream detection of a label may indicate analytes as belonging to a particular group.

For example, a reporter oligonucleotide may be linked to an antibody or an epitope binding fragment thereof, and labeling a biological particle may comprise subjecting the antibody-linked barcode molecule or the epitope binding fragment-linked barcode molecule to conditions suitable for binding the antibody to a molecule present on a surface of the biological particle. The binding affinity between the antibody or the epitope binding fragment thereof and the molecule present on the surface may be within a desired range to ensure that the antibody or the epitope binding fragment thereof remains bound to the molecule. For example, the binding affinity may be within a desired range to ensure that the antibody or the epitope binding fragment thereof remains bound to the molecule during various sample processing steps, such as partitioning and/or nucleic acid amplification or extension. A dissociation constant (Kd) between the antibody or an epitope binding fragment thereof and the molecule to which it binds may be less than any of about 100 μM, about 90 μM, about 80 μM, about 70 μM, about 60 μM, about 50 μM, about 40 μM, about M, about 20 μM, about 10 μM, about 9 μM, about 8 μM, about 7 μM, about 6 μM, about 5 μM, about 4 μM, about 3 μM, about 2 μM, about 1 μM, about 900 nM, about 800 nM, about 700 nM, about 600 nM, about 500 nM, about 400 nM, about 300 nM, about 200 nM, about 100 nM, about 90 nM, about 80 nM, about 70 nM, about 60 nM, about 50 nM, about 40 nM, about 30 nM, about 20 nM, about 10 nM, about 9 nM, about 8 nM, about 7 nM, about 6 nM, about 5 nM, about 4 nM, about 3 nM, about 2 nM, about 1 nM, about 900 μM, about 800 μM, about 700 μM, about 600 μM, about 500 μM, about 400 μM, about 300 μM, about 200 μM, about 100 μM, about 90 μM, about 80 μM, about 70 μM, about 60 μM, about 50 μM, about 40 μM, about 30 μM, about 20 μM, about 10 μM, about 9 μM, about 8 μM, about 7 μM, about 6 μM, about 5 μM, about 4 μM, about 3 μM, about 2 μM, or about 1 μM. For example, the dissociation constant may be less than about 10 μM.

In another example, a reporter oligonucleotide may be coupled to a cell-penetrating peptide (CPP), and labeling cells may comprise delivering the CPP coupled reporter oligonucleotide into an biological particle. Labeling biological particles may comprise delivering the CPP conjugated oligonucleotide into a cell and/or cell bead by the cell-penetrating peptide. A cell-penetrating peptide that can be used in the methods provided herein can comprise at least one non-functional cysteine residue, which may be either free or derivatized to form a disulfide link with an oligonucleotide that has been modified for such linkage. Non-limiting examples of cell-penetrating peptides that can be used in embodiments herein include penetratin, transportan, plsl, TAT(48-60), pVEC, MTS, and MAP. Cell-penetrating peptides useful in the methods provided herein can have the capability of inducing cell penetration for at least about 30%, at least about 40%, at least about 50%, at least about 60%, at least about 70%, at least about 80%, at least about 90%, at least about 95%, at least about 96%, at least about 97%, at least about 98%, at least about 99%, or about 100% of cells of a cell population. The cell-penetrating peptide may be an arginine-rich peptide transporter. The cell-penetrating peptide may be Penetratin or the Tat peptide.

In another example, a reporter oligonucleotide may be coupled to a fluorophore or dye, and labeling cells may comprise subjecting the fluorophore-linked barcode molecule to conditions suitable for binding the fluorophore to the surface of the biological particle. In some instances, fluorophores can interact strongly with lipid bilayers and labeling biological particles may comprise subjecting the fluorophore-linked barcode molecule to conditions such that the fluorophore binds to or is inserted into a membrane of the biological particle. In some cases, the fluorophore is a water-soluble, organic fluorophore. In some instances, the fluorophore is Alexa 532 maleimide, tetramethylrhodamine-5-maleimide (TMR maleimide), BODIPY-TMR maleimide, Sulfo-Cy3 maleimide, Alexa 546 carboxylic acid/succinimidyl ester, Atto 550 maleimide, Cy3 carboxylic acid/succinimidyl ester, Cy3B carboxylic acid/succinimidyl ester, Atto 565 biotin, Sulforhodamine B, Alexa 594 maleimide, Texas Red maleimide, Alexa 633 maleimide, Abberior STAR 635P azide, Atto 647N maleimide, Atto 647 SE, or Sulfo-Cy5 maleimide. See, e.g., Hughes L D, et al. PLoS One. 2014 Feb. 4; 9(2):e87649, which is herein incorporated by reference in its entirety, for a description of organic fluorophores.

A reporter oligonucleotide may be coupled to a lipophilic molecule, and labeling biological particles may comprise delivering the nucleic acid barcode molecule to a membrane of the biological particle or a nuclear membrane by the lipophilic molecule. Lipophilic molecules can associate with and/or insert into lipid membranes such as cell membranes and nuclear membranes. In some cases, the insertion can be reversible. In some cases, the association between the lipophilic molecule and biological particle may be such that the biological particle retains the lipophilic molecule (e.g., and associated components, such as nucleic acid barcode molecules, thereof) during subsequent processing (e.g., partitioning, cell permeabilization, amplification, pooling, etc.). The reporter nucleotide may enter into the intracellular space and/or a cell nucleus.

A reporter oligonucleotide may be part of a nucleic acid molecule comprising any number of functional sequences, as described elsewhere herein, such as a target capture sequence, a random primer sequence, and the like, and coupled to another nucleic acid molecule that is, or is derived from, the analyte.

In some embodiments, prior to partitioning, the cells are incubated with the library of labelling agents, that may be labelling agents to a broad panel of different cell features, e.g., receptors, proteins, etc., and which include their associated reporter oligonucleotides. Unbound labelling agents may be washed from the cells, and the cells may then be co-partitioned (e.g., into droplets or wells) along with partition-specific barcode oligonucleotides (e.g., attached to a support, such as a bead or gel bead) as described elsewhere herein. As a result, the partitions may include the cell or cells, as well as the bound labelling agents and their known, associated reporter oligonucleotides.

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide. For example, the first plurality of the labelling agent and second plurality of the labelling agent may interact with different cells, cell populations or samples, allowing a particular report oligonucleotide to indicate a particular cell population (or cell or sample) and cell feature. In this way, different samples or groups can be independently processed and subsequently combined together for pooled analysis (e.g., partition-based barcoding as described elsewhere herein). See, e.g., U.S. Pat. Pub. US2019/0323088, which is herein incorporated by reference in its entirety.

As described elsewhere herein, libraries of labelling agents may be associated with a particular cell feature as well as be used to identify analytes as originating from a particular biological particle, population, or sample. The biological particles may be incubated with a plurality of libraries and a given biological particle may comprise multiple labelling agents. For example, a cell may comprise coupled thereto a lipophilic labelling agent and an antibody. The lipophilic labelling agent may indicate that the cell is a member of a particular cell sample, whereas the antibody may indicate that the cell comprises a particular analyte. In this manner, the reporter oligonucleotides and labelling agents may allow multi-analyte, multiplexed analyses to be performed.

In some instances, these reporter oligonucleotides comprise nucleic acid barcode sequences that permit identification of the labelling agent which the reporter oligonucleotide is coupled to. The use of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents may be achieved through any of a variety of direct or indirect, covalent or non-covalent associations or attachments. For example, oligonucleotides may be covalently attached to a portion of a labelling agent (such a protein, e.g., an antibody or antibody fragment), e.g., via a linker, using chemical conjugation techniques (e.g., LIGHTNING-LINK® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is herein incorporated by reference in its entirety. Likewise, protein and peptide biotinylation techniques have been developed and are readily available. See, e.g., U.S. Pat. No. 6,265,552, which is herein incorporated by reference in its entirety. Furthermore, click reaction chemistry such as a Methyltetrazine-PEG5-NHS Ester reaction, a TCO-PEG4-NHS Ester reaction, or the like, may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labelling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to an oligonucleotide that is complementary to a sequence of the reporter oligonucleotide, and the oligonucleotide may be allowed to hybridize to the reporter oligonucleotide.

In some embodiments, the single cell barcoding reaction is performed to append a cell barcode or a complement thereof to a plurality of analytes or derivatives thereof. In some instances, the plurality of analytes or derivatives thereof include a plurality of deoxyribonucleic acid (DNA) molecules. In some embodiments, the single cell barcoding reaction is performed to append a cell barcode or a complement thereof to a plurality of nucleic acid analytes directly or indirectly. In some embodiments, the single cell barcoding reaction is performed to append a cell barcode or a complement thereof to a plurality of DNA molecules. In some embodiments, the plurality of analytes comprise a plurality of mRNAs. In some embodiments, the plurality of analytes is bound by a plurality of labelling agent. For example, the labelling agent is coupled to a cell feature. In some embodiments, the labelling agent comprises an antibody or an epitope binding fragment thereof coupled to a reporter oligonucleotide. In some instances, the plurality of analytes comprise an antigen receptor. In some instances, the plurality of analytes comprise a nucleic acid molecule comprising a V(D)J join comprising a V (variable) segment, a J (joint) segment, and optionally a D (diversity) segment between the V and J segments. In some instances, the plurality of analytes comprise a sequence of a perturbation agent or associated with a perturbation agent.

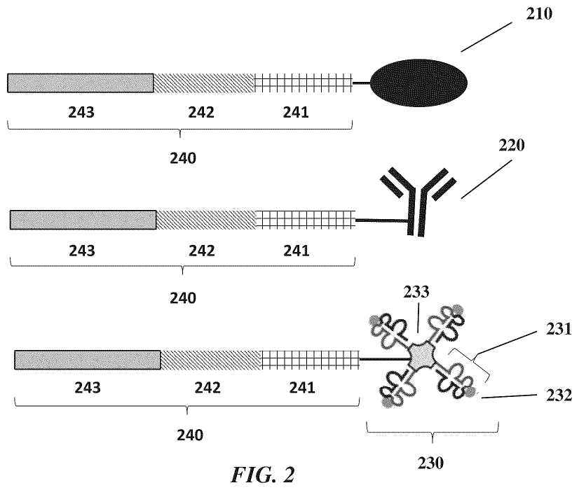

FIG. 2 depicts examples of labelling agents (210, 220, 230) comprising reporter oligonucleotides (240) attached thereto. Labelling agent 210 (e.g., a labeling agent selected from any of the labelling agents described herein) is attached (either directly, e.g., covalently attached, or indirectly) to reporter oligonucleotide 240. Reporter oligonucleotide 240 may comprise barcode sequence 242 that identifies labelling agent 210. Reporter oligonucleotide 240 may also comprise one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, or a sequencing primer or primer biding sequence (such as an R1, R2, or partial R1 or R2 sequence).

Referring to FIG. 2, in some instances, reporter oligonucleotide 240 conjugated to a labelling agent (e.g., 210, 220, 230) comprises a functional sequence 241 (e.g., a primer sequence), a barcode sequence that identifies the labelling agent (e.g., 210, 220, 230), and functional sequence 243. Functional sequence 243 can be a reporter capture handle sequence configured to hybridize to a complementary sequence, such as a complementary sequence present on a nucleic acid barcode molecule (not shown), such as those described elsewhere herein. In some instances, nucleic acid barcode molecule is attached to a support (e.g., a bead, such as a gel bead), such as those described elsewhere herein (e.g., in Section III.B.). In some embodiments, nucleic acid barcode molecule is attached to the support via a releasable linkage (e.g., comprising a labile bond), such as those described elsewhere herein. In some instances, reporter oligonucleotide 240 comprises one or more additional functional sequences, such as those described above.

In some instances, the labelling agent 210 is a protein or polypeptide (e.g., an antigen or prospective antigen) comprising reporter oligonucleotide 240. Reporter oligonucleotide 240 comprises barcode sequence 242 that identifies polypeptide 210 and can be used to infer the presence of an analyte, e.g., a binding partner of polypeptide 210 (i.e., a molecule or compound to which polypeptide 210 can bind). In some instances, the labelling agent 210 is a lipophilic moiety (e.g., cholesterol) comprising reporter oligonucleotide 240, where the lipophilic moiety is selected such that labelling agent 210 integrates into a membrane of a cell or nucleus. Reporter oligonucleotide 240 comprises barcode sequence 242 that identifies lipophilic moiety 210 which in some instances is used to tag cells (e.g., groups of cells, cell samples, etc.) and may be used for multiplex analyses as described elsewhere herein. In some instances, the labelling agent is an antibody 220 (or an epitope binding fragment thereof) comprising reporter oligonucleotide 240. Reporter oligonucleotide 240 comprises barcode sequence 242 that identifies antibody 220 and can be used to infer the presence of, e.g., a target of antibody 220 (i.e., a molecule or compound to which antibody 220 binds). In other embodiments, labelling agent 230 comprises an MHC molecule 231 comprising peptide 232 and reporter oligonucleotide 240 that identifies peptide 232. In some instances, the MHC molecule is coupled to a support 233. In some instances, support 233 may be a polypeptide, such as streptavidin, or a polysaccharide, such as dextran. In some instances, reporter oligonucleotide 240 may be directly or indirectly coupled to MHC labelling agent 230 in any suitable manner. For example, reporter oligonucleotide 240 may be coupled to MHC molecule 231, support 233, or peptide 232. In some embodiments, labelling agent 230 comprises a plurality of MHC molecules, (e.g. is an MHC multimer, which may be coupled to a support (e.g., 233)). There are many possible configurations of Class I and/or Class II MHC multimers that can be utilized with the compositions, methods, and systems disclosed herein, e.g., MHC tetramers, MHC pentamers (MHC assembled via a coiled-coil domain, e.g., Pro5@MHC Class I Pentamers, (ProImmune, Ltd.), MHC octamers, MHC dodecamers, MHC decorated dextran molecules (e.g., MHC Dextramer® (Immudex)), etc. For a description of exemplary labelling agents, including antibody and MHC-based labelling agents, reporter oligonucleotides, and methods of use, see, e.g., U.S. Pat. No. 10,550,429 and U.S. Pat. Pub. 20190367969, each of which is herein entirely incorporated by reference for all purposes.

### B. Barcoding

In some embodiments, a single cell barcoding reaction is performed using a plurality of nucleic acid barcode molecules covalently coupled to a particle (e.g., a bead). In some embodiments, a single cell barcoding reaction is performed using barcode carrying beads. In some embodiments, a single cell barcoding reaction is performed by partitioning cells or nuclei of a biological sample.

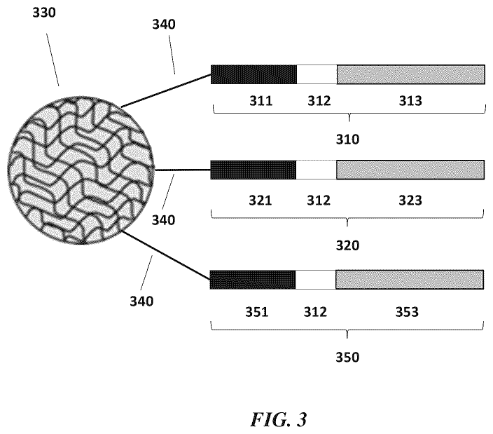

For example, FIG. 3 illustrates an example of a barcode carrying bead. In some embodiments, a cell barcode or a complement thereof is appended to a nucleic acid molecule comprising at least one barcode sequence of the different barcode sequences of the composite barcode and cell barcodes are appended to multiple analytes (e.g., RNA and one or more analytes using labelling agents described herein). In some aspects, an example of nucleic acid barcode molecules are generally depicted in FIG. 3. In some embodiments, nucleic acid barcode molecules 310 and 320 are attached to support 330 via a releasable linkage 340 (e.g., comprising a labile bond) as described elsewhere herein. In some embodiments, the nucleic acid barcode molecule 310 comprises an adapter sequence 311, barcode sequence (e.g., a cell barcode sequence) 312 and capture sequence 313. Nucleic acid barcode molecule 320 may comprise adapter sequence 321, barcode sequence (e.g., the cell barcode sequence) 312, and capture sequence 323, wherein a first capture sequence 323 comprises a different sequence than a second capture sequence 313. In some instances, adapter 311 and adapter 321 comprise the same sequence. In some instances, adapter 311 and adapter 321 comprise different sequences. Although support 330 is shown comprising nucleic acid barcode molecules 310 and 320, any suitable number of barcode molecules comprising common barcode sequence 312 are contemplated herein. For example, in some embodiments, support 330 further comprises nucleic acid barcode molecule 350. Nucleic acid barcode molecule 350 may comprise adapter sequence 351, barcode sequence (e.g., the cell barcode sequence) 312 and capture sequence 353, wherein capture sequence 353 comprises a different sequence than capture sequence 313 and 323. In some aspects, a single particle (bead) comprises two or more capture sequences (e.g., a first capture sequence 313 for hybridizing to an analyte such as an mRNA and a second capture sequence 323 for hybridizing to a nucleic acid molecule (e.g., a cleaved portion of an RCP detected in the biological sample) comprising a sequence of the composite barcode. In some instances, an amplification products, e.g., a RCA product, comprises a sequence complementary to a capture sequence of a nucleic acid barcode molecule. In some instances, nucleic acid barcode molecules (e.g., 310, 320, 350) comprise one or more additional functional sequences, such as a UMI or other sequences described herein. The nucleic acid barcode molecules 310, 320 or 350 may interact with analytes (e.g., as described in Section III.A.) or a nucleic acid molecule comprising a barcode sequence of the composite barcode (e.g., as described in Section II).

### Nucleic Acid Barcode Molecules

In some embodiments, a nucleic acid barcode molecule comprises one or more barcode sequences. In some embodiments, a plurality of nucleic acid barcode molecules are coupled to a particle (e.g., a bead). In some embodiments, the one or more barcode sequences include sequences that are the same for all or a portion of the nucleic acid molecules coupled to a given bead and/or sequences that are different across all (or a portion of the) nucleic acid molecules coupled to the given bead. The nucleic acid molecule may be incorporated into the bead.

In some aspects, nucleic acid barcode molecules comprise one or more functional sequences for coupling to an analyte, another nucleic acid molecule or analyte tag such as a reporter oligonucleotide. Such functional sequences can include, e.g., a template switch oligonucleotide (TSO) sequence, a primer sequence (e.g., a poly T sequence, or a nucleic acid primer sequence complementary to a target nucleic acid sequence and/or for amplifying a target nucleic acid sequence, a random primer, and a primer sequence for messenger RNA).

In some cases, the nucleic acid barcode molecule further comprises a unique molecular identifier (UMI). In some cases, the nucleic acid barcode molecule comprises one or more functional sequences, for example, for attachment to a sequencing flow cell, such as, for example, a P5 sequence (or a portion thereof) for Illumina® sequencing. In some cases, the nucleic acid barcode molecule or derivative thereof (e.g., oligonucleotide or polynucleotide generated from the nucleic acid molecule) comprises another functional sequence, such as, for example, a P7 sequence (or a portion thereof) for attachment to a sequencing flow cell for Illumina sequencing. In some cases, the nucleic acid molecule can comprise an R1 primer sequence for Illumina sequencing. In some cases, the nucleic acid molecule comprises an R2 primer sequence for Illumina sequencing. In some cases, a functional sequence comprises a partial sequence, such as a partial barcode sequence, partial anchoring sequence, partial sequencing primer sequence (e.g., partial R1 sequence, partial R2 sequence, etc.), a partial sequence configured to attach to the flow cell of a sequencer (e.g., partial P5 sequence, partial P7 sequence, etc.), or a partial sequence of any other type of sequence described elsewhere herein. A partial sequence may contain a contiguous or continuous portion or segment, but not all, of a full sequence, for example. In some cases, a downstream procedure may extend the partial sequence, or derivative thereof, to achieve a full sequence of the partial sequence, or derivative thereof.

Examples of such nucleic acid molecules (e.g., oligonucleotides, polynucleotides, etc.) and uses thereof, as may be used with compositions, devices, methods and systems of the present disclosure, are provided in U.S. Patent Pub. Nos. 2014/0378345 and 2015/0376609, each of which is herein incorporated by reference in its entirety.

In some embodiments, a nucleic acid barcode molecule is coupled to a bead by a releasable linkage, such as, for example, a disulfide linker. The same bead may be coupled (e.g., via releasable linkage) to one or more other nucleic acid barcode molecules. In some instances, a nucleic acid barcode molecule comprises a barcode (e.g., a cell barcode sequence). As noted elsewhere herein, the structure of the barcode may comprise a number of sequence elements. In some embodiments, a nucleic acid barcode molecule comprises a functional sequence that may be used in subsequent processing. For example, the functional sequence comprises one or more of a sequencer specific flow cell attachment sequence (e.g., a P5 sequence for Illumina® sequencing systems) and a sequencing primer sequence (e.g., a R1 primer for Illumina® sequencing systems), or partial sequence(s) thereof. In some embodiments, a nucleic acid barcode molecule comprises a barcode sequence for use in barcoding the sample and identifying the cell of origin of the analytes (e.g., DNA, RNA, protein, etc.). In some cases, the barcode sequence (e.g., a cell barcode sequence) is bead-specific such that the barcode sequence is common to all nucleic acid barcode molecules coupled to the same bead. Alternatively, or in addition, the barcode sequence can be partition-specific such that the barcode sequence is common to all nucleic acid barcode molecules coupled to one or more beads that are partitioned into the same partition. In some instances, a nucleic acid barcode molecule comprises a sequence complementary to an analyte of interest, e.g., a priming sequence. For example, a nucleic acid barcode molecule (e.g., in FIG. 3, nucleic acid barcode molecule 310) comprises a poly-T sequence 313 complementary to a poly-A tail of an mRNA analyte, a targeted priming sequence, and/or a random priming sequence. In some instances, a nucleic acid barcode molecule comprises an anchoring sequence to ensure that the specific priming sequence hybridizes at the sequence end (e.g., of the mRNA). For example, the anchoring sequence can include a random short sequence of nucleotides, such as a 1-mer, 2-mer, 3-mer or longer sequence, which can ensure that a poly-T segment is more likely to hybridize at the sequence end of the poly-A tail of the mRNA.

In some instances, a nucleic acid barcode molecule comprises a unique molecular identifying sequence (e.g., unique molecular identifier (UMI)). In some cases, the unique molecular identifying sequence may comprise from about 5 to about 8 nucleotides. In some cases, the unique molecular identifying sequence comprises 5, 6, 7, or 8 nucleotides. Alternatively, the unique molecular identifying sequence may compress less than about 5 or more than about 8 nucleotides. In some instances, the unique molecular identifying sequence comprises 1, 2, 3, or 4 nucleotides. In some instances, the unique molecular identifying sequence comprises 9, 10, 11, 12, 13, 14, 15, or more nucleotides. The unique molecular identifying sequence may be a unique sequence that varies across individual nucleic acid barcode molecules (e.g., 310, 320, 350, etc.) coupled to a single bead (e.g., bead 330). In some cases, the unique molecular identifying sequence may be a random sequence (e.g., such as a random N-mer sequence). For example, the UMI may provide a unique identifier of the starting analyte (e.g., mRNA) molecule that was captured, in order to allow quantitation of the number of original expressed RNA molecules. As will be appreciated, although FIG. 3 shows three nucleic acid barcode molecules 310, 320, 350 coupled to the surface of the bead 330, an individual bead may be coupled to any number of individual nucleic acid barcode molecules, for example, from one to tens to hundreds of thousands, millions, or even a billion of individual nucleic acid barcode molecules. The respective barcodes for the individual nucleic acid barcode molecules can comprise both common sequence segments or relatively common sequence segments (e.g., 312) and variable or unique sequence segments between different individual nucleic acid barcode molecules coupled to the same bead.

In operation, a biological particle (e.g., cell, DNA, RNA, etc.) can be co-partitioned along with a barcode bearing bead 330. The nucleic acid barcode molecules 310, 320, 350 can be released from the bead 330 in the partition. By way of example, in the context of analyzing sample RNA, the poly-T segment (e.g., 313) of one of the released nucleic acid barcode molecules (e.g., 330) can hybridize to the poly-A tail of a mRNA molecule. Reverse transcription may result in a cDNA transcript of the mRNA, wherein the transcript includes each of the sequence segments 311 and 312 of the nucleic acid barcode molecule 310. Within any given partition, all of the cDNA transcripts of the individual mRNA molecules may include a common barcode sequence segment 312. However, the transcripts made from the different mRNA molecules within a given partition may vary at the unique molecular identifying sequence segment (e.g., UMI segment). Beneficially, even following any subsequent amplification of the contents of a given partition, the number of different UMIs can be indicative of the quantity of mRNA originating from a given partition, and thus from the biological particle (e.g., cell). As noted above, the transcripts can be amplified, cleaned up and sequenced to identify the sequence of the cDNA transcript of the mRNA, as well as to sequence the barcode segment and the UMI segment. While a poly-T primer sequence is described, other targeted or random priming sequences may also be used in priming the reverse transcription reaction. Likewise, although described as releasing the barcoded oligonucleotides into the partition, in some cases, the nucleic acid barcode molecules bound to the bead (e.g., gel bead) may be used to hybridize and capture the mRNA on the solid phase of the bead, for example, in order to facilitate the separation of the RNA from other cell contents. In such cases, further processing may be performed, in the partitions or outside the partitions (e.g., in bulk). For instance, the RNA molecules on the beads may be subjected to reverse transcription or other nucleic acid processing, additional adapter sequences may be added to the barcoded nucleic acid molecules, or other nucleic acid reactions (e.g., amplification, nucleic acid extension) may be performed. The beads or products thereof (e.g., barcoded nucleic acid molecules) may be collected from the partitions, and/or pooled together and subsequently subjected to clean up and further characterization (e.g., sequencing).

The operations described herein may be performed at any useful or convenient step. For instance, the beads comprising nucleic acid barcode molecules may be introduced into a partition (e.g., well or droplet) prior to, during, or following introduction of a sample into the partition. The nucleic acid molecules of a sample may be subjected to barcoding, which may occur on the bead (in cases where the nucleic acid molecules remain coupled to the bead) or following release of the nucleic acid barcode molecules into the partition. In cases where the nucleic acid molecules from the sample remain attached to the bead, the beads from various partitions may be collected, pooled, and subjected to further processing (e.g., reverse transcription, adapter attachment, amplification, clean up, sequencing). In other instances, the processing may occur in the partition. For example, conditions sufficient for barcoding, adapter attachment, reverse transcription, or other nucleic acid processing operations may be provided in the partition and performed prior to clean up and sequencing.

In some instances, a bead may comprise a capture sequence or binding sequence configured to bind to a corresponding capture sequence or binding sequence. In some instances, a bead may comprise a plurality of different capture sequences or binding sequences configured to bind to different respective corresponding capture sequences or binding sequences. For example, a bead may comprise a first subset of one or more capture sequences each configured to bind to a first corresponding capture sequence, a second subset of one or more capture sequences each configured to bind to a second corresponding capture sequence, a third subset of one or more capture sequences each configured to bind to a third corresponding capture sequence, and etc. A bead may comprise any number of different capture sequences. In some instances, a bead may comprise at least 2, at least 3, at least 4, at least 5, at least 6, at least 7, at least 8, at least 9, at least 10 or more different capture sequences or binding sequences configured to bind to different respective capture sequences or binding sequences, respectively. Alternatively, or in addition, a bead may comprise at most about 10, at most about 9, at most about 8, at most about 7, at most about 6, at most about 5, at most about 4, at most about 3, or at most about 2 different capture sequences or binding sequences configured to bind to different respective capture sequences or binding sequences. In some instances, the different capture sequences or binding sequences may be configured to facilitate analysis of a same type of analyte. In some instances, the different capture sequences or binding sequences may be configured to facilitate analysis of different types of analytes (with the same bead). The capture sequence may be designed to attach to a corresponding capture sequence. Beneficially, such corresponding capture sequence may be introduced to, or otherwise induced in, an biological particle (e.g., cell, cell bead, etc.) for performing different assays in various formats (e.g., barcoded antibodies comprising the corresponding capture sequence, barcoded MHC dextramers comprising the corresponding capture sequence, barcoded guide RNA molecules comprising the corresponding capture sequence, etc.), such that the corresponding capture sequence may later interact with the capture sequence associated with the bead. In some instances, a capture sequence coupled to a bead (or other support) may be configured to attach to a linker molecule, such as a splint molecule, wherein the linker molecule is configured to couple the bead (or other support) to other molecules through the linker molecule, such as to one or more analytes or one or more other linker molecules. In some embodiments, a capture sequence for interaction with an amplification product comprising a barcode sequence of the composite barcode is different from a capture sequence for interaction with an analyte (e.g., an mRNA or an oligonucleotide reporter associated with a labelling agent).

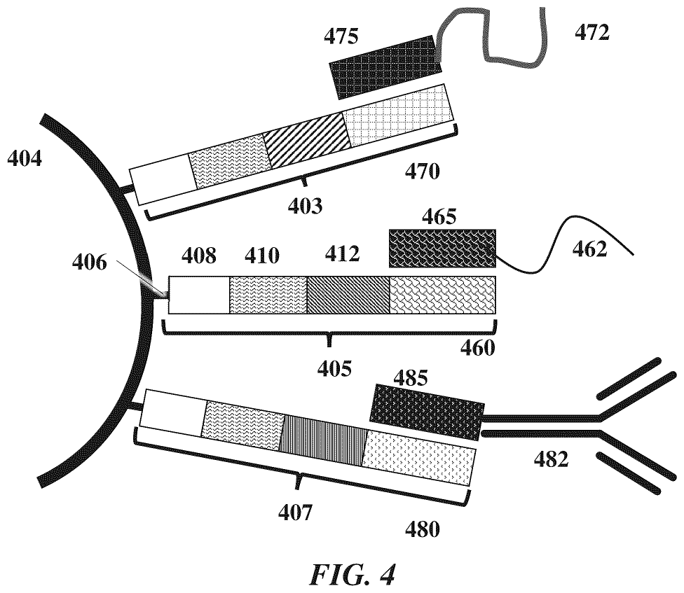

FIG. 4 illustrates another example of a barcode carrying bead. A nucleic acid barcode molecule 405, such as an oligonucleotide, can be coupled to a bead 404 by a releasable linkage 406, such as, for example, a disulfide linker. The nucleic acid barcode molecule 405 may comprise a first capture sequence 460. The same bead 404 may be coupled (e.g., via releasable linkage) to one or more other nucleic acid molecules 403, 407 comprising other capture sequences. The nucleic acid barcode molecule 405 may be or comprise a barcode. As noted elsewhere herein, the structure of the barcode may comprise a number of sequence elements, such as a functional sequence 408 (e.g., flow cell attachment sequence, sequencing primer sequence, etc.), a barcode sequence 410 (e.g., bead-specific sequence common to bead, partition-specific sequence common to partition, etc.), and a unique molecular identifier 412 (e.g., unique sequence within different molecules attached to the bead), or partial sequences thereof. The capture sequence 460 may be configured to attach to a corresponding capture sequence 465. In some instances, the corresponding capture sequence 465 may be coupled to another molecule that may be an analyte or an intermediary carrier. For example, as illustrated in FIG. 4, the corresponding capture sequence 465 is coupled to a cleaved portion of an amplification product comprising a barcode sequence of a composite barcode sequence 462. Another oligonucleotide molecule 407 attached to the bead 404 comprises a second capture sequence 480 which is configured to attach to a second corresponding capture sequence 485. As illustrated in FIG. 4, the second corresponding capture sequence 485 is coupled to an antibody 482. In some cases, the antibody 482 may have binding specificity to an analyte (e.g., surface protein). Alternatively, the antibody 482 may not have binding specificity. Another oligonucleotide molecule 403 attached to the bead 404 comprises a third capture sequence 470 which is configured to attach to a third corresponding capture sequence 475. As illustrated in FIG. 4, the third corresponding capture sequence 475 is coupled to a molecule 472. The molecule 472 may or may not be configured to target an analyte. The other oligonucleotide molecules 403, 407 may comprise the other sequences (e.g., functional sequence, barcode sequence, UMI, etc.) described with respect to oligonucleotide molecule 405. While a single oligonucleotide molecule comprising each capture sequence is illustrated in FIG. 4, it will be appreciated that, for each capture sequence, the bead may comprise a set of one or more oligonucleotide molecules each comprising the capture sequence. For example, the bead may comprise any number of sets of one or more different capture sequences. For example, the bead may comprise a capture sequence for coupling to a perturbation agent or a sequence associated with a perturbation agent. In some instances, the bead comprises a capture sequence for coupling to an immune molecule. In some instances, the bead comprises a capture sequence for coupling to an antigen receptor. In some instances, the bead comprises a capture sequence for coupling to a reporter oligonucleotide of a labelling agent. In some instances, the immune molecule is a V(D)J join comprising a V (variable) segment, a J (joint) segment, and optionally a D (diversity) segment between the V and J segments. Alternatively, or in addition, the bead 404 may comprise other capture sequences. Alternatively, or in addition, the bead 504 may comprise fewer types of capture sequences (e.g., two capture sequences). Alternatively, or in addition, the bead 404 may comprise oligonucleotide molecule(s) comprising a priming sequence, such as a specific priming sequence such as an mRNA specific priming sequence (e.g., poly-T sequence), a targeted priming sequence, and/or a random priming sequence, for example, to facilitate an assay for gene expression.

In operation, the barcoded oligonucleotides may be released (e.g., in a partition), as described elsewhere herein. Alternatively, the nucleic acid molecules bound to the bead (e.g., gel bead) may be used to hybridize and capture analytes (e.g., one or more types of analytes) on the solid phase of the bead.

A bead injected or otherwise introduced into a partition may comprise releasably, cleavably, or reversibly attached barcodes. A bead injected or otherwise introduced into a partition may comprise activatable barcodes. A bead injected or otherwise introduced into a partition may be degradable, disruptable, or dissolvable beads.

Barcodes can be releasably, cleavably or reversibly attached to the beads such that barcodes can be released or be releasable through cleavage of a linkage between the barcode molecule and the bead, or released through degradation of the underlying bead itself, allowing the barcodes to be accessed or be accessible by other reagents, or both. In non-limiting examples, cleavage may be achieved through reduction of di-sulfide bonds, use of restriction enzymes, photo-activated cleavage, or cleavage via other types of stimuli (e.g., chemical, thermal, pH, enzymatic, etc.) and/or reactions, such as described elsewhere herein. Releasable barcodes may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

As will be appreciated from the above disclosure, the degradation of a bead may refer to the disassociation of a bound or entrained species from a bead, both with and without structurally degrading the physical bead itself. For example, the degradation of the bead may involve cleavage of a cleavable linkage via one or more species and/or methods described elsewhere herein. In another example, entrained species may be released from beads through osmotic pressure differences due to, for example, changing chemical environments. See, e.g., PCT/US2014/044398, which is herein incorporated by reference in its entirety.

A degradable bead may be introduced into a partition, such as a droplet of an emulsion or a well, such that the bead degrades within the partition and any associated species (e.g., oligonucleotides) are released within the droplet when the appropriate stimulus is applied. The free species (e.g., oligonucleotides, nucleic acid molecules) may interact with other reagents contained in the partition. See, e.g., PCT/US2014/044398, which is herein incorporated by reference in its entirety.

As will be appreciated, barcodes that are releasably, cleavably or reversibly attached to the beads described herein include barcodes that are released or releasable through cleavage of a linkage between the barcode molecule and the bead, or that are released through degradation of the underlying bead itself, allowing the barcodes to be accessed or accessible by other reagents, or both.

In some cases, a species (e.g., oligonucleotide molecules comprising barcodes) that are attached to a solid support (e.g., a bead) may comprise a U-excising element that allows the species to release from the bead. In some cases, the U-excising element may comprise a single-stranded DNA (ssDNA) sequence that contains at least one uracil. The species may be attached to a solid support via the ssDNA sequence containing the at least one uracil. The species may be released by a combination of uracil-DNA glycosylase (e.g., to remove the uracil) and an endonuclease (e.g., to induce an ssDNA break). If the endonuclease generates a 5′ phosphate group from the cleavage, then additional enzyme treatment may be included in downstream processing to eliminate the phosphate group, e.g., prior to ligation of additional sequencing handle elements, e.g., Illumina full P5 sequence, partial P5 sequence, full R1 sequence, and/or partial R1 sequence.

The barcodes that are releasable as described herein may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

The nucleic acid barcode sequences can include from about 6 to about 20 or more nucleotides within the sequence of the nucleic acid molecules (e.g., oligonucleotides). The nucleic acid barcode sequences can include from about 6 to about 20, about 30, about 40, about 50, about 60, about 70, about 80, about 90, about 100 or more nucleotides. In some cases, the length of a barcode sequence may be about 6, about 7, about 8, about 9, about 10, about 11, about 12, about 13, about 14, about 15, about 16, about 17, about 18, about 19, about 20 nucleotides or longer. In some cases, the length of a barcode sequence may be at least about 6, at least about 7, at least about 8, at least about 9, at least about 10, at least about 11, at least about 12, at least about 13, at least about 14, at least about 15, at least about 16, at least about 17, at least about 18, at least about 19, at least about 20 nucleotides or longer. In some cases, the length of a barcode sequence may be at most about 6, at most about 7, at most about 8, at most about 9, at most about 10, at most about 11, at most about 12, at most about 13, at most about 14, at most about 15, at most about 16, at most about 17, at most about 18, at most about 19, at most about 20 nucleotides, or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be about 4, about 5, about 6, about 7, about 8, about 9, about 10, about 11, about 12, about 13, about 14, about 15, about 16 nucleotides or longer. In some cases, the barcode subsequence may be at least about 4, at least about 5, at least about 6, at least about 7, at least about 8, at least about 9, at least about 10, at least about 11, at least about 12, at least about 13, at least about 14, at least about 15, at least about 16 nucleotides, or longer. In some cases, the barcode subsequence may be at most about 4, at most about 5, at most about 6, at most about 7, at most about 8, at most about 9, at most about 10, at most about 11, at most about 12, at most about 13, at most about 14, at most about 15, at most about 16 nucleotides, or shorter.

The co-partitioned nucleic acid molecules can also comprise other functional sequences useful in the processing of the nucleic acids from the co-partitioned biological particles. These sequences include, e.g., targeted or random/universal amplification primer sequences for amplifying nucleic acids (e.g., mRNA, the genomic DNA) from the individual biological particles within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences (e.g., restriction sites, transposition sites). Other mechanisms of co-partitioning oligonucleotides may also be employed, including, e.g., coalescence of two or more droplets, where one droplet contains oligonucleotides, or microdispensing of oligonucleotides (e.g., attached to a bead) into partitions, e.g., droplets within microfluidic systems.

In an example, beads are provided that include large numbers of the above described nucleic acid barcode molecules releasably attached to the beads, where all or at least a subset of the nucleic acid barcode molecules attached to a particular bead will include a common nucleic acid barcode sequence, but where a large number of diverse barcode sequences are represented across the population of beads used. In some embodiments, hydrogel beads, e.g., comprising polyacrylamide polymer matrices, are used as a solid support and delivery vehicle for the nucleic acid barcode molecules into the partitions, as they are capable of carrying large numbers of nucleic acid barcode molecules, and may be configured to release those nucleic acid molecules upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. In some cases, the population of beads provides a diverse barcode sequence library that includes about 1,000 to about 10,000 different barcode sequences, about 5,000 to about 50,000 different barcode sequences, about 10,000 to about 100,000 different barcode sequences, about 50,000 to about 1,000,000 different barcode sequences, or about 100,000 to about 10,000,000 different barcode sequences.

Additionally, beads can be provided with large numbers of nucleic acid (e.g., oligonucleotide) molecules attached. In particular, the number of molecules of nucleic acid molecules including the barcode sequence on an individual bead can be at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules, or more. In some embodiments, the number of nucleic acid molecules including the barcode sequence on an individual bead is between about 1,000 to about 10,000 nucleic acid molecules, about 5,000 to about 50,000 nucleic acid molecules, about 10,000 to about 100,000 nucleic acid molecules, about 50,000 to about 1,000,000 nucleic acid molecules, about 100,000 to about 10,000,000 nucleic acid molecules, about 1,000,000 to about 1 billion nucleic acid molecules.

Nucleic acid molecules of a given bead can include identical (or common) barcode sequences, different barcode sequences, or a combination of both. Nucleic acid molecules of a given bead can include multiple sets of nucleic acid molecules. Nucleic acid molecules of a given set can include identical barcode sequences. The identical barcode sequences can be different from barcode sequences of nucleic acid molecules of another set.

Moreover, when the population of beads is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid barcode molecules, at least about 5,000 nucleic acid barcode molecules, at least about 10,000 nucleic acid barcode molecules, at least about 50,000 nucleic acid barcode molecules, at least about 100,000 nucleic acid barcode molecules, at least about 500,000 nucleic acids, at least about 1,000,000 nucleic acid barcode molecules, at least about 5,000,000 nucleic acid barcode molecules, at least about 10,000,000 nucleic acid barcode molecules, at least about 50,000,000 nucleic acid barcode molecules, at least about 100,000,000 nucleic acid barcode molecules, at least about 250,000,000 nucleic acid barcode molecules and in some cases at least about 1 billion nucleic acid barcode molecules.

In some cases, the resulting population of partitions provides a diverse barcode sequence library that includes about 1,000 to about 10,000 different barcode sequences, about 5,000 to about 50,000 different barcode sequences, about 10,000 to about 100,000 different barcode sequences, about 50,000 to about 1,000,000 different barcode sequences, or about 100,000 to about 10,000,000 different barcode sequences. Additionally, each partition of the population can include between about 1,000 to about 10,000 nucleic acid barcode molecules, about 5,000 to about 50,000 nucleic acid barcode molecules, about 10,000 to about 100,000 nucleic acid barcode molecules, about 50,000 to about 1,000,000 nucleic acid barcode molecules, about 100,000 to about 10,000,000 nucleic acid barcode molecules, about 1,000,000 to about 1 billion nucleic acid barcode molecules.

In some cases, it may be desirable to incorporate multiple different barcodes within a given partition, either attached to a single or multiple beads within the partition. For example, in some cases, a mixed, but known set of barcode sequences may provide greater assurance of identification in the subsequent processing, e.g., by providing a stronger address or attribution of the barcodes to a given partition, as a duplicate or independent confirmation of the output from a given partition.

The nucleic acid molecules (e.g., oligonucleotides) are releasable from the beads upon the application of a particular stimulus to the beads. In some cases, the stimulus may be a photo-stimulus, e.g., through cleavage of a photo-labile linkage that releases the nucleic acid molecules. In other cases, a thermal stimulus may be used, where elevation of the temperature of the beads environment will result in cleavage of a linkage or other release of the nucleic acid molecules from the beads. In still other cases, a chemical stimulus can be used that cleaves a linkage of the nucleic acid molecules to the beads, or otherwise results in release of the nucleic acid molecules from the beads. In one case, such compositions include the polyacrylamide matrices described above for encapsulation of biological particles and may be degraded for release of the attached nucleic acid molecules through exposure to a reducing agent, such as DTT.

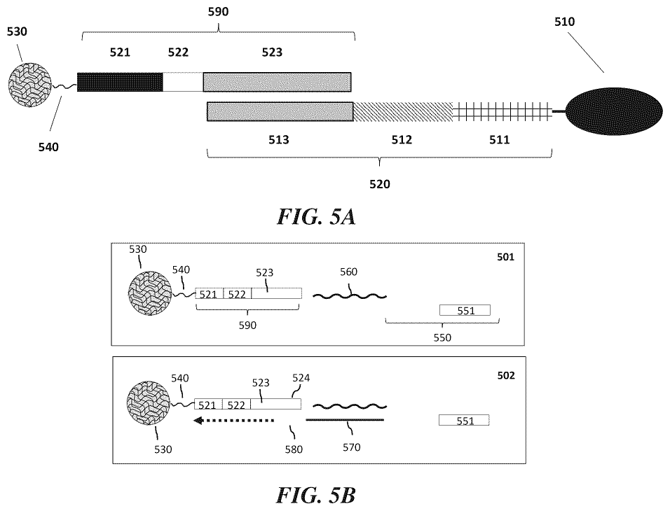

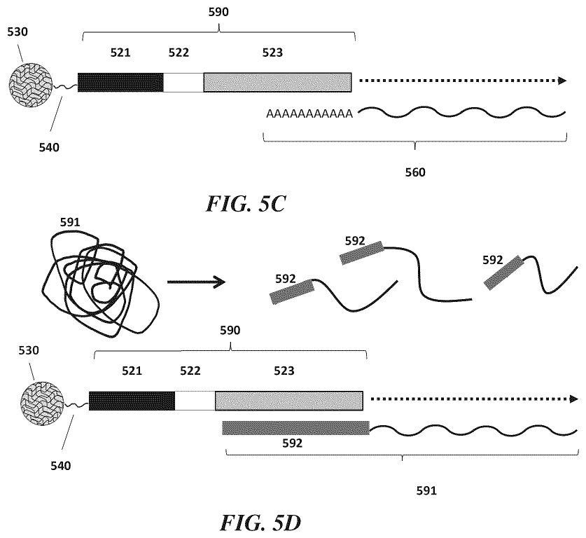

Referring to FIG. 5A, in an instance where cells are labelled with labelling agents, capture sequence 523 may be complementary to an adapter sequence of a reporter oligonucleotide. Cells may be contacted with one or more reporter oligonucleotide 520 conjugated labelling agents 510 (e.g., polypeptide, antibody, or others described elsewhere herein). In some cases, the cells may be further processed prior to barcoding. For example, such processing steps may include one or more washing and/or cell sorting steps. In some instances, a cell that is bound to labelling agent 510 which is conjugated to oligonucleotide 520 and support 530 (e.g., a bead, such as a gel bead) comprising nucleic acid barcode molecule 590 is partitioned into a partition amongst a plurality of partitions (e.g., a droplet of a droplet emulsion or a well of a microwell array). In some instances, the partition comprises at most a single cell bound to labelling agent 510. In some instances, reporter oligonucleotide 520 conjugated to labelling agent 510 (e.g., polypeptide, an antibody, pMHC molecule such as an MHC multimer, etc.) comprises a first adapter sequence 511 (e.g., a primer sequence), a barcode sequence 512 that identifies the labelling agent 510 (e.g., the polypeptide, antibody, or peptide of a pMHC molecule or complex), and an capture handle sequence 513. Capture handle sequence 513 may be configured to hybridize to a complementary sequence, such as a capture sequence 523 present on a nucleic acid barcode molecule 590. In some instances, oligonucleotide 520 comprises one or more additional functional sequences, such as those described elsewhere herein.

Barcoded nucleic acid molecules may be generated (e.g., via a nucleic acid reaction, such as nucleic acid extension or ligation) from the constructs described in FIGS. 5A-C. For example, capture handle sequence 513 may then be hybridized to complementary sequence, such as capture sequence 523 to generate (e.g., via a nucleic acid reaction, such as nucleic acid extension or ligation) a barcoded nucleic acid molecule comprising cell (e.g., partition specific) barcode sequence 522 (or a reverse complement thereof) and reporter barcode sequence 512 (or a reverse complement thereof). In some embodiments, the nucleic acid barcode molecule 590 (e.g., partition-specific barcode molecule) further includes a UMI (not shown). Barcoded nucleic acid molecules can then be optionally processed as described elsewhere herein, e.g., to amplify the molecules and/or append sequencing platform specific sequences to the fragments. See, e.g., U.S. Pat. Pub. 2018/0105808, which is hereby entirely incorporated by reference for all purposes. In some embodiments, barcoded nucleic acid molecules, or derivatives generated therefrom, are then sequenced on a suitable sequencing platform.

In some instances, analysis of multiple analytes (e.g., nucleic acids and one or more analytes using labelling agents described herein) are performed. For example, the workflow comprises a workflow as generally depicted in any of FIGS. 5A-5D, or a combination of workflows for an individual analyte, as described elsewhere herein. For example, by using a combination of the workflows as generally depicted in FIGS. 5A-5D, multiple analytes can be analyzed. In some embodiments, a nucleic acid molecule comprising a barcode sequence of the different barcode sequences of the composite barcode detected at a location in a biological sample before dissociating the cells or nuclei is analyzed. For example, a nucleic acid molecule comprising a barcode sequence of the different barcode sequences of the composite barcode is barcoded similarly as analytes.

In some instances, a barcoding reaction comprises a workflow as generally depicted in FIG. 5A. In some embodiments, the barcoding reaction is performed for appending a cell barcode or a complement thereof to the nucleic acid molecule comprising the composite barcode or a portion thereof and for analysis of an analyte (e.g. a nucleic acid, a polypeptide, a carbohydrate, a lipid, etc.) A nucleic acid barcode molecule 590 may be co-partitioned with the one or more analytes. In some instances, nucleic acid barcode molecule 590 is attached to a support 530 (e.g., a bead, such as a gel bead), such as those described elsewhere herein. For example, nucleic acid barcode molecule 590 is attached to support 530 via a releasable linkage 540 (e.g., comprising a labile bond), such as those described elsewhere herein. In some aspects, the nucleic acid barcode molecule 590 comprises a functional sequence 521 and optionally comprise other additional sequences, for example, a barcode sequence 522 (e.g., common cell barcode, partition-specific barcode, or other functional sequences described elsewhere herein), and/or a UMI sequence (not shown). In some aspects, the nucleic acid barcode molecule 590 comprises a capture sequence 523 that is complementary to another nucleic acid sequence, such that it hybridizes to a particular sequence, e.g., capture handle sequence 513.

For example, capture sequence 523 may comprise a poly-T sequence and may be used to hybridize to mRNA. Referring to FIG. 5C, in some embodiments, nucleic acid barcode molecule 590 comprises capture sequence 523 complementary to a sequence of RNA molecule 960 from a cell. In some instances, capture sequence 523 comprises a sequence specific for an RNA molecule. Capture sequence 523 may comprise a known or targeted sequence or a random sequence. In some instances, a nucleic acid extension reaction may be performed, thereby generating a barcoded nucleic acid product comprising capture sequence 523, the functional sequence 521, barcode sequence (e.g., cell barcode sequence) 522, any other functional sequence, and a sequence corresponding to the RNA molecule 560.

In some aspects, capture sequence 523 may comprise a capture sequence for hybridizing to a nucleic acid molecule comprising a sequence of the composite barcode. Referring to FIG. 5D, in some embodiments, nucleic acid barcode molecule 590 comprises capture sequence 523 complementary to a sequence of an amplification product (e.g., RCA product) 591 from a cell. In some instances, a barcode sequence of 591 is detected in situ at a location in the biological sample prior to the single cell barcoding reaction. In some cases, the barcode sequence in the amplification product 591 is part of the composite barcode generated in the cell. In some instances, capture sequence 523 comprises a common capture sequence for hybridizing to cleaved portions of RCA products. In some cases, multiple copies of the complement of the capture sequence 523 is generated in the amplification product 591. In some aspects, a plurality of amplification products are cleaved prior to the single cell barcoding reaction. In some aspects, a plurality of amplification products are cleaved after being detected in the cell at a location in the biological sample and prior to the single cell barcoding reaction. In some aspects, a plurality of amplification products are cleaved after dissociating cells or nuclei of the biological sample. In some aspects, a plurality of amplification products are cleaved prior to dissociating cells or nuclei of the biological sample. In some instances, a nucleic acid extension reaction may be performed, thereby generating a barcoded nucleic acid product comprising capture sequence 523, the functional sequence 521, barcode sequence (e.g., cell barcode sequence) 522, any other functional sequence, and a sequence corresponding to a barcode sequence of the amplification product 591.

In another example, capture sequence 523 may be complementary to an overhang sequence or an adapter sequence that has been appended to an analyte. For example, referring to FIG. 5B, panel 501, in some embodiments, primer 550 comprises a sequence complementary to a sequence of nucleic acid molecule 560 (such as an RNA encoding for a BCR sequence) from an biological particle. In some instances, primer 550 comprises one or more sequences 551 that are not complementary to RNA molecule 560. Sequence 551 may be a functional sequence as described elsewhere herein, for example, an adapter sequence, a sequencing primer sequence, or a sequence the facilitates coupling to a flow cell of a sequencer. In some instances, primer 550 comprises a poly-T sequence. In some instances, primer 550 comprises a sequence complementary to a target sequence in an RNA molecule. In some instances, primer 550 comprises a sequence complementary to a region of an immune molecule, such as the constant region of a TCR or BCR sequence. Primer 550 is hybridized to nucleic acid molecule 560 and complementary molecule 570 is generated (see Panel 502). For example, complementary molecule 570 may be cDNA generated in a reverse transcription reaction. In some instances, an additional sequence may be appended to complementary molecule 570. For example, the reverse transcriptase enzyme may be selected such that several non-templated bases 580 (e.g., a poly-C sequence) are appended to the cDNA. In another example, a terminal transferase may also be used to append the additional sequence. Nucleic acid barcode molecule 590 comprises a sequence 524 complementary to the non-templated bases, and the reverse transcriptase performs a template switching reaction onto nucleic acid barcode molecule 590 to generate a barcoded nucleic acid molecule comprising cell (e.g., partition specific) barcode sequence 522 (or a reverse complement thereof) and a sequence of complementary molecule 570 (or a portion thereof). In some instances, sequence 523 comprises a sequence complementary to a region of an immune molecule, such as the constant region of a TCR or BCR sequence. Sequence 523 is hybridized to nucleic acid molecule 560 and a complementary molecule 570 is generated. For example, complementary molecule 570 may be generated in a reverse transcription reaction generating a barcoded nucleic acid molecule comprising cell (e.g., partition specific) barcode sequence 522 (or a reverse complement thereof) and a sequence of complementary molecule 570 (or a portion thereof). Additional methods and compositions suitable for barcoding cDNA generated from mRNA transcripts including those encoding V(D)J regions of an immune cell receptor and/or barcoding methods and composition including a template switch oligonucleotide are described in International Patent Application WO2018/075693, U.S. Patent Pub. 2022/0389503, U.S. Pat. Pub. 2018/0105808, U.S. Pat. Pub. 2015/0376609, and U.S. Pat. No. 10,725,027 (originally published as U.S. Pat. Pub. 2019/0367969), each of which is herein incorporated by reference in its entirety.

In some embodiments, biological particles (e.g., cells, nuclei) from a plurality of samples (e.g., a plurality of subjects) can be pooled, sequenced, and demultiplexed by identifying mutational profiles associated with individual samples and mapping sequence data from single biological particles to their source based on their mutational profile. See, e.g., Xu J. et al., Genome Biology Vol. 20, 290 (2019); Huang Y. et al., Genome Biology Vol. 20, 273 (2019); and Heaton et al., Nature Methods volume 17, pages 615-620 (2020), each of which is herein incorporated by reference in its entirety.

Gene expression data can reflect the underlying genome and mutations and structural variants therein. As a result, the variation inherent in the captured and sequenced RNA molecules can be used to identify genotypes de novo or used to assign molecules to genotypes that were known a priori. In some embodiments, allelic variation that is present due to haplotypic states (including linkage disequilibrium of the human leucocyte antigen loci (HLA), immune receptor loci (BCR), and other highly polymorphic regions of the genome), can also be used for demultiplexing. Expressed B cell receptors can be used to infer germline alleles from unrelated individuals, which information may be used for demultiplexing.

### C. Templated Ligation of Probes

The methods described herein may comprise templated ligation. In some embodiments, a single cell barcoding reaction is performed and comprises a templated ligation reaction. For example, a templated ligation process comprises contacting a nucleic acid molecule (e.g., an RNA molecule or a nucleic acid molecule comprising a composite barcode or a portion thereof) with a probe molecule, such as a DNA probe, RNA probe, or a probe comprising both DNA and RNA. The probe molecule may interact with one or more other nucleic acid molecules, for example, those comprising a barcode sequence, to generate a probe-barcode complex. An extension reaction may be performed on at least a portion of the probe-barcode complex to generate a nucleic acid product that comprises the barcode sequence and is associated with a sequence of the nucleic acid molecule. Beneficially, the methods described herein may allow barcoding of the nucleic acid molecule without performing reverse transcription on the nucleic acid molecule. The methods herein may comprise ligation-mediated reactions.

In some embodiments, a first probe molecule is hybridized directly or indirectly to the analyte. In some aspects, the first probe molecule is hybridized to the analyte after dissociating the cells or nuclei. In some aspects, the first probe molecule is hybridized to the analyte before dissociating the cells or nuclei. In some aspects, an extension reaction is performed using at least a portion of the first probe molecule to generate a barcoded nucleic acid product of the plurality of barcoded nucleic acid products. In some embodiments, a second probe molecule is hybridized directly or indirectly to the analyte. In some aspects, the second probe molecule is hybridized to the analyte after dissociating the cells or nuclei. In some aspects, the second probe molecule is hybridized to the analyte before dissociating the cells or nuclei. In some aspects, an extension reaction is performed using at least a portion of the second probe molecule to generate a barcoded nucleic acid product of the plurality of barcoded nucleic acid products. In some embodiments, the first probe molecule and the second probe molecule are linked after hybridizing to the analyte. In some instances, the first probe molecule and the second probe molecule are linked by performing a ligation using the analyte or a splint molecule as template.

In some embodiments, a first probe molecule and a second probe molecule are linked after hybridizing to an analyte and an additional first probe molecule and an additional second probe molecule are hybridized to a fragmented portion of an amplification product in the same cell or nucleus. In some embodiments, the additional first probe molecule and additional second probe molecule hybridize to a barcode sequence of the composite barcode (e.g., generated using the probes described in Section II). In some aspects, hybridized probes are processed (e.g., partitioned) to append a cell barcode of the same sequence to the linked (e.g., by ligation) first and second probe molecules, and to the linked additional first and second probe molecules, thereby forming corresponding barcoded nucleic acid products. In some embodiments, the barcoded nucleic acid products are subject to sequencing and the common cell barcode is used to locate the analyte in the originating biological sample.

In some embodiments, a cell of the biological sample comprises multiple sequences of or associated with an analyte, wherein a first sequence of the analyte is contacted with a first probe molecule and a second probe molecule which are linked after hybridizing to the first sequence of the analyte, and a second sequence of the analyte is contacted with an additional first probe molecule and an additional second probe molecule which are linked after hybridizing to the second sequence of the analyte. In some embodiments, following hybridization, the cell contains multiple barcodes, wherein the multiple barcodes collectively comprise a composite barcode for identifying the cell. In some embodiments, multiple RCPs are generated within the cell. In some embodiments, the cell contains multiple RCPs, wherein each RCP comprises one or more barcode sequences.

In some embodiments, the first probe molecule hybridizes to a first sequence of the analyte and a second probe molecule hybridizes to a second sequence of the analyte and the first sequence and the second sequence of the analyte are separated by a gap of one or more nucleotides. In some instances, the gap between the first probe molecule and the second probe molecule hybridized to the first sequence and the second sequence is filled by performing an extension reaction.

A method may comprise contacting a nucleic acid molecule (e.g., an RNA molecule) with a first probe molecule, comprising a first sequence and a second sequence, under conditions sufficient for the first sequence to hybridize to a sequence of the nucleic acid molecule. A second probe molecule comprising a third sequence may hybridize to the second sequence of the first probe molecule. In some aspects, the first probe or the second probe molecule comprises a barcode sequence (e.g., as described herein). For example, the second probe molecule may be a nucleic acid molecule (e.g., as described herein). In some cases, a splint molecule may be used to link the first and second probe molecules. For example, a fourth sequence of the splint molecule may hybridize to the second sequence of the first probe molecule and a fifth sequence of the splint molecule may hybridize to the third sequence of the second probe molecule.

In another example, a first probe molecule with a first reactive moiety and a second probe molecule with a second reactive moiety may be used. A first sequence of the first probe molecule may hybridize to a first sequence of a nucleic acid molecule and a second sequence of the second probe molecule may hybridize to a second sequence of the nucleic acid molecule. The first sequence of the nucleic acid molecule and the second sequence of the nucleic acid molecule may be on the same nucleic acid strand. The first and second sequences of the nucleic acid molecule may be adjacent or may be separated by a gap of one or more nucleotides, which gap may optionally be filled (e.g., using a polymerase or one or more other relevant enzymes). The first reactive moiety of the first probe molecule and the second reactive moiety of the second probe molecule may be subjected to conditions sufficient for the first and second reactive moieties to react to provide a linking moiety. For example, a click chemistry reaction involving an alkyne moiety and an azide moiety may be used to provide a triazole linking moiety. In other examples, an iodide moiety may be chemically ligated to a phosphorothioate moiety to form a phosphorothioate bond, an acid may be ligated to an amine to form an amide bond, or a phosphate may be ligated to an amine to form a phosphoramidate bond. In some cases, the probes may be subjected to an enzymatic ligation reaction, using a ligase, e.g., SplintR ligases, T4 ligases, KOD ligases, PBCV1 enzymes, etc. to form a probe-linked nucleic acid molecule. Where the two probes are non-adjacent, gap regions between the probes may be filled prior to ligation. In some instances, ribonucleotides or deoxyribonucleotides are ligated between the first and second probes.

Prior to, in parallel, or subsequent to linking of the first and second probe molecules (e.g., via reaction between their respective reactive moieties), a third probe molecule (e.g., a nucleic acid barcode molecule) may be subjected to conditions sufficient to hybridize to a third sequence of the first probe molecule. The third probe molecule may comprise a barcode sequence. In some cases, a splint molecule may be used to link the first and third probe molecules. In some cases, the first and second probe molecules may be linked to one another such that a loop or “padlock” is formed after hybridization of the first sequence of the first probe molecule to the first sequence of the nucleic acid molecule and the second sequence of the second probe molecule to the second sequence of the nucleic acid molecule. A linkage between the first and second probe molecules may be generated after hybridization of the first and second probe molecules to the nucleic acid molecule, such as via reaction between two reactive moieties to form a linking moiety. Alternatively, the first and second probe molecules may be linked to one another before the first and second probe molecules hybridize to the nucleic acid molecule.

All or a portion of the templated ligation processes described herein may be performed within a partition (e.g., as described herein). Alternatively, one or more such processes may be performed within a bulk solution. For example, one or more probe molecules may be subjected to conditions sufficient to hybridize to a nucleic acid molecule (e.g., a nucleic acid molecule included in an biological particle such as a cell) within a bulk solution. The nucleic acid molecule may be partitioned within various reagents (e.g., as described herein) including a nucleic acid barcode molecule, such as a nucleic acid barcode molecule releasably coupled to a bead (e.g., as described herein). Within the partition, the nucleic acid barcode molecule may hybridize to a sequence of a probe molecule hybridized to the nucleic acid molecule, thereby generated a barcode-linked nucleic acid molecule. In some aspects, the ligation or linking of the first probe molecule and the second probe molecule occurs prior to partitioning, during partitioning, or after partitioning. In some aspects, the ligation or linking of the first probe molecule and the second probe molecule occurs while in the partition. Templated ligation processes may permit indirect barcoding of a nucleic acid molecule without the use of reverse transcription. Details of such processes and additional schemes are included in, for example, International Patent Application Publication Nos. WO2019/165318 and WO2021/041974, U.S. Patent Application Publication Nos. US20200239874, and U.S. Pat. No. 11,639,928, each of which is herein incorporated by reference in its entirety.

### D. Combinatorial Barcoding

In some embodiments, a single cell barcoding reaction is performed using a combinatorial approach. In some instances, one or more nucleic acid molecules (which may be comprised in a cell or cell bead) are partitioned (e.g., in a first set of partitions, e.g., wells or droplets) with one or more first nucleic acid barcode molecules (optionally coupled to a bead). The first nucleic acid barcode molecules or derivative thereof (e.g., complement, reverse complement) may then be attached to the one or more nucleic acid molecules, thereby generating first barcoded nucleic acid molecules, e.g., using the processes described herein. The first nucleic acid barcode molecules may be partitioned to the first set of partitions such that a nucleic acid barcode molecule, of the first nucleic acid barcode molecules, that is in a partition comprises a barcode sequence that is unique to the partition among the first set of partitions. Each partition may comprise a unique barcode sequence. For example, a set of first nucleic acid barcode molecules partitioned to a first partition in the first set of partitions may each comprise a common barcode sequence that is unique to the first partition among the first set of partitions, and a second set of first nucleic acid barcode molecules partitioned to a second partition in the first set of partitions may each comprise another common barcode sequence that is unique to the second partition among the first set of partitions. Such barcode sequence (unique to the partition) may be useful in determining the cell or partition from which the one or more nucleic acid molecules (or derivatives thereof) originated.

The first barcoded nucleic acid molecules from multiple partitions of the first set of partitions may be pooled and re-partitioned (e.g., in a second set of partitions, e.g., one or more wells or droplets) with one or more second nucleic acid barcode molecules. The second nucleic acid barcode molecules or derivative thereof may then be attached to the first barcoded nucleic acid molecules, thereby generating second barcoded nucleic acid molecules. As with the first nucleic acid barcode molecules during the first round of partitioning, the second nucleic acid barcode molecules may be partitioned to the second set of partitions such that a nucleic acid barcode molecule, of the second nucleic acid barcode molecules, that is in a partition comprises a barcode sequence that is unique to the partition among the second set of partitions. Such barcode sequence may also be useful in determining the cell or partition from which the one or more nucleic acid molecules or first barcoded nucleic acid molecules originated. The second barcoded nucleic acid molecules may thus comprise two barcode sequences (e.g., from the first nucleic acid barcode molecules and the second nucleic acid barcode molecules).

Additional barcode sequences may be attached to the second barcoded nucleic acid molecules by repeating the processes any number of times (e.g., in a split-and-pool approach), thereby combinatorically synthesizing unique barcode sequences to barcode the one or more nucleic acid molecules. For example, combinatorial barcoding may comprise at least 1, at least 2, at least 3, at least 4, at least 5, at least 6, at least 7, at least 8, at least 9, at least 10 or more operations of splitting (e.g., partitioning) and/or pooling (e.g., from the partitions). Additional examples of combinatorial barcoding may also be found in International Pat. Pub. WO2019/165318 and US Pat. Pub. US20200239874, each of which is herein incorporated by reference in its entirety.

Beneficially, the combinatorial barcode approach may be useful for generating greater barcode diversity and synthesizing unique barcode sequences on nucleic acid molecules derived from a cell or partition. For example, combinatorial barcoding comprising three operations, each with 100 partitions, may yield up to 106 unique barcode combinations. In some instances, the combinatorial barcode approach may be helpful in determining whether a partition contained only one cell or more than one cell. For instance, the sequences of the first nucleic acid barcode molecule and the second nucleic acid barcode molecule may be used to determine whether a partition comprised more than one cell. For instance, if two nucleic acid molecules comprise different first barcode sequences but the same second barcode sequences, it may be inferred that the second set of partitions comprised two or more cells.

In some instances, combinatorial barcoding may be achieved in the same compartment. For instance, a unique nucleic acid molecule comprising one or more nucleic acid bases may be attached to a nucleic acid molecule (e.g., a sample or target nucleic acid molecule) in successive operations within a partition (e.g., droplet or well) to generate a first barcoded nucleic acid molecule. A second unique nucleic acid molecule comprising one or more nucleic acid bases may be attached to the first barcoded nucleic acid molecule, thereby generating a second barcoded nucleic acid molecule. In some instances, all the reagents for barcoding and generating combinatorially barcoded molecules may be provided in a single reaction mixture, or the reagents may be provided sequentially.

In some instances, cell beads comprising nucleic acid molecules may be barcoded. Methods and systems for barcoding cell beads are further described in U.S. Pat. No. 12,104,200 (previously published as U.S. Pat. Pub. 2019/0330694), which is herein incorporated by reference in its entirety.

### E. Sample Compartmentalization

In an aspect, the systems and methods described herein provide for the compartmentalization, depositing, or partitioning of one or more particles (e.g., biological particles, macromolecular constituents of biological particles, beads, reagents, etc.) into discrete compartments or partitions (referred to interchangeably herein as partitions), where each partition maintains separation of its own contents from the contents of other partitions. The partition can be a droplet in an emulsion or a well. In some embodiments, a single cell barcoding reaction is performed by partitioning a dissociated cell or nucleus with a plurality of nucleic acid barcode molecules, wherein each nucleic acid barcode molecule of the plurality of nucleic acid barcode molecules comprises a capture sequence and the cell barcode. In some instances, the dissociated cell or nucleus is partitioned with a particle.

For example, a partition may include one or more particles. A partition may include one or more types of particles. For example, a partition of the present disclosure may comprise one or more biological particles and/or macromolecular constituents thereof. A partition may comprise one or more beads. A partition may comprise one or more gel beads. A partition may comprise one or more cell beads. A partition may include a single gel bead, a single cell bead, or both a single cell bead and single gel bead. A partition may include one or more reagents. Alternatively, a partition may be unoccupied. For example, a partition may not comprise a bead.

Unique identifiers, such as barcodes, may be injected into the droplets previous to, subsequent to, or concurrently with droplet generation, such as via a bead, as described elsewhere herein.

The methods and systems of the present disclosure may comprise methods and systems for generating one or more partitions such as droplets. The droplets may comprise a plurality of droplets in an emulsion. In some examples, the droplets may comprise droplets in a colloid. In some cases, the emulsion may comprise a microemulsion or a nanoemulsion. In some examples, the droplets may be generated with aid of a microfluidic device and/or by subjecting a mixture of immiscible phases to agitation (e.g., in a container). In some cases, a combination of the mentioned methods may be used for droplet and/or emulsion formation.

The partitions described herein may comprise small volumes, for example, less than about 10 microliters (μL), less than about 5 μL, less than about 1 μL, less than about 10 nanoliters (nL), less than about 5 nL, less than about 1 nL, less than about 900 picoliters (pL), less than about 800 pL, less than about 700 pL, less than about 600 pL, less than about 500 pL, less than about 400 pL, less than about 300 pL, less than about 200 pL, less than about 100 pL, less than about 50 pL, less than about 20 pL, less than about 10 pL, less than about 1 pL, less than about 500 nanoliters (nL), less than about 100 nL, less than about 50 nL, or less.

For example, in the case of droplet-based partitions, the droplets may have overall volumes that are less than about 1000 pL, less than about 900 pL, less than about 800 pL, less than about 700 pL, less than about 600 pL, less than about 500 pL, less than about 400 pL, less than about 300 pL, less than about 200 pL, less than about 100 pL, less than about 50 pL, less than about 20 pL, less than about 10 pL, less than about 1 pL, or less. Where co-partitioned with beads, it will be appreciated that the sample fluid volume, e.g., including co-partitioned biological particles and/or beads, within the partitions may be less than about 90% of the above described volumes, less than about 80%, less than about 70%, less than about 60%, less than about 50%, less than about 40%, less than about 30%, less than about 20%, or less than about 10% of the above described volumes.

As is described elsewhere herein, partitioning species may generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions may comprise both unoccupied partitions (e.g., empty partitions) and occupied partitions.

Droplets can be formed by creating an emulsion by mixing and/or agitating immiscible phases. Mixing or agitation may comprise various agitation techniques, such as vortexing, pipetting, tube flicking, or other agitation techniques. In some cases, mixing or agitation may be performed without using a microfluidic device. In some examples, the droplets may be formed by exposing a mixture to ultrasound or sonication. Systems and methods for droplet and/or emulsion generation by agitation are described in International Pat. Appl. No. PCT/US2020/017785 and U.S. Pat. Pub. US20220025438, each of which is entirely incorporated herein by reference for all purposes.

### F. Microfluidic Systems

Microfluidic devices or platforms comprising microfluidic channel networks (e.g., on a chip) can be utilized to generate partitions such as droplets and/or emulsions as described herein. Methods and systems for generating partitions such as droplets, methods of encapsulating biological particles in partitions, methods of increasing the throughput of droplet generation, and various geometries, architectures, and configurations of microfluidic devices and channels are described in U.S. Pat. Pubs. 2019/0367997 and 2019/0064173, each of which is herein incorporated by reference in its entirety.

In some examples, individual particles can be partitioned to discrete partitions by introducing a flowing stream of particles in an aqueous fluid into a flowing stream or reservoir of a non-aqueous fluid, such that droplets may be generated at the junction of the two streams/reservoir, such as at the junction of a microfluidic device provided elsewhere herein.

The methods of the present disclosure may comprise generating partitions and/or encapsulating particles, such as biological particles, in some cases, individual biological particles such as single cells. In some examples, reagents may be encapsulated and/or partitioned (e.g., co-partitioned with biological particles) in the partitions. Various mechanisms may be employed in the partitioning of individual particles. An example may comprise porous membranes through which aqueous mixtures of cells may be extruded into fluids (e.g., non-aqueous fluids).

The partitions can be flowable within fluid streams. The partitions may comprise, for example, micro-vesicles that have an outer barrier surrounding an inner fluid center or core. In some cases, the partitions may comprise a porous matrix that is capable of entraining and/or retaining materials within its matrix. The partitions can be droplets of a first phase within a second phase, wherein the first and second phases are immiscible. For example, the partitions can be droplets of aqueous fluid within a non-aqueous continuous phase (e.g., oil phase). In another example, the partitions can be droplets of a non-aqueous fluid within an aqueous phase. In some examples, the partitions may be provided in a water-in-oil emulsion or oil-in-water emulsion. A variety of different vessels are described in, for example, U.S. Pat. Pub. 2014/0155295, which is herein incorporated by reference in its entirety. Emulsion systems for creating stable droplets in non-aqueous or oil continuous phases are described in, for example, U.S. Pat. Pub. 2010/0105112, which is herein incorporated by reference in its entirety.

Fluid properties (e.g., fluid flow rates, fluid viscosities, etc.), particle properties (e.g., volume fraction, particle size, particle concentration, etc.), microfluidic architectures (e.g., channel geometry, etc.), and other parameters may be adjusted to control the occupancy of the resulting partitions (e.g., number of biological particles per partition, number of beads per partition, etc.). For example, partition occupancy can be controlled by providing the aqueous stream at a certain concentration and/or flow rate of particles. To generate single biological particle partitions, the relative flow rates of the immiscible fluids can be selected such that, on average, the partitions may contain less than one biological particle per partition to ensure that those partitions that are occupied are primarily singly occupied. In some cases, partitions among a plurality of partitions may contain at most one biological particle (e.g., bead, DNA, cell or cellular material). In some embodiments, the various parameters (e.g., fluid properties, particle properties, microfluidic architectures, etc.) may be selected or adjusted such that a majority of partitions are occupied, for example, allowing for only a small percentage of unoccupied partitions. The flows and channel architectures can be controlled as to ensure a given number of singly occupied partitions, less than a certain level of unoccupied partitions and/or less than a certain level of multiply occupied partitions.

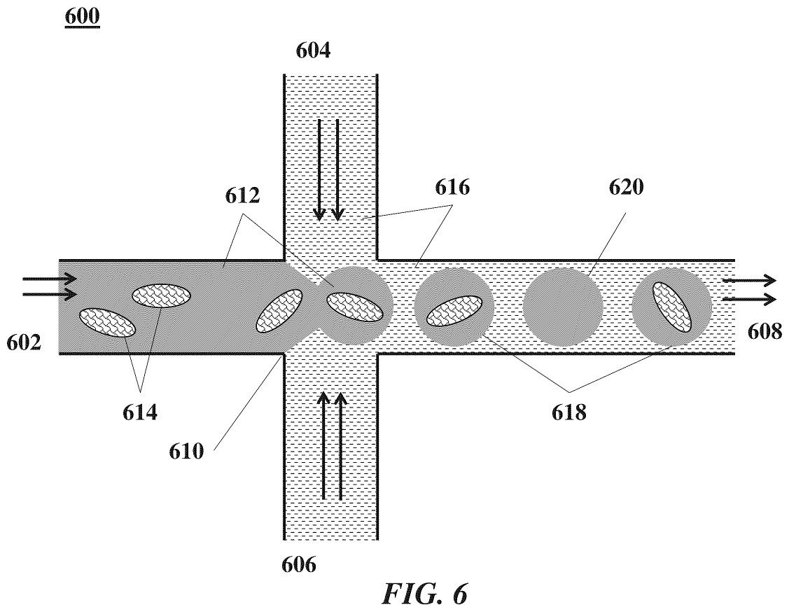

FIG. 6 shows an example of a microfluidic channel structure 600 for partitioning individual biological particles (e.g., dissociated cells or nuclei). The channel structure 600 can include channel segments 602, 604, 606 and 608 communicating at a channel junction 610. In operation, a first aqueous fluid 612 that includes suspended biological particles (or cells) 614 may be transported along channel segment 602 into junction 610, while a second fluid 616 that is immiscible with the aqueous fluid 612 is delivered to the junction 610 from each of channel segments 604 and 606 to create discrete droplets 618, 620 of the first aqueous fluid 612 flowing into channel segment 608, and flowing away from junction 610. The channel segment 608 may be fluidically coupled to an outlet reservoir where the discrete droplets can be stored and/or harvested. A discrete droplet generated may include an individual biological particle 614 (such as droplets 618). A discrete droplet generated may include more than one individual biological particle 614 (not shown in FIG. 6). A discrete droplet may contain no biological particle 614 (such as droplet 620). Each discrete partition may maintain separation of its own contents (e.g., individual biological particle 614) from the contents of other partitions.

The second fluid 616 can comprise an oil, such as a fluorinated oil, that includes a fluorosurfactant for stabilizing the resulting droplets, for example, inhibiting subsequent coalescence of the resulting droplets 618, 620. Examples of particularly useful partitioning fluids and fluorosurfactants are described, for example, in U.S. Patent Application Publication No. 2010/0105112, which is entirely incorporated herein by reference for all purposes.

As will be appreciated, the channel segments described herein may be coupled to any of a variety of different fluid sources or receiving components, including reservoirs, tubing, manifolds, or fluidic components of other systems. As will be appreciated, the microfluidic channel structure 600 may have other geometries. For example, a microfluidic channel structure can have more than one channel junction. For example, a microfluidic channel structure can have 2, 3, 4, or 5 channel segments each carrying particles (e.g., biological particles, cell beads, and/or gel beads) that meet at a channel junction. Fluid may be directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

The generated droplets may comprise two subsets of droplets: (1) occupied droplets 618, containing one or more biological particles 614, and (2) unoccupied droplets 620, not containing any biological particles 614. Occupied droplets 618 may comprise singly occupied droplets (having one biological particle) and multiply occupied droplets (having more than one biological particle). As described elsewhere herein, in some cases, the majority of occupied partitions can include no more than one biological particle per occupied partition and some of the generated partitions can be unoccupied (of any biological particle). In some cases, though, some of the occupied partitions may include more than one biological particle. In some cases, the partitioning process may be controlled such that fewer than about 25% of the occupied partitions contain more than one biological particle, and in many cases, fewer than about 20% of the occupied partitions have more than one biological particle, while in some cases, fewer than about 10% or even fewer than about 5% of the occupied partitions include more than one biological particle per partition.

In some cases, it may be desirable to minimize the creation of excessive numbers of empty partitions, such as to reduce costs and/or increase efficiency. While this minimization may be achieved by providing a sufficient number of biological particles (e.g., biological particles 614) at the partitioning junction 610, such as to ensure that at least one biological particle is encapsulated in a partition, the Poissonian distribution may expectedly increase the number of partitions that include multiple biological particles. As such, where singly occupied partitions are to be obtained, at most about 95%, at most about 90%, at most about 85%, at most about 80%, at most about 75%, at most about 70%, at most about 65%, at most about 60%, at most about 55%, at most about 50%, at most about 45%, at most about 40%, at most about 35%, at most about 30%, at most about 25%, at most about 20%, at most about 15%, at most about 10%, at most about 5%, or less of the generated partitions can be unoccupied.

In some cases, flows can be controlled so as to present a non-Poissonian distribution of single-occupied partitions while providing lower levels of unoccupied partitions (e.g., no more than about 50%, about 25%, or about 10% unoccupied). The above noted ranges of unoccupied partitions can be achieved while still providing any of the single occupancy rates described above.

As will be appreciated, the above-described occupancy rates are also applicable to partitions that include both biological particles and additional reagents, such as beads (e.g., gel beads) carrying nucleic acid barcode molecules (e.g., oligonucleotides).

In some examples, a partition of the plurality of partitions may comprise a single biological particle (e.g., a single cell or a single nucleus of a cell). In some examples, a partition of the plurality of partitions may comprise multiple biological particles. Such partitions may be referred to as multiply occupied partitions, and may comprise, for example, two, three, four or more cells and/or beads (e.g., beads) comprising nucleic acid barcode molecules within a single partition. Accordingly, as noted above, the flow characteristics of the biological particle and/or bead containing fluids and partitioning fluids may be controlled to provide for such multiply occupied partitions. In particular, the flow parameters may be controlled to provide a given occupancy rate at greater than about 50% of the partitions, greater than about 75%, and in some cases greater than about 80%, 90%, 95%, or higher.

Microfluidic systems for partitioning are further described in U.S. Pat. Pub. 2015/0376609, which is hereby incorporated by reference in its entirety.

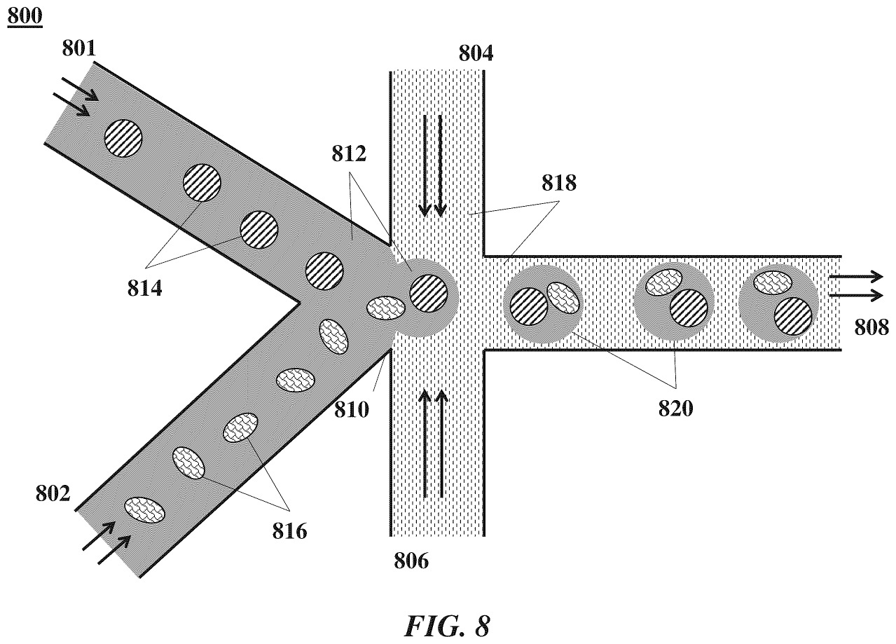

FIG. 8 shows an example of a microfluidic channel structure 800 for delivering barcode carrying beads to droplets. The channel structure 800 can include channel segments 801, 802, 804, 806 and 808 communicating at a channel junction 810. In operation, the channel segment 801 may transport an aqueous fluid 812 that includes a plurality of beads 814 (e.g., with nucleic acid molecules, e.g., nucleic acid barcode molecules or barcoded oligonucleotides, molecular tags) along the channel segment 801 into junction 810. The plurality of beads 814 may be sourced from a suspension of beads. For example, the channel segment 801 may be connected to a reservoir comprising an aqueous suspension of beads 814. The channel segment 802 may transport the aqueous fluid 812 that includes a plurality of biological particles 816 along the channel segment 802 into junction 810. The plurality of biological particles 816 may be sourced from a suspension of biological particles. For example, the channel segment 802 may be connected to a reservoir comprising an aqueous suspension of biological particles 816. In some instances, the aqueous fluid 812 in either the first channel segment 801 or the second channel segment 802, or in both segments, can include one or more reagents, as further described below. A second fluid 818 that is immiscible with the aqueous fluid 812 (e.g., oil) can be delivered to the junction 810 from each of channel segments 804 and 806. Upon meeting of the aqueous fluid 812 from each of channel segments 801 and 802 and the second fluid 818 from each of channel segments 804 and 806 at the channel junction 810, the aqueous fluid 812 can be partitioned as discrete droplets 820 in the second fluid 818 and flow away from the junction 810 along channel segment 808. The channel segment 808 may deliver the discrete droplets to an outlet reservoir fluidly coupled to the channel segment 308, where they may be harvested. As an alternative, the channel segments 801 and 802 may meet at another junction upstream of the junction 810. At such junction, beads and biological particles may form a mixture that is directed along another channel to the junction 810 to yield droplets 820. The mixture may provide the beads and biological particles in an alternating fashion, such that, for example, a droplet comprises a single bead and a single biological particle.

In some aspects, provided are systems and methods for controlled partitioning. Droplet size may be controlled by adjusting certain geometric features in channel architecture (e.g., microfluidics channel architecture). For example, an expansion angle, width, and/or length of a channel may be adjusted to control droplet size.

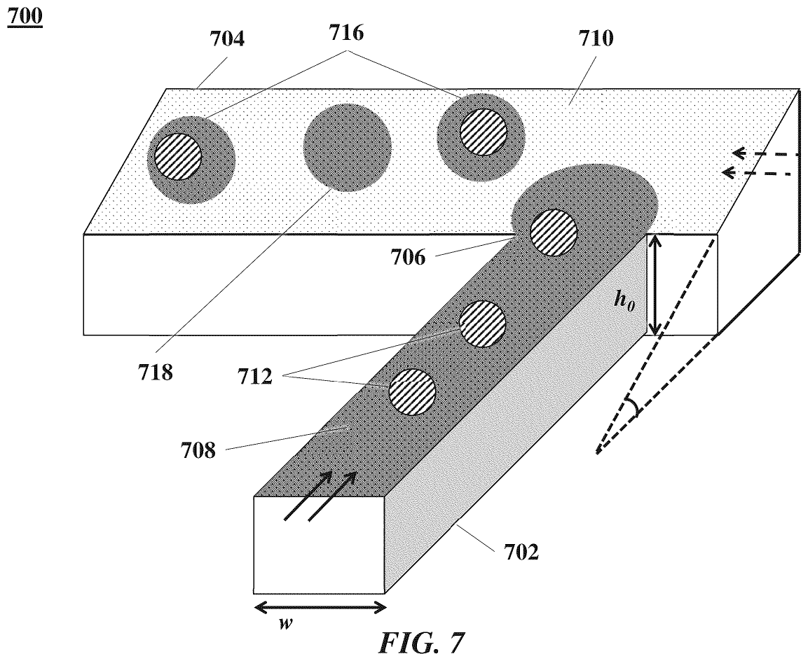

FIG. 7 shows an example of a microfluidic channel structure for the controlled partitioning of beads into discrete droplets. A channel structure 700 can include a channel segment 702 communicating at a channel junction 706 (or intersection) with a reservoir 704. The reservoir 704 can be a chamber. Any reference to “reservoir,” as used herein, can also refer to a “chamber.” In operation, an aqueous fluid 708 that includes suspended beads 712 may be transported along the channel segment 702 into the junction 706 to meet a second fluid 710 that is immiscible with the aqueous fluid 708 in the reservoir 704 to create droplets 716, 718 of the aqueous fluid 708 flowing into the reservoir 704. At the junction 706 where the aqueous fluid 708 and the second fluid 710 meet, droplets can form based on factors such as the hydrodynamic forces at the junction 706, flow rates of the two fluids 708, 710, fluid properties, and certain geometric parameters (e.g., w, ho, a, etc.) of the channel structure 700. A plurality of droplets can be collected in the reservoir 704 by continuously injecting the aqueous fluid 708 from the channel segment 702 through the junction 706.

In some instances, the aqueous fluid 708 can have a substantially uniform concentration or frequency of beads 712. The beads 712 can be introduced into the channel segment 702 from a separate channel (not shown in FIG. 7). The frequency of beads 712 in the channel segment 702 may be controlled by controlling the frequency in which the beads 712 are introduced into the channel segment 702 and/or the relative flow rates of the fluids in the channel segment 702 and the separate channel. In some instances, the beads is introduced into the channel segment 702 from a plurality of different channels, and the frequency controlled accordingly.

In some instances, the aqueous fluid 708 in the channel segment 702 can comprise biological particles. In some instances, the aqueous fluid 708 can have a substantially uniform concentration or frequency of biological particles. As with the beads, the biological particles can be introduced into the channel segment 702 from a separate channel. The frequency or concentration of the biological particles in the aqueous fluid 708 in the channel segment 702 may be controlled by controlling the frequency in which the biological particles are introduced into the channel segment 702 and/or the relative flow rates of the fluids in the channel segment 702 and the separate channel. In some instances, the biological particles can be introduced into the channel segment 702 from a plurality of different channels, and the frequency controlled accordingly. In some instances, a first separate channel can introduce beads and a second separate channel can introduce biological particles into the channel segment 702. The first separate channel introducing the beads may be upstream or downstream of the second separate channel introducing the biological particles.

The second fluid 710 can comprise an oil, such as a fluorinated oil, that includes a fluorosurfactant for stabilizing the resulting droplets, for example, inhibiting subsequent coalescence of the resulting droplets.

In some instances, the second fluid 710 may not be subjected to and/or directed to any flow in or out of the reservoir 704. For example, the second fluid 710 may be substantially stationary in the reservoir 704. In some instances, the second fluid 710 may be subjected to flow within the reservoir 704, but not in or out of the reservoir 704, such as via application of pressure to the reservoir 704 and/or as affected by the incoming flow of the aqueous fluid 708 at the junction 706. Alternatively, the second fluid 710 may be subjected and/or directed to flow in or out of the reservoir 704. For example, the reservoir 704 can be a channel directing the second fluid 710 from upstream to downstream, transporting the generated droplets.

Systems and methods for controlled partitioning are described further in International Pat. Appl. PCT/US2018/047551 and U.S. Patent Application Publication No. US2020/0290048, each of which is herein incorporated by reference in its entirety.

### G. Cell Beads

In another aspect, in addition to or as an alternative to droplet-based partitioning, biological particles (e.g., cells) may be comprised within (e.g., encapsulated within) a particulate material to form a “cell bead”. Methods and compositions drawn to cell beads and the like are described further in International Patent Application No. PCT/US2018/016019 and U.S. Pat. Pub. US2018/0216162, each of which is herein incorporated by reference in its entirety.

A cell bead can contain a biological particle (e.g., a cell) or macromolecular constituents (e.g., RNA, DNA, proteins, etc.) of a biological particle. A cell bead may include a single cell or multiple cells, or a derivative of the single cell or multiple cells. For example after lysing and washing the cells, inhibitory components from cell lysates can be washed away and the macromolecular constituents can be bound as cell beads. Systems and methods disclosed herein can be applicable to both cell beads (and/or droplets or other partitions) containing biological particles and cell beads (and/or droplets or other partitions) containing macromolecular constituents of biological particles. Cell beads may be or include a cell, cell derivative, cellular material and/or material derived from the cell in, within, or encased in a matrix, such as a polymeric matrix. In some cases, a cell bead may comprise a live cell. In some instances, the live cell may be capable of being cultured when enclosed in a gel or polymer matrix, or of being cultured when comprising a gel or polymer matrix. In some instances, the polymer or gel may be diffusively permeable to certain components and diffusively impermeable to other components (e.g., macromolecular constituents).

Cell beads can provide certain potential advantages of being more storable and more portable than droplet-based partitioned biological particles. Furthermore, in some cases, it may be desirable to allow biological particles to incubate for a select period of time before analysis, such as in order to characterize changes in such biological particles over time, either in the presence or absence of different stimuli (or reagents).

Suitable polymers or gels may include one or more of disulfide cross-linked polyacrylamide, agarose, alginate, polyvinyl alcohol, polyethylene glycol (PEG)-diacrylate, PEG-acrylate, PEG-thiol, PEG-azide, PEG-alkyne, other acrylates, chitosan, hyaluronic acid, collagen, fibrin, gelatin, or elastin. The polymer or gel may comprise any other polymer or gel.

Encapsulation of biological particles may be performed by a variety of processes. Such processes may combine an aqueous fluid containing the biological particles with a polymeric precursor material that may be capable of being formed into a gel or other solid or semi-solid matrix upon application of a particular stimulus to the polymer precursor. The conditions sufficient to polymerize or gel the precursors may comprise any conditions sufficient to polymerize or gel the precursors. Such stimuli can include, for example, thermal stimuli (e.g., either heating or cooling), photo-stimuli (e.g., through photo-curing), chemical stimuli (e.g., through crosslinking, polymerization initiation of the precursor (e.g., through added initiators)), electromagnetic radiation, mechanical stimuli, or any combination thereof.

In some cases, air knife droplet or aerosol generators may be used to dispense droplets of precursor fluids into gelling solutions in order to form cell beads that include individual biological particles or small groups of biological particles. Likewise, membrane-based encapsulation systems may be used to generate cell beads comprising encapsulated biological particles as described herein. Microfluidic systems of the present disclosure, such as that shown in FIG. 6, may be readily used in encapsulating biological particles (e.g., cells) as described herein. Exemplary methods for encapsulating biological particles (e.g., cells) are also further described in U.S. Patent Application Pub. No. US 2015/0376609 and International Patent Application No. PCT/US2018/016019, which are hereby incorporated by reference in their entirety. In particular, and with reference to FIG. 6, the aqueous fluid 612 comprising (i) the biological particles 614 and (ii) the polymer precursor material (not shown) is flowed into channel junction 610, where it is partitioned into droplets 618, 620 through the flow of non-aqueous fluid 616. In the case of encapsulation methods, non-aqueous fluid 616 may also include an initiator (not shown) to cause polymerization and/or crosslinking of the polymer precursor to form the bead that includes the entrained biological particles. Examples of polymer precursor/initiator pairs include those described in U.S. Pat. Pub. 2014/0378345, which is herein incorporated by reference in its entirety.

In some cases, encapsulated biological particles can be selectively releasable from the cell bead, such as through passage of time or upon application of a particular stimulus, that degrades the bead sufficiently to allow the biological particles (e.g., cell), or its other contents to be released from the bead, such as into a partition (e.g., droplet). Exemplary stimuli suitable for degradation of the bead are described in U.S. Pat. Pub. 2014/0378345, which is herein incorporated by reference in its entirety.

The polymer or gel may be diffusively permeable to chemical or biochemical reagents. The polymer or gel may be diffusively impermeable to macromolecular constituents of the biological particle. In this manner, the polymer or gel may act to allow the biological particle to be subjected to chemical or biochemical operations while spatially confining the macromolecular constituents to a region of the droplet defined by the polymer or gel.

The polymer or gel may be functionalized to bind to targeted analytes, such as nucleic acids, proteins, carbohydrates, lipids or other analytes. The polymer or gel may be polymerized or gelled via a passive mechanism. The polymer or gel may be stable in alkaline conditions or at elevated temperature. The polymer or gel may have mechanical properties similar to the mechanical properties of the bead. For instance, the polymer or gel may be of a similar size to the bead. The polymer or gel may have a mechanical strength (e.g. tensile strength) similar to that of the bead. The polymer or gel may be of a lower density than an oil. The polymer or gel may be of a density that is roughly similar to that of a buffer. The polymer or gel may have a tunable pore size. The pore size may be chosen to, for instance, retain denatured nucleic acids. The pore size may be chosen to maintain diffusive permeability to exogenous chemicals such as sodium hydroxide (NaOH) and/or endogenous chemicals such as inhibitors. The polymer or gel may be biocompatible. The polymer or gel may maintain or enhance cell viability. The polymer or gel may be biochemically compatible. The polymer or gel may be polymerized and/or depolymerized thermally, chemically, enzymatically, and/or optically.

The encapsulation of biological particles may constitute the partitioning of the biological particles into which other reagents are co-partitioned. Alternatively, or in addition, encapsulated biological particles may be readily deposited into other partitions (e.g., droplets) as described above.

### H. Beads

Nucleic acid barcode molecules may be delivered to a partition (e.g., a droplet or well) via a solid support or carrier (e.g., a bead). In some cases, nucleic acid barcode molecules are initially associated with the solid support and then released from the solid support upon application of a stimulus, which allows the nucleic acid barcode molecules to dissociate or to be released from the solid support. In specific examples, nucleic acid barcode molecules are initially associated with the solid support (e.g., bead) and then released from the solid support upon application of a biological stimulus, a chemical stimulus, a thermal stimulus, an electrical stimulus, a magnetic stimulus, and/or a photo stimulus.

The solid support may be a bead. A solid support, e.g., a bead, may be porous, non-porous, hollow, solid, semi-solid, and/or a combination thereof. Beads may be solid, semi-solid, semi-fluidic, fluidic, and/or a combination thereof. In some instances, a solid support, e.g., a bead, may be at least partially dissolvable, disruptable, and/or degradable. In some cases, a solid support, e.g., a bead, may not be degradable. In some cases, the solid support, e.g., a bead, may be a gel bead. A gel bead may be a hydrogel bead. A gel bead may be formed from molecular precursors, such as a polymeric or monomeric species. A semi-solid support, e.g., a bead, may be a liposomal bead. Solid supports, e.g., beads, may comprise metals including iron oxide, gold, and silver. In some cases, the solid support, e.g., the bead, may be a silica bead. In some cases, the solid support, e.g., a bead, can be rigid. In other cases, the solid support, e.g., a bead, may be flexible and/or compressible.

A partition may comprise one or more unique identifiers, such as barcodes. Barcodes may be previously, subsequently or concurrently delivered to the partitions that hold the compartmentalized or partitioned biological particle. For example, barcodes may be injected into droplets or deposited in microwells previous to, subsequent to, or concurrently with droplet generation or providing of reagents in the microwells, respectively. The delivery of the barcodes to a particular partition allows for the later attribution of the characteristics of the individual biological particle to the particular partition. Barcodes may be delivered, for example on a nucleic acid molecule (e.g., via a nucleic acid barcode molecule), to a partition via any suitable mechanism. Nucleic acid barcode molecules can be delivered to a partition via a bead. Beads are described in further detail below.

In some cases, nucleic acid barcode molecules can be initially associated with the bead and then released from the bead. Release of the nucleic acid barcode molecules can be passive (e.g., by diffusion out of the bead). In addition, or alternatively, release from the bead can be upon application of a stimulus which allows the nucleic acid barcode molecules to dissociate or to be released from the bead. Such stimulus may disrupt the bead, an interaction that couples the nucleic acid barcode molecules to or within the bead, or both. Such stimulus can include, for example, a thermal stimulus, photo-stimulus, chemical stimulus (e.g., change in pH or use of a reducing agent(s)), a mechanical stimulus, a radiation stimulus; a biological stimulus (e.g., enzyme), or any combination thereof.

Methods and systems for partitioning barcode carrying beads into droplets are provided herein, and in U.S. Pat. Pub. 2019/0367997, U.S. Pat. Pub. 2019/0064173, and International Patent Application No. PCT/US20/17785, each of which is herein incorporated by reference in its entirety.

A bead may be porous, non-porous, solid, semi-solid, semi-fluidic, fluidic, and/or a combination thereof. In some instances, a bead may be dissolvable, disruptable, and/or degradable. Degradable beads, as well as methods for degrading beads, are described in International Patent Application Publication No. PCT/US2014/044398, which is herein incorporated by reference in its entirety. In some cases, any combination of stimuli, e.g., stimuli described in PCT/US2014/044398 and US Patent Application Pub. No. 2015/0376609, each of which is herein incorporated by reference in its entirety, may trigger degradation of a bead. For example, a change in pH may enable a chemical agent (e.g., DTT) to become an effective reducing agent. In other examples, a reducing agent (e.g., DTT) may be used to degrade the bead.

In some cases, a bead may not be degradable. In some cases, the bead may be a gel bead. A gel bead may be a hydrogel bead. A gel bead may be formed from molecular precursors, such as a polymeric or monomeric species. A semi-solid bead may be a liposomal bead. Solid beads may comprise metals including iron oxide, gold, and silver. In some cases, the bead may be a silica bead. In some cases, the bead can be rigid. In other cases, the bead may be flexible and/or compressible.

A bead may be of any suitable shape. Examples of bead shapes include, but are not limited to, spherical, non-spherical, oval, oblong, amorphous, circular, cylindrical, and variations thereof.

Beads may be of uniform size or heterogeneous size. In some cases, the diameter of a bead may be at least about 10 nanometers (nm), at least about 100 nm, at least about 500 nm, at least about 1 micrometer (μm), at least about 5 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 250 μm, at least about 500 μm, at least about 1 mm, or greater. In some cases, a bead may have a diameter of less than about 10 nm, less than about 100 nm, less than about 500 nm, less than about 1 μm, less than about 5 μm, less than about 10 μm, less than about 20 μm, less than about 30 μm, less than about 40 μm, less than about 50 μm, less than about 60 μm, less than about 70 μm, less than about 80 μm, less than about 90 μm, less than about 100 μm, less than about 250 μm, less than about 500 μm, less than about 1 mm, or less. In some cases, a bead may have a diameter in the range of about 40-75 μm, about 30-75 μm, about 20-75 μm, about 40-85 μm, about 40-95 μm, about 20-100 μm, about 10-100 μm, about 1-100 μm, about 20-250 μm, or about 20-500 μm.

In certain aspects, beads can be provided as a population or plurality of beads having a relatively monodisperse size distribution. Where it may be desirable to provide relatively consistent amounts of reagents within partitions, maintaining relatively consistent bead characteristics, such as size, can contribute to the overall consistency. In particular, the beads described herein may have size distributions that have a coefficient of variation in their cross-sectional dimensions of less than 50%, less than 40%, less than 30%, less than 20%, and in some cases less than 15%, less than 10%, less than 5%, or less.

A bead may comprise natural and/or synthetic materials. For example, a bead can comprise a natural polymer, a synthetic polymer or both natural and synthetic polymers. See, e.g., PCT/US2014/044398, which is hereby incorporated by reference in its entirety. Beads may also be formed from materials other than polymers, including lipids, micelles, ceramics, glass-ceramics, material composites, metals, other inorganic materials, and others.

In some cases, the bead may comprise covalent or ionic bonds between polymeric precursors (e.g., monomers, oligomers, linear polymers), nucleic acid barcode molecules (e.g., oligonucleotides), primers, and other entities. In some cases, the covalent bonds can be carbon-carbon bonds, thioether bonds, or carbon-heteroatom bonds.

In some cases, a plurality of nucleic acid barcode molecules may be attached to a bead. The nucleic acid barcode molecules may be attached directly or indirectly to the bead. In some cases, the nucleic acid barcode molecules may be covalently linked to the bead. In some cases, the nucleic acid barcode molecules are covalently linked to the bead via a linker. In some cases, the linker is a degradable linker. In some cases, the linker comprises a labile bond configured to release the nucleic acid barcode molecule of said plurality of nucleic acid barcode molecules. In some cases, the labile bond comprises a disulfide linkage.

Activation or disruption of disulfide linkages within a bead can be controlled such that only a small number of disulfide linkages are activated or disrupted. Methods of controlling activation of disulfide linkages within a bead are described in International Patent Application No. PCT/US2014/044398, which is herein incorporated by reference in its entirety.

In some cases, a bead may comprise an acrydite moiety, which in certain aspects may be used to attach one or more nucleic acid barcode molecules (e.g., barcode sequence, nucleic acid barcode molecule, barcoded oligonucleotide, primer, or other oligonucleotide) to the bead. Acrydite moieties, as well as their uses in attaching nucleic acid molecules to beads, are described in PCT/US2014/044398, which is herein incorporated by reference in its entirety.

For example, precursors (e.g., monomers, cross-linkers) that are polymerized to form a bead may comprise acrydite moieties, such that when a bead is generated, the bead also comprises acrydite moieties. The acrydite moieties can be attached to a nucleic acid molecule, e.g., a nucleic acid barcode molecule described herein.

In some cases, precursors comprising a functional group that is reactive or capable of being activated such that it becomes reactive can be polymerized with other precursors to generate gel beads comprising the activated or activatable functional group. The functional group may then be used to attach additional species (e.g., disulfide linkers, primers, other oligonucleotides, etc.) to the gel beads. Exemplary precursors comprising functional groups are described in PCT/US2014/044398, which is hereby incorporated by reference in its entirety.

Other non-limiting examples of labile bonds that may be coupled to a precursor or bead are described in PCT/US2014/044398, which is herein incorporated by reference in its entirety. A bond may be cleavable via other nucleic acid molecule targeting enzymes, such as restriction enzymes (e.g., restriction endonucleases), as described further below.

In some cases, a plurality of nucleic acid barcode molecules may be attached to a bead via non-covalent bonds. For example, the plurality of nucleic acid barcode molecules may be associated with a bead via an ionic interaction, electrostatic interactions, metallic bond, hydrogen bonding, van der Waals interactions, etc. In some cases, the non-covalent bond may be degraded upon application of a stimulus, e.g., a thermal, photo, magnetic, electrical, chemical stimulus (e.g., change in pH, ion concentration, etc.).

Species may be encapsulated in beads during bead generation (e.g., during polymerization of precursors). Such species may or may not participate in polymerization. See, e.g., PCT/US2014/044398, which is herein incorporated by reference in its entirety. Such species may include, for example, nucleic acid molecules (e.g., oligonucleotides), reagents for a nucleic acid amplification reaction (e.g., primers, polymerases, dNTPs, co-factors (e.g., ionic co-factors), buffers) including those described herein, reagents for enzymatic reactions (e.g., enzymes, co-factors, substrates, buffers), reagents for nucleic acid modification reactions such as polymerization, ligation, or digestion, and/or reagents for template preparation (e.g., tagmentation) for one or more sequencing platforms (e.g., Nextera® for Illumina®). Such species may include one or more enzymes described herein, including without limitation, polymerase, reverse transcriptase, restriction enzymes (e.g., endonuclease), transposase, ligase, proteinase K, DNAse, etc. Such species may include one or more reagents described elsewhere herein (e.g., lysis agents, inhibitors, inactivating agents, chelating agents, stimulus). Alternatively, or in addition, species may be partitioned in a partition (e.g., droplet) during or subsequent to partition formation. Such species may include, without limitation, the abovementioned species that may also be encapsulated in a bead.

In some cases, beads can be non-covalently loaded with and/or coupled to one or more reagents. The beads can be non-covalently loaded by, for instance, subjecting the beads to conditions sufficient to swell the beads, allowing sufficient time for the reagents to diffuse into the interiors of the beads, and subjecting the beads to conditions sufficient to de-swell the beads. The swelling of the beads may be accomplished, for instance, by placing the beads in a thermodynamically favorable solvent, subjecting the beads to a higher or lower temperature, subjecting the beads to a higher or lower ion concentration, and/or subjecting the beads to an electric field. The swelling of the beads may be accomplished by various swelling methods. The de-swelling of the beads may be accomplished, for instance, by transferring the beads in a thermodynamically unfavorable solvent, subjecting the beads to lower or high temperatures, subjecting the beads to a lower or higher ion concentration, and/or removing an electric field. The de-swelling of the beads may be accomplished by various de-swelling methods. Transferring the beads may cause pores in the bead to shrink. The shrinking may then hinder reagents within the beads from diffusing out of the interiors of the beads. The hindrance may be due to steric interactions between the reagents and the interiors of the beads. The transfer may be accomplished microfluidically. For instance, the transfer may be achieved by moving the beads from one co-flowing solvent stream to a different co-flowing solvent stream. The swellability and/or pore size of the beads may be adjusted by changing the polymer composition of the bead.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing oligonucleotide bearing beads.

### I. Reagents

In accordance with certain aspects, biological particles (e.g., a dissociated cell or nucleus) may be partitioned along with lysis reagents in order to release the contents of the biological particles within the partition. In such cases, the lysis agents can be contacted with the biological particle suspension concurrently with, or immediately prior to, the introduction of the biological particles into the partitioning junction/droplet generation zone such as through an additional channel or channels upstream of the channel junction. In accordance with other aspects, additionally or alternatively, biological particles may be partitioned along with other reagents, as will be described further below.

The methods and systems of the present disclosure may comprise microfluidic devices and methods of use thereof, which may be used for co-partitioning biological particles with reagents. Such systems and methods are described in U.S. Patent Publication No. US/20190367997, which is herein incorporated by reference in its entirety for all purposes.

Beneficially, when lysis reagents and biological particles are co-partitioned, the lysis reagents can facilitate the release of the contents of the biological particles within the partition. The contents released in a partition may remain discrete from the contents of other partitions.

As will be appreciated, the channel segments of the microfluidic devices described elsewhere herein may be coupled to any of a variety of different fluid sources or receiving components, including reservoirs, tubing, manifolds, or fluidic components of other systems. As will be appreciated, the microfluidic channel structures may have various geometries and/or configurations. For example, a microfluidic channel structure can have more than two channel junctions. For example, a microfluidic channel structure can have any of 2, 3, 4, 5 channel segments or more each carrying the same or different types of beads, reagents, and/or biological particles that meet at a channel junction. Fluid flow in each channel segment may be controlled to control the partitioning of the different elements into droplets. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, MO), as well as other commercially available lysis enzymes. Other lysis agents may additionally or alternatively be co-partitioned with the biological particles to cause the release of the biological particle's contents into the partitions. For example, in some cases, surfactant-based lysis solutions may be used to lyse cells. In some cases, lysis solutions may include non-ionic surfactants such as, for example, TritonX-100 and Tween 20. In some cases, lysis solutions may include ionic surfactants such as, for example, sarcosyl and sodium dodecyl sulfate (SDS). Electroporation, thermal, acoustic or mechanical cellular disruption may also be used in certain cases, e.g., non-emulsion-based partitioning such as encapsulation of biological particles that may be in addition to or in place of droplet partitioning, where any pore size of the encapsulate is sufficiently small to retain nucleic acid fragments of a given size, following cellular disruption.

Alternatively, or in addition to, the lysis agents co-partitioned with the biological particles described above, other reagents can also be co-partitioned with the biological particles, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated biological particles (e.g., a cell or a nucleus in a polymer matrix), the biological particles may be exposed to an appropriate stimulus to release the biological particles or their contents from a co-partitioned bead. For example, in some cases, a chemical stimulus may be co-partitioned along with an encapsulated biological particle to allow for the degradation of the bead and release of the cell or its contents into the larger partition. In some cases, this stimulus may be the same as the stimulus described elsewhere herein for release of nucleic acid molecules (e.g., oligonucleotides) from their respective bead. In alternative examples, this may be a different and non-overlapping stimulus, in order to allow an encapsulated biological particle to be released into a partition at a different time from the release of nucleic acid molecules into the same partition. For a description of methods, compositions, and systems for encapsulating cells (also referred to as a “cell bead”), see, e.g., U.S. Pat. No. 10,428,326 and U.S. Pat. Pub. 20190100632, each of which is herein incorporated by reference in its entirety.

Additional reagents may also be co-partitioned with the biological particle, such as endonucleases to fragment an biological particle's DNA, DNA polymerase enzymes and dNTPs used to amplify the biological particle's nucleic acid fragments and to attach the barcode molecular tags to the amplified fragments. Other enzymes may be co-partitioned, including without limitation, polymerase, transposase, ligase, proteinase K, DNAse, restriction enzymes, etc. Additional reagents may also include reverse transcriptase enzymes, including enzymes with terminal transferase activity, primers and oligonucleotides, and switch oligonucleotides (also referred to herein as “switch oligos” or “template switching oligonucleotides”) which can be used for template switching.

In some cases, template switching can be used to increase the length of a cDNA. In some cases, template switching can be used to append a predefined nucleic acid sequence to the cDNA. Template switching is further described in International Patent Application No. PCT/US2017/068320 and U.S. Pat. No. 10,011,872, each of which is herein incorporated by reference in its entirety. Template switching oligonucleotides may comprise a hybridization region and a template region. Template switching oligonucleotides are further described in PCT/US2017/068320 and U.S. Pat. No. 10,011,872, each of which is herein incorporated by reference in its entirety.

Any of the reagents described in this disclosure may be encapsulated in, or otherwise coupled to, a droplet, or bead, with any chemicals, particles, and elements suitable for sample processing reactions involving biomolecules, such as, but not limited to, nucleic acid molecules and proteins. For example, a bead or droplet used in a sample preparation reaction for DNA sequencing may comprise one or more of the following reagents: enzymes, restriction enzymes (e.g., multiple cutters), ligase, polymerase, fluorophores, oligonucleotide barcodes, adapters, buffers, nucleotides (e.g., dNTPs, ddNTPs) and the like.

Additional examples of reagents include, but are not limited to buffers, acidic solution, basic solution, temperature-sensitive enzymes, pH-sensitive enzymes, light-sensitive enzymes, metals, metal ions, magnesium chloride, sodium chloride, manganese, aqueous buffer, mild buffer, ionic buffer, inhibitor, enzyme, protein, polynucleotide, antibodies, saccharides, lipid, oil, salt, ion, detergents, ionic detergents, non-ionic detergents, and oligonucleotides.

Once the contents of the cells are released into their respective partitions, the macromolecular components (e.g., macromolecular constituents of biological particles, such as RNA, DNA, or proteins) contained therein may be further processed within the partitions. In accordance with the methods and systems described herein, the macromolecular component contents of individual biological particles can be provided with unique identifiers such that, upon characterization of those macromolecular components they may be attributed as having been derived from the same biological particle or particles. The ability to attribute characteristics to individual biological particles or groups of biological particles is provided by the assignment of unique identifiers specifically to an individual biological particle or groups of biological particles. Unique identifiers, e.g., in the form of nucleic acid barcodes can be assigned or associated with individual biological particles or populations of biological particles, in order to tag or label the biological particle's macromolecular components (and as a result, its characteristics) with the unique identifiers. These unique identifiers can then be used to attribute the biological particle's components and characteristics to an individual biological particle or group of biological particles. In some aspects, this is performed by co-partitioning the individual biological particle or groups of biological particles with the unique identifiers.

In some cases, additional beads can be used to deliver additional reagents to a partition. In such cases, it may be advantageous to introduce different beads into a common channel or droplet generation junction, from different bead sources (e.g., containing different associated reagents) through different channel inlets into such common channel or droplet generation junction. In such cases, the flow and frequency of the different beads into the channel or junction may be controlled to provide for a certain ratio of beads from each source, while ensuring a given pairing or combination of such beads into a partition with a given number of biological particles (e.g., one biological particle and one bead per partition).

In some embodiments, following the generation of barcoded nucleic acid molecules according to methods disclosed herein, subsequent operations that can be performed can include generation of amplification products, purification (e.g., via solid phase reversible immobilization (SPRI)), further processing (e.g., shearing, ligation of functional sequences, and subsequent amplification (e.g., via PCR)). These operations may occur in bulk (e.g., outside the partition). In the case where a partition is a droplet in an emulsion, the emulsion can be broken, and the contents of the droplet pooled for additional operations.

### J. Wells

As described herein, one or more processes may be performed in a partition, which may be a well. The well may be a well of a plurality of wells of a substrate, such as a microwell of a microwell array or plate, or the well may be a microwell or microchamber of a device (e.g., microfluidic device) comprising a substrate. The well may be a well of a well array or plate, or the well may be a well or chamber of a device (e.g., fluidic device). In some embodiments, a well of a fluidic device is fluidically connected to another well of the fluidic device. Accordingly, the wells or microwells may assume an “open” configuration, in which the wells or microwells are exposed to the environment (e.g., contain an open surface) and are accessible on one planar face of the substrate, or the wells or microwells may assume a “closed” or “sealed” configuration, in which the microwells are not accessible on a planar face of the substrate. In some instances, the wells or microwells may be configured to toggle between “open” and “closed” configurations. For instance, an “open” microwell or set of microwells may be “closed” or “sealed” using a membrane (e.g., semi-permeable membrane), an oil (e.g., fluorinated oil to cover an aqueous solution), or a lid, as described elsewhere herein.

The well may have a volume of less than 1 milliliter (mL). For instance, the well may be configured to hold a volume of at most 1000 microliters (μL), at most 100 μL, at most 10 μL, at most 1 μL, at most 100 nanoliters (nL), at most 10 nL, at most 1 nL, at most 100 picoliters (pL), at most 10 (pL), or less. The well may be configured to hold a volume of about 1000 μL, about 100 μL, about 10 μL, about 1 μL, about 100 nL, about 10 nL, about 1 nL, about 100 pL, about 10 pL, etc. The well may be configured to hold a volume of at least 10 pL, at least 100 pL, at least 1 nL, at least 10 nL, at least 100 nL, at least 1 μL, at least 10 μL, at least 100 μL, at least 1000 μL, or more. The well may be configured to hold a volume in a range of volumes listed herein, for example, from about 5 nL to about 20 nL, from about 1 nL to about 100 nL, from about 500 pL to about 100 μL, etc. The well may be of a plurality of wells that have varying volumes and may be configured to hold a volume appropriate to accommodate any of the partition volumes described herein.

In some instances, a microwell array or plate comprises a single variety of microwells. In some instances, a microwell array or plate comprises a variety of microwells. For instance, the microwell array or plate may comprise one or more types of microwells within a single microwell array or plate. The types of microwells may have different dimensions (e.g., length, width, diameter, depth, cross-sectional area, etc.), shapes (e.g., circular, triangular, square, rectangular, pentagonal, hexagonal, heptagonal, octagonal, nonagonal, decagonal, etc.), aspect ratios, or other physical characteristics. The microwell array or plate may comprise any number of different types of microwells. For example, the microwell array or plate may comprise any of 1, 2, 3, 4, 5, 6, 7, 8, 9, 10, 20, 30, 40, 50, 60, 70, 80, 90, 100, 200, 300, 400, 500, 600, 700, 800, 900, 1000 or more different types of microwells. A well may have any dimension (e.g., length, width, diameter, depth, cross-sectional area, volume, etc.), shape (e.g., circular, triangular, square, rectangular, pentagonal, hexagonal, heptagonal, octagonal, nonagonal, decagonal, other polygonal, etc.), aspect ratios, or other physical characteristics described herein with respect to any well.

In certain instances, the microwell array or plate comprises different types of microwells that are located adjacent to one another within the array or plate. For instance, a microwell with one set of dimensions may be located adjacent to and in contact with another microwell with a different set of dimensions. Similarly, microwells of different geometries may be placed adjacent to or in contact with one another. The adjacent microwells may be configured to hold different articles; for example, one microwell may be used to contain a cell, cell bead, or other sample (e.g., cellular components, nucleic acid molecules, etc.) while the adjacent microwell may be used to contain a droplet, bead, or other reagent. In some cases, the adjacent microwells may be configured to merge the contents held within, e.g., upon application of a stimulus, or spontaneously, upon contact of the articles in each microwell.

As is described elsewhere herein, a plurality of partitions may be used in the systems, compositions, and methods described herein. For example, any suitable number of partitions (e.g., wells or droplets) can be generated or otherwise provided. For example, in the case when wells are used, at least about 1,000 wells, at least about 5,000 wells, at least about 10,000 wells, at least about 50,000 wells, at least about 100,000 wells, at least about 500,000 wells, at least about 1,000,000 wells, at least about 5,000,000 wells at least about 10,000,000 wells, at least about 50,000,000 wells, at least about 100,000,000 wells, at least about 500,000,000 wells, at least about 1,000,000,000 wells, or more wells can be generated or otherwise provided. Moreover, the plurality of wells may comprise both unoccupied wells (e.g., empty wells) and occupied wells.

A well may comprise any of the reagents described herein, or combinations thereof. These reagents may include, for example, barcode molecules, enzymes, adapters, and combinations thereof. The reagents may be physically separated from a sample (e.g., a cell, cell bead, or cellular components, e.g., proteins, nucleic acid molecules, etc.) that is placed in the well. This physical separation may be accomplished by containing the reagents within, or coupling to, a bead that is placed within a well. The physical separation may also be accomplished by dispensing the reagents in the well and overlaying the reagents with a layer that is, for example, dissolvable, meltable, or permeable prior to introducing the polynucleotide sample into the well. This layer may be, for example, an oil, wax, membrane (e.g., semi-permeable membrane), or the like. The well may be sealed at any point, for example, after addition of the bead, after addition of the reagents, or after addition of either of these components. The sealing of the well may be useful for a variety of purposes, including preventing escape of beads or loaded reagents from the well, permitting select delivery of certain reagents (e.g., via the use of a semi-permeable membrane), for storage of the well prior to or following further processing, etc.

Once sealed, the well may be subjected to conditions for further processing of a cell (or cells) in the well. For instance, reagents in the well may allow further processing of the cell, e.g., cell lysis, as further described herein. Alternatively, the well (or wells such as those of a well-based array) comprising the cell (or cells) may be subjected to freeze-thaw cycling to process the cell (or cells), e.g., cell lysis. The well containing the cell may be subjected to freezing temperatures (e.g., 0° C., below 0° C., −5° C., −10° C., −15° C., −20° C., −25° C., −30° C., −35° C., −40° C., −45° C., −50° C., −55° C., −60° C., −65° C., −70° C., −80° C., or −85° C.). Freezing may be performed in a suitable manner, e.g., sub-zero freezer or a dry ice/ethanol bath. Following an initial freezing, the well (or wells) comprising the cell (or cells) may be subjected to freeze-thaw cycles to lyse the cell (or cells). In one embodiment, the initially frozen well (or wells) are thawed to a temperature above freezing (e.g., 4° C. or above, 8° C. or above, 12° C. or above, 16° C. or above, 20° C. or above, room temperature, or 25° C. or above). In another embodiment, the freezing is performed for less than 10 minutes (e.g., 5 minutes or 7 minutes) followed by thawing at room temperature for less than 10 minutes (e.g., 5 minutes or 7 minutes). This freeze-thaw cycle may be repeated a number of times, e.g., 2, 3, 4 or more times, to obtain lysis of the cell (or cells) in the well (or wells). In one embodiment, the freezing, thawing and/or freeze/thaw cycling is performed in the absence of a lysis buffer. Additional disclosure related to freeze-thaw cycling is provided in WO2019165181A1 and in U.S. Pat. Pub. US2021/0087549, each of which is incorporated herein by reference in its entirety.

A well may comprise free reagents and/or reagents encapsulated in, or otherwise coupled to or associated with, beads, beads, or droplets.

The wells may be provided as a part of a kit. For example, a kit may comprise instructions for use, a microwell array or device, and reagents (e.g., beads). The kit may comprise any useful reagents for performing the processes described herein, e.g., nucleic acid reactions, barcoding of nucleic acid molecules, sample processing (e.g., for cell lysis, fixation, and/or permeabilization).

In some cases, a well comprises a bead, or droplet that comprises a set of reagents that has a similar attribute (e.g., a set of enzymes, a set of minerals, a set of oligonucleotides, a mixture of different barcode molecules, a mixture of identical barcode molecules). In other cases, a bead or droplet comprises a heterogeneous mixture of reagents. In some cases, the heterogeneous mixture of reagents can comprise all components necessary to perform a reaction. In some cases, such mixture can comprise all components necessary to perform a reaction, except for 1, 2, 3, 4, 5, or more components necessary to perform a reaction. In some cases, such additional components are contained within, or otherwise coupled to, a different droplet or bead, or within a solution within a partition (e.g., microwell) of the system.

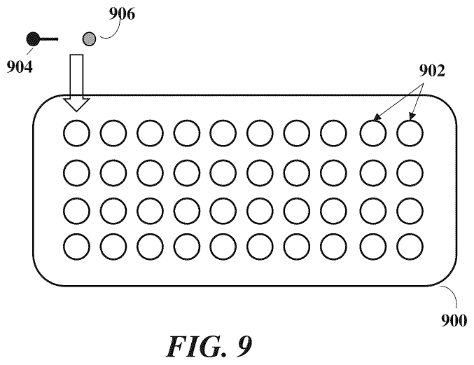

FIG. 9 schematically illustrates an example of a microwell array. The array can be contained within a substrate 900. The substrate 900 comprises a plurality of wells 902. The wells 902 may be of any size or shape, and the spacing between the wells, the number of wells per substrate, as well as the density of the wells on the substrate 900 can be modified, depending on the particular application. In one such example application, a sample molecule 906, which may comprise a cell or cellular components (e.g., nucleic acid molecules) is co-partitioned with a bead 904, which may comprise a nucleic acid barcode molecule coupled thereto. The wells 902 may be loaded using gravity or other loading technique (e.g., centrifugation, liquid handler, acoustic loading, optoelectronic, etc.). In some instances, at least one of the wells 902 contains a single sample molecule 906 (e.g., cell) and a single bead 904.

Reagents may be loaded into a well either sequentially or concurrently. In some cases, reagents are introduced to the device either before or after a particular operation. In some cases, reagents (which may be provided, in certain instances, in droplets, or beads) are introduced sequentially such that different reactions or operations occur at different steps. The reagents (or droplets, or beads) may also be loaded at operations interspersed with a reaction or operation step. For example, beads (or droplets) comprising reagents for fragmenting polynucleotides (e.g., restriction enzymes) and/or other enzymes (e.g., transposases, ligases, polymerases, etc.) may be loaded into the well or plurality of wells, followed by loading of droplets, or beads comprising reagents for attaching nucleic acid barcode molecules to a sample nucleic acid molecule. Reagents may be provided concurrently or sequentially with a sample, e.g., a cell or cellular components (e.g., organelles, proteins, nucleic acid molecules, carbohydrates, lipids, etc.). Accordingly, use of wells may be useful in performing multi-step operations or reactions.

As described elsewhere herein, the nucleic acid barcode molecules and other reagents may be contained within a bead, or droplet. These beads, or droplets may be loaded into a partition (e.g., a microwell) before, after, or concurrently with the loading of a cell, such that each cell is contacted with a different bead, or droplet. This technique may be used to attach a unique nucleic acid barcode molecule to nucleic acid molecules obtained from each cell. Alternatively, or in addition to, the sample nucleic acid molecules may be attached to a support. For instance, the partition (e.g., microwell) may comprise a bead which has coupled thereto a plurality of nucleic acid barcode molecules. The sample nucleic acid molecules, or derivatives thereof, may couple or attach to the nucleic acid barcode molecules on the support. The resulting barcoded nucleic acid molecules may then be removed from the partition, and in some instances, pooled and sequenced. In such cases, the nucleic acid barcode sequences may be used to trace the origin of the sample nucleic acid molecule. For example, polynucleotides with identical barcodes may be determined to originate from the same cell or partition, while polynucleotides with different barcodes may be determined to originate from different cells or partitions.

The samples or reagents may be loaded in the wells or microwells using a variety of approaches. The samples (e.g., a cell, cell bead, or cellular component) or reagents (as described herein) may be loaded into the well or microwell using an external force, e.g., gravitational force, electrical force, magnetic force, or using mechanisms to drive the sample or reagents into the well, e.g., via pressure-driven flow, centrifugation, optoelectronics, acoustic loading, electrokinetic pumping, vacuum, capillary flow, etc. In certain cases, a fluid handling system may be used to load the samples or reagents into the well. The loading of the samples or reagents may follow a Poissonian distribution or a non-Poissonian distribution, e.g., super Poisson or sub-Poisson. The geometry, spacing between wells, density, and size of the microwells may be modified to accommodate a useful sample or reagent distribution; for instance, the size and spacing of the microwells may be adjusted such that the sample or reagents may be distributed in a super-Poissonian fashion.

In one particular non-limiting example, the microwell array or plate comprises pairs of microwells, in which each pair of microwells is configured to hold a droplet (e.g., comprising a single cell) and a single bead (such as those described herein, which may, in some instances, also be encapsulated in a droplet). The droplet and the bead (or droplet containing the bead) may be loaded simultaneously or sequentially, and the droplet and the bead may be merged, e.g., upon contact of the droplet and the bead, or upon application of a stimulus (e.g., external force, agitation, heat, light, magnetic or electric force, etc.). In some cases, the loading of the droplet and the bead is super-Poissonian. In other examples of pairs of microwells, the wells are configured to hold two droplets comprising different reagents and/or samples, which are merged upon contact or upon application of a stimulus. In such instances, the droplet of one microwell of the pair can comprise reagents that may react with an agent in the droplet of the other microwell of the pair. For instance, one droplet can comprise reagents that are configured to release the nucleic acid barcode molecules of a bead contained in another droplet, located in the adjacent microwell. Upon merging of the droplets, the nucleic acid barcode molecules may be released from the bead into the partition (e.g., the microwell or microwell pair that are in contact), and further processing may be performed (e.g., barcoding, nucleic acid reactions, etc.). In cases where intact or live cells are loaded in the microwells, one of the droplets may comprise lysis reagents for lysing the cell upon droplet merging.

A droplet or bead may be partitioned into a well. The droplets may be selected or subjected to pre-processing prior to loading into a well. For instance, the droplets may comprise cells, and only certain droplets, such as those containing a single cell (or at least one cell), may be selected for use in loading of the wells. Such a pre-selection process may be useful in efficient loading of single cells, such as to obtain a non-Poissonian distribution, or to pre-filter cells for a selected characteristic prior to further partitioning in the wells. Additionally, the technique may be useful in obtaining or preventing cell doublet or multiplet formation prior to or during loading of the microwell.

In some instances, the wells can comprise nucleic acid barcode molecules attached thereto. The nucleic acid barcode molecules may be attached to a surface of the well (e.g., a wall of the well). The nucleic acid barcode molecules may be attached to a droplet or bead that has been partitioned into the well. The nucleic acid barcode molecule (e.g., a partition barcode sequence) of one well may differ from the nucleic acid barcode molecule of another well, which can permit identification of the contents contained with a single partition or well. In some cases, the nucleic acid barcode molecule can comprise a spatial barcode sequence that can identify a spatial coordinate of a well, such as within the well array or well plate. In some cases, the nucleic acid barcode molecule can comprise a unique molecular identifier for individual molecule identification. In some instances, the nucleic acid barcode molecules may be configured to attach to or capture a nucleic acid molecule within a sample or cell distributed in the well. For example, the nucleic acid barcode molecules may comprise a capture sequence that may be used to capture or hybridize to a nucleic acid molecule (e.g., RNA, DNA) within the sample. In some instances, the nucleic acid barcode molecules may be releasable from the microwell. In some instances, the nucleic acid barcode molecules may be releasable from the bead or droplet. For instance, the nucleic acid barcode molecules may comprise a chemical cross-linker which may be cleaved upon application of a stimulus (e.g., photo-, magnetic, chemical, biological, stimulus). The nucleic acid barcode molecules, which may be hybridized or configured to hybridize to a sample nucleic acid molecule, may be collected and pooled for further processing, which can include nucleic acid processing (e.g., amplification, extension, reverse transcription, etc.) and/or characterization (e.g., sequencing). In some instances nucleic acid barcode molecules attached to a bead in a well may be hybridized to sample nucleic acid molecules, and the bead with the sample nucleic acid molecules hybridized thereto may be collected and pooled for further processing, which can include nucleic acid processing (e.g., amplification, extension, reverse transcription, etc.) and/or characterization (e.g., sequencing). In such cases, the unique partition barcode sequences may be used to identify the cell or partition from which a nucleic acid molecule originated.

Characterization of samples within a well may be performed. Such characterization can include, in non-limiting examples, imaging of the sample (e.g., cell, cell bead, or cellular components) or derivatives thereof. Characterization techniques such as microscopy or imaging may be useful in measuring sample profiles in fixed spatial locations. For instance, when cells are partitioned, optionally with beads, imaging of each microwell and the contents contained therein may provide useful information on cell doublet formation (e.g., frequency, spatial locations, etc.), cell-bead pair efficiency, cell viability, cell size, cell morphology, expression level of a biomarker (e.g., a surface marker, a fluorescently labeled molecule therein, etc.), cell or bead loading rate, number of cell-bead pairs, etc. In some instances, imaging may be used to characterize live cells in the wells, including, but not limited to dynamic live-cell tracking, cell-cell interactions (when two or more cells are co-partitioned), cell proliferation, etc. Alternatively, or in addition to, imaging may be used to characterize a quantity of amplification products in the well.

In operation, a well may be loaded with a sample and reagents, simultaneously or sequentially. When cells or cell beads are loaded, the well may be subjected to washing, e.g., to remove excess cells from the well, microwell array, or plate. Similarly, washing may be performed to remove excess beads or other reagents from the well, microwell array, or plate. In the instances where live cells are used, the cells may be lysed in the individual partitions to release the intracellular components or cellular analytes. Alternatively, the cells may be fixed or permeabilized in the individual partitions. The intracellular components or cellular analytes may couple to a support, e.g., on a surface of the microwell, on a solid support (e.g., bead), or they may be collected for further downstream processing. For instance, after cell lysis, the intracellular components or cellular analytes may be transferred to individual droplets or other partitions for barcoding. Alternatively, or in addition to, the intracellular components or cellular analytes (e.g., nucleic acid molecules) may couple to a bead comprising a nucleic acid barcode molecule; subsequently, the bead may be collected and further processed, e.g., subjected to nucleic acid reaction such as reverse transcription, amplification, or extension, and the nucleic acid molecules thereon may be further characterized, e.g., via sequencing. Alternatively, or in addition to, the intracellular components or cellular analytes may be barcoded in the well (e.g., using a bead comprising nucleic acid barcode molecules that are releasable or on a surface of the microwell comprising nucleic acid barcode molecules). The barcoded nucleic acid molecules or analytes may be further processed in the well, or the barcoded nucleic acid molecules or analytes may be collected from the individual partitions and subjected to further processing outside the partition. Further processing can include nucleic acid processing (e.g., performing an amplification, extension) or characterization (e.g., fluorescence monitoring of amplified molecules, sequencing). At any convenient or useful step, the well (or microwell array or plate) may be sealed (e.g., using an oil, membrane, wax, etc.), which enables storage of the assay or selective introduction of additional reagents.

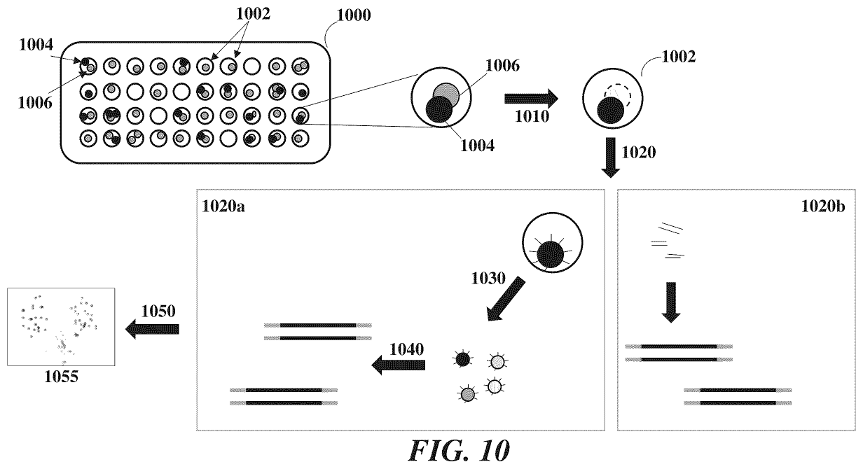

FIG. 10 schematically shows an example workflow for processing nucleic acid molecules within a sample (e.g., dissociated cells or nuclei as described herein). A substrate 1000 comprising a plurality of microwells 1002 may be provided. A sample 1006 which may comprise a cell, cell bead, cellular components or analytes (e.g., proteins and/or nucleic acid molecules) can be co-partitioned, in a plurality of microwells 1002, with a plurality of beads 1004 comprising nucleic acid barcode molecules. During process 1010, the sample 1006 may be processed within the partition. For instance, in the case of live cells, the cell may be subjected to conditions sufficient to lyse the cells and release the analytes contained therein. In process 1020, the bead 1004 may be further processed. By way of example, processes 1020a and 1020b schematically illustrate different workflows, depending on the properties of the bead 1004.

In 1020a, the bead comprises nucleic acid barcode molecules that are attached thereto, and sample nucleic acid molecules (e.g., RNA, DNA) may attach, e.g., via hybridization of ligation, to the nucleic acid barcode molecules. Such attachment may occur on the bead. In process 1030, the beads 1004 from multiple wells 1002 may be collected and pooled. Further processing may be performed in process 1040. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 1050, further characterization, such as sequencing may be performed to generate sequencing reads. The sequencing reads may yield information on individual cells or populations of cells, which may be represented visually or graphically, e.g., in a plot 1055.

In 1020b, the bead comprises nucleic acid barcode molecules that are releasably attached thereto, as described below. The bead may degrade or otherwise release the nucleic acid barcode molecules into the well 1002; the nucleic acid barcode molecules may then be used to barcode nucleic acid molecules within the well 1002. Further processing may be performed either inside the partition or outside the partition. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 1050, further characterization, such as sequencing may be performed to generate sequencing reads. The sequencing reads may yield information on individual cells or populations of cells, which may be represented visually or graphically, e.g., in a plot 755.

### IV. Sample Processing

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). In some instances, the biological sample comprises nucleic acids (such as DNA or RNA), proteins/polypeptides, carbohydrates, and/or lipids. In some embodiments, the biological sample is obtained as a tissue sample, such as a tissue section, biopsy, a core biopsy, needle aspirate, or fine needle aspirate. In some embodiments, the biological sample is or comprise a cell pellet or a section of a cell pellet. In some embodiments, the biological sample is or comprise a cell block or a section of a cell block. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Biological samples can be derived from a homogeneous culture or population of the subjects or organisms mentioned herein or alternatively from a collection of several different organisms. Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

In some embodiments, a substrate herein can be any support that is insoluble in aqueous liquid and which allows for positioning of biological samples, analytes, features, and/or reagents (e.g., probes) on the support. In some embodiments, a biological sample is attached to a substrate. Attachment of the biological sample can be irreversible or reversible, depending upon the nature of the sample and subsequent steps in the analytical method. In certain embodiments, the sample is attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose. In some embodiments, the substrate is coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Preparation

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick. More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, at least 0.2, at least 0.3, at least 0.4, at least 0.5, at least 0.7, at least 1.0, at least 1.5, at least 2, at least 3, at least 4, at least 5, at least 6, at least 7, at least 8, at least 9, at least 10, at least 12, at least 13, at least 14, at least 15, at least 20, at least 30, at least 40, or at least 50 μm. Thicker sections can also be used if desired or convenient, e.g., at least 70, 80, 90, or 100 μm or more. Typically, the thickness of a tissue section is between any of 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analyzed. In some instances, the thickness of a tissue section is between any of 1-100 μm, 1-50 μm, or 1-10 μm.

In some instances, multiple sections are obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analyzed successively to obtain three-dimensional information about the biological sample.

In some embodiments, the biological sample (e.g., a tissue section as described above) is prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

In some embodiments, the biological sample is prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes). In some embodiments, the biological sample (e.g., FFPE sample) is permeable after deparaffinization. In some embodiments, processing of the biological sample, such as de-waxing, allows the biological sample to become permeabilized.

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular or padlock probe. In some embodiments, one or more post-fixing step is performed after a hybridization complex comprising a probe and a target is formed in a sample. In some embodiments, one or more post-fixing step is performed prior to a ligation reaction disclosed herein.

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

In some embodiments, a biological sample is permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample is incubated with a cellular permeabilizing agent to facilitate permeabilization of the sample. Additional methods for sample permeabilization are described, for example, in Jamur et al., Method Mol. Biol. 588:63-66, 2010, which is herein incorporated by reference in its entirety. Any suitable method for sample permeabilization can generally be used in connection with the samples described herein.

In some embodiments, the biological sample can be permeabilized by any suitable methods. In some embodiments, the biological sample is a permeable biological sample. For example, one or more lysis reagents can be added to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes. Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

Additional reagents can be added to a biological sample to perform various functions prior to analysis of the sample. In some embodiments, DNase and RNase inactivating agents or inhibitors such as proteinase K, and/or chelating agents such as EDTA, is added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### (ii) Embedding

In some embodiments, the biological sample is embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample. Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some aspects, a biological sample can be embedded in any of a variety of other embedding materials to provide structural substrate to the sample prior to sectioning and other handling steps. In some cases, the embedding material is removed e.g., prior to analysis of tissue sections obtained from the sample. Suitable embedding materials include, but are not limited to, waxes, resins (e.g., methacrylate resins), epoxies, and agar.

In some embodiments, the biological sample is embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible or irreversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Pat. Pubs. 2017/0253918, 2018/0052081 and 2010/0055733, each of which is herein incorporated by reference in its entirety.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 m to about 2 mm.

Additional methods and aspects of hydrogel embedding of biological samples are described for example in Chen et al., Science 347(6221):543-548, 2015, which is herein incorporated by reference in its entirety.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible. In some embodiments, HTC reagents are added to the hydrogel before, contemporaneously with, and/or after polymerization. In some embodiments, a cell labelling agent is added to the hydrogel before, contemporaneously with, and/or after polymerization. In some embodiments, a cell-penetrating agent is added to the hydrogel before, contemporaneously with, and/or after polymerization.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and oligonucleotides. In some embodiments, optical labels are added to the hydrogel subunits before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a biological sample embedded in a matrix (e.g., a hydrogel) is isometrically expanded. Isometric expansion methods that can be used include hydration, a preparative step in expansion microscopy, as described in, e.g., Chen et al., Science 347(6221):543-548, 2015 and U.S. Pat. No. 10,059,990, which are herein incorporated by reference in their entireties. Isometric expansion of the sample can increase the spatial resolution of the subsequent analysis of the sample. The increased resolution in spatial profiling can be determined by comparison of an isometrically expanded sample with a sample that has not been isometrically expanded. In some embodiments, a biological sample is isometrically expanded to a size at least 2×, at least 2.1×, at least 2.2×, at least 2.3×, at least 2.4×, at least 2.5×, at least 2.6×, at least 2.7×, at least 2.8×, at least 2.9×, at least 3×, at least 3.1×, at least 3.2×, at least 3.3×, at least 3.4×, at least 3.5×, at least 3.6×, at least 3.7×, at least 3.8×, at least 3.9×, at least 4×, at least 4.1×, at least 4.2×, at least 4.3×, at least 4.4×, at least 4.5×, at least 4.6×, at least 4.7×, at least 4.8×, or at least 4.9× its non-expanded size. In some embodiments, the sample is isometrically expanded to at least 2× and less than 20× of its non-expanded size.

(iii) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. In some embodiments, one or more staining steps are performed to prepare or process a biological sample for an assay described herein or may be performed during and/or after an assay. In some embodiments, the sample is contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample is segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples is destained. Any suitable methods of destaining or discoloring a biological sample may be utilized and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, each of which is herein incorporated by reference in its entirety.

### V. Compositions and Systems

In some aspects, provided herein are compositions comprising any of the probes for generating a composite barcode as described in Section I and optionally reagents for amplification, and reagents for detection of generated amplification products (e.g., RCA products) described herein. Also provided herein are systems, for analyzing an analyte in a biological sample according to any of the methods described herein. In some embodiments, provided herein is a system comprising a plurality of oligonucleotide probes for detecting the multiple barcode sequences of the composite barcode or complements thereof in the cell at a location in the biological sample. Provided herein are reagents for performing single cell barcoding reaction. In some instances, the system comprises instructions for use, a microwell array or device and reagents (e.g., beads). In some embodiments, the system comprises any useful reagents for performing the processes described herein, e.g., nucleic acid reactions, barcoding of nucleic acid molecules, sample processing (e.g., for cell lysis, fixation, and/or permeabilization).

The various components of the system may be present in separate containers or certain compatible components may be pre-combined into a single container. In some embodiments, the system further contain instructions for using the components to practice the provided methods.

In some embodiments, the system comprises reagents and/or consumables required for performing one or more steps of the provided methods. In some embodiments, the system comprises reagents for fixing, embedding, and/or permeabilizing the biological sample. In some embodiments, the system comprises reagents for dissociating cells of the biological sample. In some embodiments, the system comprises reagents, such as enzymes and buffers for ligation and/or amplification, such as ligases and/or polymerases. In some aspects, the system comprises any of the reagents described herein, e.g., wash buffer and ligation buffer. In some embodiments, the system comprises reagents for detection by hybridization of probes and sequencing, such as detectably labeled probes and labeled nucleotides. In some embodiments, a system disclosed herein comprises a pool of detectably labeled probes each comprising a detectable label. In some embodiments, the system comprises optionally other components, for example nucleic acid primers for sequencing.

Provided herein are systems, for example comprising one or more oligonucleotides, e.g., any described in Sections I-IV, and instructions for performing the methods provided herein. In some embodiments, the system comprises one or more reagents for performing the methods provided herein. In some embodiments, the system comprises one or more reagents required for one or more steps comprising hybridization, ligation, extension, amplification, detection, and/or sample preparation, e.g., as described in Sections II-IV. In some embodiments, the system comprises any one or more of the reagents for nucleic acid analysis, e.g., as described in Section III. In some embodiments, any or all of the oligonucleotides are DNA molecules.

In some embodiments, the system comprises an enzyme such as a ligase and/or a polymerase described herein. In some embodiments, the system comprises a polymerase, for instance for performing extension of the for sequencing. In some embodiments, the system comprises a cell or tissue sample on a substrate (e.g., a slide). In some embodiments, the system comprises reagents for forming a functionalized matrix (e.g., a hydrogel), such as any suitable functional moieties. In some examples, also provided are buffers and reagents for tethering RCA products to a matrix (e.g., a hydrogel).

Provided herein is a system comprising a plurality of probes, wherein each probe of the plurality of probes each comprises a same target binding sequence and different barcode sequences; reagents for performing a single cell barcoding reaction; and a dissociation buffer. In some instances, the reagents for performing the single cell barcoding reaction comprises a plurality of first probe molecules for binding a panel of target analytes. In some aspects, the dissociation buffer comprises a protease and/or a collagenase. In some embodiments, the plurality of probes comprise a plurality of circularizable probes, and the system comprises one or more reagents for circularizing the plurality of circularizable probes. In some embodiments, one or more reagents for generating a plurality of rolling circle amplification products (RCPs) using the circularizable probes are included in the system. In some cases, the system comprises a plurality of a sequencing primer, a plurality of detectably labeled nucleotides, and a polymerase.

In some embodiments, the systems includes reagents and/or consumables required for performing one or more steps of the provided methods. In some embodiments, the systems contain reagents for fixing, embedding, and/or permeabilizing the biological sample. In some embodiments, the systems contain reagents, such as enzymes and buffers for ligation and/or amplification, such as ligases and/or polymerases. In some aspects, the system can also comprise any of the reagents described herein, e.g., wash buffer and ligation buffer. In some embodiments, the systems comprise reagents for detection and/or sequencing, such as detectably labeled oligonucleotides or detectable labels. In some embodiments, the systems optionally contain other components, for example nucleic acid primers, enzymes and reagents, buffers, nucleotides, modified nucleotides, reagents for additional assays.

The various components of the system may be present in separate containers or certain compatible components may be pre-combined into a single container. In some embodiments, the system further contain instructions for using the components to practice the provided methods.

### VI. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

A “primer” as used herein, in some embodiments, is an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

In some instances, “ligation” refers to the formation of a covalent bond or linkage between the termini of two or more nucleic acids, e.g., oligonucleotides and/or polynucleotides, in a template-driven reaction. The nature of the bond or linkage may vary widely and the ligation, in some embodiments, is carried out enzymatically or chemically. As used herein, ligations are usually carried out enzymatically to form a phosphodiester linkage between a 5′ carbon terminal nucleotide of one oligonucleotide with a 3′ carbon of another nucleotide.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

The terms “adaptor(s)”, “adapter(s)” and “tag(s)” may be used synonymously. An adaptor or tag can be coupled to a polynucleotide sequence to be “tagged” by any approach, including ligation, hybridization, or other approaches.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively, or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

As used herein, the term “barcoded nucleic acid molecule” generally refers to a nucleic acid molecule that results from, for example, the processing of a nucleic acid barcode molecule with a nucleic acid sequence (e.g., nucleic acid sequence complementary to a nucleic acid primer sequence encompassed by the nucleic acid barcode molecule). The nucleic acid sequence may be a targeted sequence or a non-targeted sequence. The nucleic acid barcode molecule may be coupled to or attached to the nucleic acid molecule comprising the nucleic acid sequence. For example, a nucleic acid barcode molecule described herein may be hybridized to an analyte (e.g., a messenger RNA (mRNA) molecule) of a cell. Reverse transcription can generate a barcoded nucleic acid molecule that has a sequence corresponding to the nucleic acid sequence of the mRNA and the barcode sequence (or a reverse complement thereof). The processing of the nucleic acid molecule comprising the nucleic acid sequence, the nucleic acid barcode molecule, or both, can include a nucleic acid reaction, such as, in non-limiting examples, reverse transcription, nucleic acid extension, ligation, etc. The nucleic acid reaction may be performed prior to, during, or following barcoding of the nucleic acid sequence to generate the barcoded nucleic acid molecule. For example, the nucleic acid molecule comprising the nucleic acid sequence may be subjected to reverse transcription and then be attached to the nucleic acid barcode molecule to generate the barcoded nucleic acid molecule, or the nucleic acid molecule comprising the nucleic acid sequence may be attached to the nucleic acid barcode molecule and subjected to a nucleic acid reaction (e.g., extension, ligation) to generate the barcoded nucleic acid molecule. A barcoded nucleic acid molecule may serve as a template, such as a template polynucleotide, that can be further processed (e.g., amplified) and sequenced to obtain the target nucleic acid sequence. For example, in the methods and systems described herein, a barcoded nucleic acid molecule may be further processed (e.g., amplified) and sequenced to obtain the nucleic acid sequence of the nucleic acid molecule (e.g., mRNA).

The term “biological particle” may be used herein to generally refer to a discrete biological system derived from a biological sample. The biological particle may be a macromolecule. The biological particle may be a small molecule. The biological particle may be a virus. The biological particle may be a cell or derivative of a cell. The biological particle may be an organelle. The biological particle may be a nucleus of a cell. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be a constituent of a cell. The biological particle may be or may include DNA, RNA, organelles, proteins, or any combination thereof. The biological particle may be or may include a matrix (e.g., a gel or polymer matrix) comprising a cell or one or more constituents from a cell (e.g., cell bead), such as DNA, RNA, organelles, proteins, or any combination thereof, from the cell. The biological particle may be obtained from a tissue of a subject. The biological particle may be a hardened cell. Such hardened cell may or may not include a cell wall or cell membrane. The biological particle may include one or more constituents of a cell but may not include other constituents of the cell. An example of such constituents is a nucleus or an organelle. A cell may be a live cell. The live cell may be capable of being cultured, for example, being cultured when enclosed in a gel or polymer matrix or cultured when comprising a gel or polymer matrix.

The term “macromolecular constituent,” as used herein, generally refers to a macromolecule contained within or from a biological particle. The macromolecular constituent may comprise a nucleic acid. In some cases, the biological particle may be a macromolecule. The macromolecular constituent may comprise DNA. The macromolecular constituent may comprise RNA. The RNA may be coding or non-coding. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

The term “partition,” as used herein, generally, refers to a space or volume that may be suitable to contain one or more species or conduct one or more reactions. A partition can be a physical container, compartment, or vessel, such as a droplet, a flowcell, a reaction chamber, a reaction compartment, a tube, a well, or a microwell. The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

### EXAMPLES

The following examples are included for illustrative purposes only and are not intended to limit the scope of the present disclosure.

### Example 1: Cell Dissociation and Recovery after In Situ Detection of Barcodes

This example demonstrates a workflow for recovering dissociated cells after performing in situ detection of amplification products generated using hybridized circularizable probes.

In this example, paraffin embedded formalin fixed (FFPE) mouse brain tissue samples were cryosectioned onto glass slides for processing and deparaffinized. A plurality of circularizable probes were contacted with the sample and ligated to form circularized probes upon hybridization to a panel of target nucleic acid sequences in the biological samples. Each circularizable probe had a target hybridization region designed such that hybridization to the target nucleic acid sequence positioned the ligatable 5′ end and 3′ end of the circularizable probe into proximity for ligation. Each circularizable probe also had a barcode region. The probes were incubated with the tissue sections and hybridization buffer for hybridization of the probe to the target nucleic acid sequences in the sample. After probe hybridization, the probe hybridization mixture was removed and the samples were washed with PBST to remove unbound probes.

The circularizable probes were then ligated to generate circularized probes, and the tissue sections were then washed in PBST. For generating rolling circle amplification (RCA) products, the samples were incubated at 30° C. in a reaction mix containing a Phi29 polymerase buffer, dNTPs, and Phi29 polymerase. The generated rolling circle amplification products (RCPs) were detected by performing sequential cycles of hybridizing labelled probes to the RCPs in situ and imaging the samples with a fluorescent microscope.

For dissociation, 10 μM tissue samples were individually minced, placed in tubes, and spun to obtain tissue pellets. The resuspended tissue pellet was treated with a working solution with Liberase (0.2 mg/mL) in RPMI media and loaded onto a heated dissociator with the indicated programs for cell resuspension indicated in Table 1 with an agitation step at various times, indicated by (S). The mixtures were resuspended with the same media to obtain a total volume of 500 μL, then filtered, spun down, and 400 uL was retained for cell counting. A portion of two of the samples were stained with DAPI by incubating with the stain for 10 min. A 100 μL portion of the sample was counted and the resulting concertation of cells recovered are shown in Table 1. The cell dissociation can be improved by varying conditions such as by incubating the sample at 37° C., manually shaking the tube intermittently, using a silanized glass pipette to triturate the tissue pieces at least 15-20× (for example, until the solution begins to turn cloudy), and/or further removing debris from the sample by filtering (for example, using a 70 μm filter to remove debris and undissociated tissue pieces).

TABLE 1
Cell Dissociation and Recovery

| | Tissue type | | Dissociation | | DAPI | Concentration |
|---|---|---|---|---|---|---|
| Sample | (thickness) | Program for cell resuspension | Temp | Filter | stained | x10e5/mL |
| 1 | Mouse brain | 20 min 300 RPM + (S) + 30 min | 37° C. | 30 μm | yes | 3.90 |
| | (10 μm) | 500 rpm + 30 min 500 rpm | | | | |
| 2 | Mouse brain | 20 min 300 RPM + 30 min 500 | 37° C. | 30 μm | yes | 2.05 |
| | (10 μm) | rpm + (S) + 30 min 500 rpm | | | | |
| 3 | Mouse brain | 20 min 300 RPM + (S) + 30 min | 37° C. | 30 μm | no | 4.18 |
| | (10 μm) | 500 rpm + 30 min 500 rpm | | | | |
| 4 | Mouse brain | 20 min 300 RPM + 30 min 500 | 37° C. | 30 μm | no | 3.07 |
| | (10 μm) | rpm + (S) + 30 min 500 rpm | | | | |

The results show that the cells from a biological sample tissue section were removed from the substrate, and sufficiently recovered for downstream analysis. For example, the methods provided herein include further performing a single cell barcoding reaction to append a cell barcode to sequences of or associated with analytes and nucleic acid molecules (e.g., generated RCPs detected as described in this Example) and perform downstream sequencing to correlate location information with the barcoded analyte sequences in the biological sample. To achieve sufficient introduction of barcodes to generate a composite barcode such that a plurality of different barcodes are introduced into each single cell or nucleus, a plurality of probes that target an abundant transcript such as actin beta (ACTB) can be contacted with the sample and ligated to form circularized probes upon hybridization, amplified, and detected as described in this Example.

The present disclosure is not intended to be limited in scope to the particular disclosed embodiments, which are provided, for example, to illustrate various aspects of the disclosure. Various modifications to the compositions and methods described will become apparent from the description and teachings herein. Such variations may be practiced without departing from the true scope and spirit of the disclosure and are intended to fall within the scope of the present disclosure.

1. A method of analyzing a biological sample, comprising:
(a) contacting a plurality of cells in the biological sample with a plurality of probes to generate a composite barcode in a cell of the plurality of cells,
wherein the probes of the plurality of probes comprise a target binding sequence and a barcode sequence, wherein the target binding sequences of the probes in the plurality of probes are the same, and the probes of the plurality of probes comprise different barcode sequences;
wherein the composite barcode in the cell comprises two or more different barcode sequences from separate probes of the plurality of probes;
(b) detecting the two or more different barcode sequences of the composite barcode or complements thereof in the cell at a location in the biological sample;
(c) dissociating cells or nuclei of the plurality of cells from the biological sample;
(d) subjecting the dissociated cells or dissociated nuclei of the cells to a single cell barcoding reaction to append a common cell barcode or complement thereof to (i) a nucleic acid molecule comprising a barcode sequence of the two or more different barcode sequences of the composite barcode, and (ii) a sequence of or associated with an analyte from the cell, thereby generating a plurality of barcoded nucleic acid products; and
(e) using the common cell barcode and the location of the detected composite barcode to locate the analyte in the biological sample.

2. The method of claim 1, wherein (d) comprises partitioning the dissociated cells or dissociated nuclei with a plurality of nucleic acid barcode molecules in a partition, wherein nucleic acid barcode molecules of the plurality of nucleic acid barcode molecules comprises a capture sequence and the cell barcode or complement thereof.

3-6. (canceled)

7. The method of claim 2, further comprising sequencing the plurality of barcoded nucleic acid products or derivatives thereof.

8-13. (canceled)

14. The method of claim 1, wherein in (d), the cell barcode or complement thereof is appended to a plurality of analytes each bound by a labelling agent.

15-19. (canceled)

20. The method of claim 1, wherein a first probe molecule is directly or indirectly bound to the analyte prior to (d) or in (d).

21-24. (canceled)

25. The method of claim 20, wherein a second probe molecule is directly or indirectly bound to the analyte prior to (d) or in (d).

26-31. (canceled)

32. The method of claim 1, wherein the plurality of probes comprise a plurality of circularizable probes and the method comprises ligating the circularizable probes to generate a plurality of circularized probes and performing rolling circle amplification (RCA) using the circularized probes as template to generate a plurality of RCA products in the plurality of cells.

33-40. (canceled)

41. The method of claim 1, wherein the target binding sequence hybridizes to a transcript of a housekeeping gene.

42. The method of claim 1, wherein the target binding sequence hybridizes to a transcript of a commonly expressed gene expressed by at least 2 different cell types.

43. (canceled)

44. The method of claim 42, wherein the commonly expressed gene is expressed in at least 50% of cells in the biological sample.

45-46. (canceled)

47. The method of claim 42, wherein the commonly expressed gene has a mean count of more than 20 transcripts per cell by single cell RNA sequencing.

48-52. (canceled)

53. The method of claim 1, wherein (a) comprises contacting the plurality of cells with an additional plurality of probes, wherein the additional plurality of probes each comprises an additional target binding sequence and an additional barcode sequence, and wherein the additional target binding sequence in the additional plurality of probes are the same, and the additional barcode sequences in the additional plurality of probes are different.

54-55. (canceled)

56. The method of claim 1, wherein the composite barcode comprises at least 3 different barcode sequences.

57. The method of claim 1, wherein the plurality of probes comprises at least 5 or more separate probes comprising the same target binding sequence.

58. The method of claim 1, wherein the plurality of probes comprises at least 10 or more different barcode sequences.

59-60. (canceled)

61. The method of claim 1, wherein detecting the two or more different barcode sequences or complements thereof comprises binding a plurality of intermediate probes directly or indirectly to the barcode sequences or complements thereof, binding a detectably labeled probes directly or indirectly to detection regions of the intermediate probes, and detecting signals associated with the detectably labeled probes.

62. (canceled)

63. The method of claim 1, wherein detecting the two or more different barcode sequences or complements thereof comprises sequencing all or a portion of the two or more different barcode sequences or complements thereof using sequencing-by-ligation (SBL) or sequencing-by-synthesis (SBS).

64-66. (canceled)

67. The method of claim 1, wherein each of the two or more different barcode sequences of the composite barcode is assigned a sequence of signal codes that identifies the barcode sequence and wherein detecting the two or more different barcode sequences or complements thereof comprises detecting the corresponding sequences of signal codes detected from sequential hybridization, detection, and removal of sequential pools of intermediate probes and the universal pool of detectably labeled probes.

68-71. (canceled)

72. The method of claim 1, wherein (b) comprises associating the composite barcode with the location of the cell in the biological sample.

73. The method of claim 1, wherein the cell barcode or complement thereof associates the composite barcode with the sequence of or associated with the analyte from the same single cell or nucleus.

74-99. (canceled)
